(19) United States
(12) Patent Application Publication
(10) Pub. No.: US 2023/0193232 A1
(43) Pub. Date: Jun. 22, 2023

(54) RECOMBINANT YEAST HOST CELL EXPRESSING AN HYDROLASE

(71) Applicant: Lallemand Hungary Liquidity Management LLC, Budapest (HU)

(72) Inventors: Trisha Barrett, Bradford, VT (US); Aaron Argyros, Lebanon, NH (US)

(21) Appl. No.: 18/049,055

(22) Filed: Oct. 24, 2022

Related U.S. Application Data
(63) Continuation of application No. 17/317,443, filed on May 11, 2021, now abandoned.
(60) Provisional application No. 63/022,960, filed on May 11, 2020.

(51) Int. Cl. C12N 9/24; C12N 1/18; C12N 9/10; C12N 9/48; C12P 7/10

(52) U.S. Cl. CPC C12N 9/2402; C12N 1/18; C12N 9/48; C12N 9/1051; C12N 9/2405; C12P 7/10; C12Y 204/01001; C12Y 302/01028

(57) ABSTRACT

The present disclosure concerns a recombinant yeast host cell exhibiting higher stability and, in some embodiments, higher fermentation performance. The recombinant yeast host cell stability has a limited ability to express an hydrolase during its propagation phase. In return, this limits the cleavage of a yeast cellular component during or after propagation which may be detrimental to the stability and/or fermentation performances. The recombinant yeast host cell expresses a heterologous hydrolase under the control of a heterologous promoter (for limiting the expression of the heterologous hydrolase during propagation and favoring the expression of the heterologous hydrolase during fermentation).

### REFERENCE TO AN ELECTRONIC SEQUENCE LISTING

The contents of the electronic sequence listing (580127_431C1_SEQUENCE_LISTING.xml; Size: 92,238 bytes; and Date of Creation: Oct. 21, 2022) is herein incorporated by reference in its entirety.

### TECHNOLOGICAL FIELD

The present disclosure concerns a recombinant yeast host cell modified to express a heterologous hydrolase (capable of hydrolyzing a yeast cellular component) under the control of a promoter limiting the expression of the heterologous hydrolase during propagation and favoring the expression of the heterologous hydrolase during propagation.

### BACKGROUND

In consolidated bioprocessing applications, Saccharomyces cerevisiae is genetically engineered to express heterologous polypeptides for improving fermentation yield or improving robustness. Prior to fermentation, the yeast must be propagated. The propagated yeasts are then transferred to a fermentation medium (which usually differs from the propagation medium). The propagated yeasts can be stored, for a few hours or a few days, prior to the fermentation.

It would be highly desirable to be provided with a recombinant host cell exhibiting an improved stability/viability prior to fermentation, especially during propagation and/or storage and/or improved fermentation performances.

### BRIEF SUMMARY

The present disclosure concerns a recombinant yeast host cell exhibiting increased stability/maintained viability during storage and/or an improved fermentation performance. The recombinant yeast host cell of the present disclosure has an “improved fermentation performance” when compared to another yeast host cell expressing the same hydrolase, but under the control of another promoter (constitutive or favoring the expression of the heterologous hydrolase during propagation). The improved in fermentation performance can be observed, for example, in the amount of CO₂ produced, the fermentation product yield (e.g., alcohol such as, for example, ethanol), the sugar consumption (e.g., DP1 such as, for example glucose and/or DP2 such as for example trehalose), the amount of glycerol produced, etc. The recombinant yeast host cell of the present disclosure has an “increase stability” or a “maintained viability” when compared to another yeast host cell expressing the same hydrolase, but under the control of another promoter (constitutive or favoring the expression of the heterologous hydrolase during propagation). The increased stability/maintained viability can be observed, for example, in the weight of the cells, the viability of the cells, the intracellular trehalose content and/or the reduced ability of the recombinant yeast host cell in to convert a unfermentable carbohydrate source into a fermentable carbohydrate source (when compared to another yeast host cell expressing the same hydrolase, but under the control of another promoter (constitutive or favoring the expression of the heterologous hydrolase during propagation)).

According to a first aspect, the present disclosure provides a recombinant yeast host cell capable of expressing a first heterologous polypeptide under the control of a heterologous promoter. The first heterologous polypeptide is an hydrolase. The heterologous promoter is capable of limiting the expression of the first heterologous polypeptide during a propagation and favoring the expression of the first heterologous polypeptide during a fermentation. The hydrolase is capable of generating an enzymatic product from a substrate. The substrate is a yeast cellular component which can be, for example, an intracellular component, a component associated to yeast cell membrane and/or a component associated to the yeast cell wall. In an embodiment, the hydrolase is a glycoside hydrolase. In another embodiment, the glycoside hydrolase is for converting an unfermentable carbohydrate source (e.g., the substrate) into a fermentable carbohydrate source (e.g., the enzymatic product). In an embodiment, trehalose is the substrate, the intracellular component and/or the unfermentable carbohydrate source. In some additional embodiments, the first heterologous enzyme is a trehalase. The trehalase can have, for example, (a) the amino acid sequence of any one of SEQ ID NO.: 7 or 14 to 21; be (b) a variant of the amino acid sequence of (a) exhibiting trehalase activity; or be (c) a fragment of the amino acid sequence of (a) or (b) exhibiting trehalase activity. In an embodiment, the trehalase is from Neurospora sp. and, in a further embodiment, from Neurospora crassa. In such embodiment, the trehalase can have the amino acid sequence of SEQ ID NO: 7, can be a variant of the amino acid sequence of SEQ ID NO: 7 exhibiting trehalase activity or can be a fragment of the amino acid sequence of SEQ ID NO: 7 or the variant and exhibiting trehalase activity. In another embodiment, the hydrolase is a peptide hydrolase. In such embodiment, a polypeptide or a peptide is the substrate. In such embodiment, the first heterologous polypeptide can be, for example, a protease. In some embodiments, the protease is from Candida sp. and in some specific embodiments, from Candida albicans. In some specific embodiments, the protease has the amino acid sequence of SEQ ID NO: 37, is a variant of the amino acid sequence of SEQ ID NO: 37 exhibiting protease activity or is a fragment of the amino acid sequence of SEQ ID NO: 37 exhibiting protease activity. In some additional embodiments of the hydrolase being a glycoside hydrolase, glycogen is the substrate. In such embodiment, the first heterologous polypeptide can comprise, for example, a glycogen phosphorylase and/or a glycogen debranching enzyme. In still another embodiment, the hydrolase is a glucan hydrolase. In such embodiment, glucan is the substrate. In another embodiment, β-glucan is the substrate. In such embodiments, the first heterologous polypeptide can be, for example, a glucanase. In an embodiment, the cleavage of the substrate and/or the accumulation of the enzymatic product, if generated prior to the fermentation, is detrimental to the performance of the recombinant yeast host cell during the fermentation. In an embodiment, the recombinant yeast host cell is capable of modifying the enzymatic product is capable into an inhibitory product detrimental to the performance of the recombinant yeast host cell during the fermentation, if generated prior to the fermentation. In an embodiment, the inhibitory product is an alcohol, such as, for example ethanol. In another embodiment, the propagation is an aerobic propagation (e.g., performed under aerobic conditions). In still another embodiment, the fermentation is an anaerobic fermentation (e.g., performed under anaerobic conditions). In a further embodiment, the recombinant yeast host cell is capable of expressing one or more second heterologous polypeptide, wherein the one or more second heterologous polypeptide is a saccharolytic enzyme and, in a further embodiment, the saccharolytic enzyme can be a glucoamylase. In some embodiments, the glucoamylase can have the amino acid sequence of SEQ ID NO: 1, 32 or 34, be a variant of the amino acid sequence of SEQ ID NO: 1, 32 or 34 exhibiting glucoamylase activity or be a fragment of the amino acid sequence of SEQ ID NO: 1, 32 or 34 exhibiting glucoamylase activity. In still another embodiment, the recombinant yeast host cell is capable of expressing one or more third heterologous polypeptide for modulating the production of formate. In one example, the one or more third heterologous polypeptide comprises PFLA. In such embodiment, PFLA can have the amino acid sequence of SEQ ID NO: 3, be a variant of amino acid sequence of SEQ ID NO: 3 exhibiting pyruvate formate lyase activity or be a fragment of the amino acid sequence of SEQ ID NO: 3 exhibiting pyruvate formate lyase activity. In another example, the one or more third heterologous polypeptide comprises PFLB. In such embodiment, PFLB can have the amino acid sequence of SEQ ID NO: 4, be a variant of amino acid sequence of SEQ ID NO: 4 exhibiting pyruvate formate lyase activity or be a fragment of the amino acid sequence of SEQ ID NO: 4 exhibiting pyruvate formate lyase activity. In still another example, the one or more third heterologous polypeptide comprises FDH1. In such embodiments, FDH1 can have the amino acid sequence of SEQ ID NO: 5, be a variant of the amino acid sequence of SEQ ID NO: 5 exhibiting formate dehydrogenase activity or be a fragment of the amino acid sequence of SEQ ID NO: 5 exhibiting formate dehydrogenase activity. In another embodiment, the recombinant yeast host cell is capable of expressing one or more fourth heterologous polypeptide for converting acetyl-CoA into an alcohol. In an example, the one or more fourth heterologous polypeptide comprises ADHE. In such embodiment, ADHE can have the amino acid sequence of SEQ ID NO: 2, be a variant of the amino acid sequence of SEQ ID NO: 2 exhibiting acetaldehyde/alcohol dehydrogenase activity or be a fragment of the amino acid sequence of SEQ ID NO: 2 exhibiting acetaldehyde/alcohol dehydrogenase activity. In yet another embodiment, the recombinant yeast host cell is capable of expressing one or more fifth heterologous polypeptide involved in the production of glycerol, in the regulation of the production of glycerol or in the transport of glycerol. In one example, the one or more fifth heterologous polypeptide comprises STL1. In such embodiment, STL1 can have the amino acid sequence of SEQ ID NO 6, be a variant of the amino acid sequence of SEQ ID NO: 6 exhibiting glycerol transport activity or be a fragment of the amino acid sequence of SEQ ID NO: 6 exhibiting glycerol transport activity. In still a further embodiment, the recombinant yeast host cell is capable of expressing one or more sixth heterologous polypeptide involved in producing trehalose and/or in regulating trehalose production. In one example, the one or more sixth heterologous polypeptide comprises TSL1. In such embodiment, TSL1 can have the amino acid sequence of SEQ ID NO: 13, be a variant of the amino acid sequence of SEQ ID NO: 13 exhibiting trehalose production regulatory activity or be a fragment of the amino acid sequence of SEQ ID NO: 13 exhibiting trehalose production regulatory activity. Furthermore, the one or more sixth heterologous polypeptide (such as TSL1) can be under the control of a modified tsl1 promoter, such as the promoter having the nucleotide sequence of SEQ ID NO: 35, a variant thereof or a fragment thereof. In still a further embodiment, the recombinant yeast host cell is capable of expressing one or more seventh heterologous polypeptide having glyceraldehyde-3-phosphate dehydrogenase activity. In one example, the one or more seventh heterologous polypeptide comprises GAPN. In such embodiment, GAPN can have the amino acid sequence of SEQ ID NO: 33, be a variant of SEQ ID NO: 33 exhibiting glyceraldehyde-3-phosphate dehydrogenase activity or be a fragment of SEQ ID NO: 33 glyceraldehyde-3-exhibiting phosphate dehydrogenase activity. In an embodiment, the heterologous promoter is fermentation specific promoter such as, for example, an anaerobic specific promoter. In some embodiments, the anaerobic specific promoter comprises a promoter from the tir1 gene, the pau5 gene, the dan1 gene, the tdh1 gene, the spi1 gene, the hxk1 gene, the anb1 gene, the hxt6 gene, the trx1 gene and/or the aac3 gene. In an embodiment, the promoter of the tir1 gene can have the nucleotide sequence of SEQ ID NO: 10, be variant of the nucleotide sequence of SEQ ID NO: 10 or be fragment of the nucleotide sequence of SEQ ID NO: 10. In another embodiment, the promoter of the pau5 gene can have the nucleotide sequence of SEQ ID NO: 11, be variant of the nucleotide sequence of SEQ ID NO: 11 or be fragment of the nucleotide sequence of SEQ ID NO: 11. In still another embodiment, the promoter of the dan1 gene can have the nucleotide sequence of SEQ ID NO: 12, be variant of the nucleotide sequence of SEQ ID NO: 12 or be fragment of the nucleotide sequence of SEQ ID NO: 12. In another embodiment the promoter of the tdh1 gene has the nucleotide sequence of SEQ ID NO: 39, is a variant of the nucleotide sequence of SEQ ID NO: 39 or is a fragment of the nucleotide sequence of SEQ ID NO: 39. In still another embodiment, the promoter of the spi1 gene has the nucleotide sequence of SEQ ID NO: 40, is a variant of the nucleotide sequence of SEQ ID NO: 40 or is a fragment of the nucleotide sequence of SEQ ID NO: 40. In yet another embodiment, the promoter of the hxk1 gene has the nucleotide sequence of SEQ ID NO: 41, is a variant of the nucleotide sequence of SEQ ID NO: 41 or is a fragment of the nucleotide sequence of SEQ ID NO: 41. In a further embodiment, the promoter of the anb1 gene has the nucleotide sequence of SEQ ID NO: 42, is a variant of the nucleotide sequence of SEQ ID NO: 42 or is a fragment of the nucleotide sequence of SEQ ID NO: 42. In still yet another embodiment, the promoter of the hxt6 gene has the nucleotide sequence of SEQ ID NO: 43, is a variant of the nucleotide sequence of SEQ ID NO: 43 or is a fragment of the nucleotide sequence of SEQ ID NO: 43. In yet another embodiment, the promoter of the trx1 gene has the nucleotide sequence of SEQ ID NO: 44, is a variant of the nucleotide sequence of SEQ ID NO: 44 or is a fragment of the nucleotide sequence of SEQ ID NO: 44. In still a further embodiment, the promoter of the aac3 gene has the nucleotide sequence of SEQ ID NO: 45, is a variant of the nucleotide sequence of SEQ ID NO: 45 or is a fragment of the nucleotide sequence of SEQ ID NO: 45. In an embodiment, the recombinant yeast host cell is from the genus Saccharomyces sp. and, in a further embodiment, from the species Saccharomyces cerevisiae.

According to a second aspect, the present disclosure provides a process for obtaining a population of propagated recombinant yeast host cells. The process comprises contacting the recombinant yeast host cell described herein with a propagation medium under conditions so as to allow the propagation of the recombinant yeast host cell to obtain the propagated recombinant yeast host cell population. In some embodiments, the process further comprises adding a stabilizer to the propagated recombinant yeast host cell population. In some embodiments, the stabilizer can be a polyol, like, for example, glycerol. In some embodiments, the substrate is a carbohydrate. In yet another embodiment, the carbohydrate is a source of unfermentable carbohydrate (such as, for example trehalose). In some further embodiments, the process further comprises storing the propagated recombinant yeast host cell population.

According to a third aspect, the present disclosure provides a population of propagated recombinant yeast host cells obtainable or obtained by the process described herein. In some embodiment, the population comprises a stabilizer. In some embodiments, the stabilizer is polyol, like, for example, glycerol.

According to a fourth aspect, the present disclosure provides a yeast composition comprising a population comprising the recombinant yeast host cell described herein and a stabilizer. In one embodiment, the stabilizer is a polyol, like, for example, glycerol In yet additional embodiments, the carbohydrate is a fermentable carbohydrate, such as, for example, glucose.

According to a fifth aspect, the present disclosure provides a process for converting a biomass into a fermentation product. The process comprises contacting the biomass with the recombinant yeast host cell described herein, the population described herein or the yeast composition described herein under conditions to allow the conversion of at least a part of the biomass into the fermentation product. In an embodiment, the biomass comprises corn, and in some specific embodiments, the corn can be provided as a mash. In a further embodiment, the fermentation product is ethanol.

### BRIEF DESCRIPTION OF THE DRAWINGS

Having thus generally described the nature of the invention, reference will now be made to the accompanying drawings, showing by way of illustration, a preferred embodiment thereof, and in which:

FIG. 1 illustrates the foaming and bubbling observed in the SLY obtained from strain M19481.

FIG. 2 illustrates the trehalase activity of different strains/transformants grown aerobically (white bars) or anaerobically (black bars). Results are shown as the absorbance at 540 nm in function of the different strains/transformants used. Results are shown for strains M2390 (wild-type) or M19481 (expressing a heterologous trehalase using the tef2 promoter) as well as for transformants of the strain M20398 expressing the same heterologous trehalase under the control of the tef2 promoter (TEF2p), the tir1 promoter (TIR1p), the pau5 promoter (PAU5p) or the dan1 promoter (DAN1p).

FIG. 3 illustrates the dry cell weight (DCW, in % w/w) in the SLY obtained from strains M19399 (long dashed line, which does not express a heterologous trehalase), M20790 (short dashed line, which expresses a heterologous trehalase under the control of an anaerobic promoter) and M19481 (full line, which expresses a heterologous trehalase under the control of an aerobic promoter) over time (days).

FIG. 4 illustrates the intracellular trehalose content (% w/w DCW) in the SLY obtained from strains M19399 (which does not express a heterologous trehalase), M19481 (which expresses a heterologous trehalase under the control of an aerobic promoter) and M20790 (which expresses a heterologous trehalase under the control of an anaerobic promoter). Results are shown as the weight percent of intracellular trehalose (in function of the total dry cell weight) at harvest (white bars), 1 day post-harvest (black bars) and 8 days post-harvest (diagonal hatch bars).

FIG. 5 illustrates the ethanol content (% w/v) in the supernatant of the SLY obtained from strains M19399 (long dashed line, which does not express a heterologous trehalase), M20790 (short dashed line, which expresses a heterologous trehalase under the control of an anaerobic promoter) and M19481 (full line, which expresses a heterologous trehalase under the control of an aerobic promoter) over time (days).

FIG. 6 illustrates the ethanol (g/L, white bars, left axis), glucose (g/L, ▲, right axis) and glycerol (g/L, • right axis) content of a corn fermentation using strains M2390, M15419 and M21211 under non-permissive conditions (high temperature or in the presence of lactic acid) or permissive conditions.

FIG. 7 illustrates the ethanol (g/L, white bars, left axis), glucose (g/L, •, right axis), glycerol (g/L, ■ right axis) and DP2 (g/L, ▲, right axis) content of a corn fermentation using strains M2390 and M23293 under permissive conditions.

FIG. 8 illustrates the ethanol (g/L, bars, left axis), glucose (g/L, •, right axis), glycerol (g/L, ▲, right axis) and glucose (g/L, ♦, right axis) content obtained after a corn fermentation using strains M2390, M10874 and M21757 under permissive conditions.

FIG. 8 provides the biological sequences of the present application.

FIG. 9 provides the absorbance (obtained at 540 nm) of the supernatant of various recombinant yeast host cell expressing a heterologous glucoamylase under the control of various promoters cultured in aerobic (grey bars) and anaerobic (black bars) conditions.

FIG. 10 provides the absorbance (obtained at 540 nm) of the supernatant of various recombinant yeast host cell expressing a heterologous xylanase under the control of various promoters cultured in aerobic (grey bars) and anaerobic (black bars) conditions.

### DETAILED DESCRIPTION

In accordance with the present disclosure, there is provided a recombinant yeast host cell exhibiting increased stability prior to fermentation and/or an improved fermentation performance. The recombinant yeast host cell expresses a first heterologous polypeptide (e.g., an hydrolase) under the control of a heterologous (e.g., fermentation specific) promoter (which limits the expression of the first heterologous polypeptide during propagation and/or favors the expression of the first heterologous polypeptide during fermentation). The heterologous hydrolase which can be expressed in the recombinant yeast host cell is capable or has the ability of converting a substrate into an enzymatic product. The substrate is a yeast cellular component. As used in the context of the present disclosure, a yeast cellular component refers to a biological component physically associated to the yeast and which can be metabolized by the yeast. The substrate or yeast cellular component can but does not need to be metabolically produced by the recombinant yeast host cell. For example, the substrate or yeast cellular component can be a stabilizer which is placed in contact with the recombinant yeast host cell and has the ability to be imported by the recombinant yeast host cell. In some embodiments, the yeast cellular components can include carbohydrate residues, amino acid residues, lipidic moieties and/or nucleic acid residues. The yeast cellular component can be, for example, a carbohydrate (DP1, DP2, DP3 or higher), a peptide or a polypeptide (which can be, in some embodiments, bear one or more carbohydrate moieties), a lipid, etc. The yeast cellular component can, in some embodiments, be exported by or imported in the recombinant yeast host cell. The heterologous hydrolase has the ability to decrease the concentration/content of the substrate as well as increase the concentration/content of its associated enzymatic product.

In some embodiments, the yeast cellular component can be an intracellular yeast component, e.g. a component which is located inside the yeast cell. In some embodiments, the intracellular yeast component can be imported by the recombinant yeast host cell. The intracellular yeast component can be physically associated with the cellular membrane, with an organelle and/or with the nucleus. The intracellular yeast component can be located in the cytoplasm of the yeast host cell. The intracellular yeast component can be located in the nucleus of the yeast host cell.

In some embodiments, the yeast cellular component can be associated with the cell membrane of the yeast host cell (e.g., it can be physically associated, directly or indirectly with the cell membrane of the yeast host cell, it can be a membrane polypeptide located, at least in part, inside the cell membrane of the yeast host cell).

In some further embodiments, the yeast cellular component can be associated with the cell wall of the yeast host cell (e.g., it can be physically associated, directly or indirectly with the cell wall of the yeast host cell, it can be a cell wall polypeptide located, at least in part, inside the cell wall of the yeast host cell).

In some embodiments, the cleavage of the substrate and/or the accumulation of the enzymatic product, if generated prior to the fermentation, can be detrimental (e.g., reduce) the performance of the recombinant yeast host cell during the fermentation. It can reduce, for example, the fermentation yield and/or sugar consumption of the recombinant yeast host cell. It can also increase, for example, the accumulation of unwanted fermentation by-products (such as, for example, glycerol).

The hydrolase can, in some embodiments, be capable (e.g., have the ability) of modifying the enzymatic product into an inhibitory product detrimental to the performance of the recombinant yeast host cell during the fermentation, if generated prior to the fermentation. The presence of the inhibitory product can reduce, for example, the fermentation yield and/or sugar consumption. The presence of the inhibitory product can also increase, for example, the accumulation of unwanted fermentation by-products (such as, for example, glycerol).

In some embodiments, the hydrolase can be a glycoside hydrolase. In some embodiments, the glycoside hydrolase has the ability to use glycogen as a substrate. In some further embodiment, the glycoside hydrolase has the ability of converting an unfermentable carbohydrate source into a fermentable carbohydrate source. As used in the context of the present disclosure, an “unfermentable carbohydrate source” refers to a carbohydrate which cannot be used directly by the recombinant yeast host cell to make a fermentation product. Unfermentable carbohydrate sources must necessarily be hydrolyzed in order to be used to make a fermentation product. By the same token, and still in the context of the present disclosure, a “fermentable carbohydrate source” refers to a carbohydrate which can be used directly by the recombinant yeast host cell to make a the fermentation product. Examples of unfermentable carbohydrate sources include, but are not limited to, a disaccharide (DP2, such as trehalose), a trisaccharide (DP3 such as maltose) or another polysaccharide (DP4+). Example of a fermentable carbohydrate source can be, for example, glucose.

The recombinant yeast host cells of the present disclosure can lack the ability, prior to the introduction of the first heterologous nucleic acid encoding the first heterologous polypeptide or in the absence of expression of the first heterologous polypeptide, to exhibit hydrolase activity and, in some embodiments, to convert the unfermentable carbohydrate source into a fermentable carbohydrate source. In such embodiment, the expression of the first heterologous polypeptide provides the only source of hydrolase enzymatic activity to the recombinant yeast host cell to convert, in some embodiments, the unfermentable carbohydrate source into a fermentable carbohydrate source. Alternatively, the recombinant yeast host cells of the present disclosure have some limited ability, prior to the introduction of the first heterologous nucleic acid encoding the first heterologous polypeptide or in the absence of expression of the first heterologous polypeptide, exhibit hydrolase activity and, in some embodiments, to convert the unfermentable carbohydrate source into a fermentable carbohydrate source. In such embodiment, the expression of the first heterologous polypeptide provides the major source of hydrolase enzymatic activity to the recombinant yeast host cell to, in some embodiments, convert the unfermentable carbohydrate source into a fermentable carbohydrate source.

For example, in most Saccharomyces cerevisiae strains, trehalose cannot be used directly by the recombinant yeast host cell to make a fermentation product (ethanol for example), it must be enzymatically hydrolyzed first and is therefore considered to be an unfermentable carbohydrate source. In the presence of a trehalase (which can be recombinantly expressed in Saccharomyces cerevisiae), trehalose can be hydrolyzed into glucose (e.g., a fermentable carbohydrate source) and the latter can then be used directly to make ethanol.

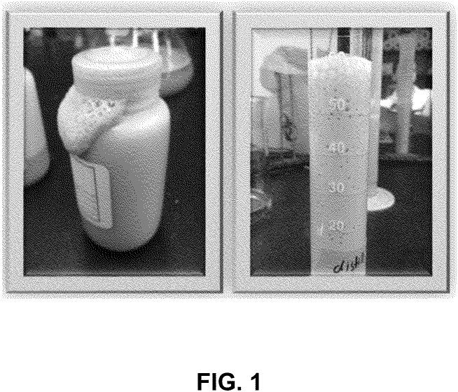

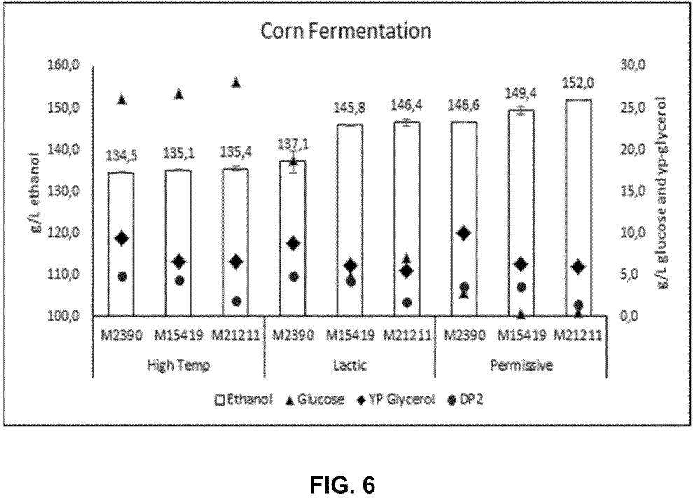

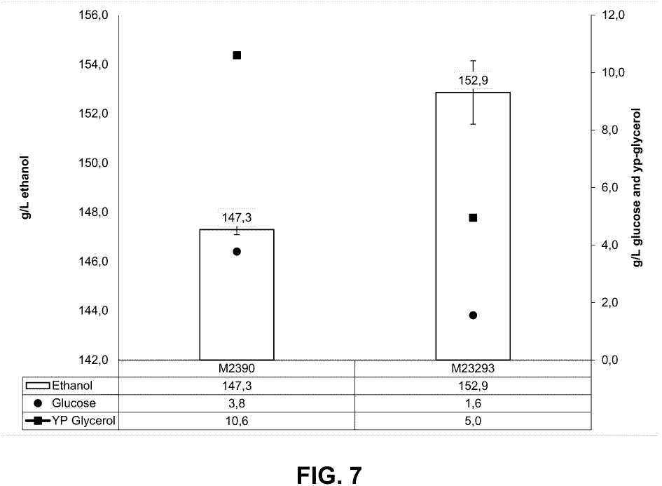

It was determined in the Examples of the present disclosure that recombinant yeast host cells having expressed a first heterologous polypeptide (e.g., a glycoside hydrolase) prior to fermentation (for example during the propagation phase) exhibited instability during storage (e.g., reduction in the number of cells, reduction in the intracellular trehalose content, increase in the production of a fermentation product). This is specifically shown at least in FIG. 1 which illustrates that a recombinant yeast host cell having expressed a heterologous trehalase during the propagation phase, upon storage and prior to fermentation, had begun a fermentation (presumably by converting the trehalose into glucose). This instability during storage can lead to a reduction in fermentation performances in the yeast (e.g., reduction in ethanol production, reduction in glucose and glucose consumption and/or increase in glycerol production, as shown in FIGS. 6 and 7). The recombinant yeast host cell of the present disclosure can be used to promote stability and, in some embodiments, limit fermentation during storage (that may be required after propagation and before fermentation). In specific embodiments, the recombinant yeast host cell of the present disclosure can be used to limit the consumption of a fermentable carbohydrate, the production of a fermentation product (like CO₂ and/or ethanol which can cause foaming) or the production of a fermentation by-product (like glycerol) during storage.

In additional embodiments, the hydrolase can be a peptide hydrolase. In such embodiment, the substrate can be a polypeptide or a peptide. In some specific embodiments, the hydrolase can be a protease. As it was also shown in the Examples below, the expression of protease preferably during fermentation (and limited during propagation) improved the fermentation yield.

In further embodiments, the hydrolase can be a glucan hydrolase. In such embodiment, the substrate can be a glucan (such as, for example β-glucan). In some specific embodiments, the hydrolase can be a glucanase.

### Recombinant Yeast Host Cell and Genetic Modifications

The present disclosure concerns recombinant yeast host cells. The recombinant yeast host cell are obtained by introducing at least one genetic modification in a corresponding ancestral or native yeast host cell. The genetic modifications in the recombinant yeast host cell of the present disclosure comprise, consist essentially of or consist of a first heterologous polypeptide under the control of a heterologous promoter. In the context of the present disclosure, the expression “the genetic modifications in the recombinant yeast host consist essentially of a first genetic modification” refers to the fact that the recombinant yeast host cell can include other genetic modifications which are unrelated or not directly related to the expression of a polypeptide having hydrolase activity.

The genetic modifications of the present disclosure can be aimed at expressing a heterologous polypeptide. In some embodiments, the genetic modification comprises introducing one or more heterologous nucleic acid molecule encoding the heterologous polypeptide. When expressed in a recombinant yeast host cell, the polypeptides described herein are encoded on one or more heterologous nucleic acid molecule. The term “heterologous” when used in reference to a nucleic acid molecule (such as a promoter or a coding sequence) refers to a nucleic acid molecule that is not natively found in the recombinant host cell. “Heterologous” also includes a native coding region, or portion thereof, that is removed from the source organism and subsequently reintroduced into the source organism in a form that is different from the corresponding native gene, e.g., not in its natural location in the organism’s genome. The term “heterologous” when used in reference to a polypeptide refers to a polypeptide which is expressed from the heterologous nucleic acid molecule. The heterologous nucleic acid molecule is purposively introduced into the recombinant host cell. The term “heterologous” as used herein also refers to an element (nucleic acid or polypeptide) that is derived from a source other than the endogenous source. Thus, for example, a heterologous element could be derived from a different strain of host cell, or from an organism of a different taxonomic group (e.g., different kingdom, phylum, class, order, family genus, or species, or any subgroup within one of these classifications). The term “heterologous” is also used synonymously herein with the term “exogenous”.

When a heterologous nucleic acid molecule is present in the recombinant yeast host cell, it can be integrated in the yeast host cell’s genome. The term “integrated” as used herein refers to genetic elements that are placed, through molecular biology techniques, into the genome of a host cell. For example, genetic elements can be placed into the chromosomes of the host cell as opposed to in a vector such as a plasmid or an artificial chromosome carried by the host cell. Methods for integrating genetic elements into the genome of a host cell are well known in the art and include homologous recombination. The heterologous nucleic acid molecule can be present in one or more copies in the yeast host cell’s genome. Alternatively, the heterologous nucleic acid molecule can be independently replicating from the host cell’s genome. In such embodiment, the nucleic acid molecule can be stable and self-replicating.

In some embodiments, heterologous nucleic acid molecules which can be introduced into the recombinant yeast host cells are codon-optimized with respect to the intended recipient recombinant yeast host cell. As used herein the term “codon-optimized coding region” means a nucleic acid coding region that has been adapted for expression in the cells of a given organism by replacing at least one, or more than one, codons with one or more codons that are more frequently used in the genes of that organism. In general, highly expressed genes in an organism are biased towards codons that are recognized by the most abundant tRNA species in that organism. One measure of this bias is the “codon adaptation index” or “CAI,” which measures the extent to which the codons used to encode each amino acid in a particular gene are those which occur most frequently in a reference set of highly expressed genes from an organism. The CAI of codon optimized heterologous nucleic acid molecule described herein corresponds to between about 0.8 and 1.0, between about 0.8 and 0.9, or about 1.0.

The heterologous nucleic acid molecules of the present disclosure comprise a coding region for the one or more polypeptides (including enzymes) to be expressed by the recombinant host cell. A DNA or RNA “coding region” is a DNA or RNA molecule which is transcribed and/or translated into a polypeptide in a cell in vitro or in vivo when placed under the control of appropriate regulatory sequences. “Suitable regulatory regions” refer to nucleic acid regions located upstream (5′ non-coding sequences), within, or downstream (3′ non-coding sequences) of a coding region, and which influence the transcription, RNA processing or stability, or translation of the associated coding region. Regulatory regions may include promoters, translation leader sequences, RNA processing sites, effector binding sites and stem-loop structures. The boundaries of the coding region are determined by a start codon at the 5′ (amino) terminus and a translation stop codon at the 3′ (carboxyl) terminus. A coding region can include, but is not limited to, prokaryotic regions, cDNA from mRNA, genomic DNA molecules, synthetic DNA molecules, or RNA molecules. If the coding region is intended for expression in a eukaryotic cell, a polyadenylation signal and transcription termination sequence will usually be located 3′ to the coding region. In an embodiment, the coding region can be referred to as an open reading frame. “Open reading frame” is abbreviated ORF and means a length of nucleic acid, either DNA, cDNA or RNA, that comprises a translation start signal or initiation codon, such as an ATG or AUG, and a termination codon and can be potentially translated into a polypeptide sequence.

The heterologous nucleic acid molecules described herein can comprise a non-coding region, for example a transcriptional and/or translational control regions. “Transcriptional and translational control regions” are DNA regulatory regions, such as promoters, enhancers, terminators, and the like, that provide for the expression of a coding region in a host cell. In eukaryotic cells, polyadenylation signals are control regions.

The heterologous nucleic acid molecule can be introduced and optionally maintained in the host cell using a vector. A “vector,” e.g., a “plasmid”, “cosmid” or “artificial chromosome” (such as, for example, a yeast artificial chromosome) refers to an extra chromosomal element and is usually in the form of a circular double-stranded DNA molecule. Such vectors may be autonomously replicating sequences, genome integrating sequences, phage or nucleotide sequences, linear, circular, or supercoiled, of a single- or double-stranded DNA or RNA, derived from any source, in which a number of nucleotide sequences have been joined or recombined into a unique construction which is capable of introducing a promoter fragment and DNA sequence for a selected gene product along with appropriate 3′ untranslated sequence into a host cell.

The promoter of the present disclosure have the ability to control (e.g., limit, allow or favor) the expression of the nucleic acid molecule to which it is operatively linked to. In the context of the present disclosure, the expressions “operatively linked” or “operatively associated” refers to fact that the promoter is physically associated to the nucleotide acid molecule coding for the one or more enzyme in a manner that allows, under certain conditions, for expression of the one or more enzyme from the nucleic acid molecule. In an embodiment, the promoter can be located upstream (5′) of the nucleic acid sequence coding for the one or more enzyme. In still another embodiment, the promoter can be located downstream (3′) of the nucleic acid sequence coding for the one or more enzyme. In the context of the present disclosure, one or more than one promoter can be included in the heterologous nucleic acid molecule. When more than one promoter is included in the heterologous nucleic acid molecule, each of the promoters is operatively linked to the nucleic acid sequence coding for the one or more enzyme. The promoters can be located, in view of the nucleic acid molecule coding for the one or more polypeptide, upstream, downstream as well as both upstream and downstream.

“Promoter” refers to a DNA fragment capable of controlling the expression of a coding sequence or functional RNA. The term “expression,” as used herein, refers to the transcription and stable accumulation of sense (mRNA) from the heterologous nucleic acid molecule described herein. Expression may also refer to translation of mRNA into a polypeptide. Promoters may be derived in their entirety from a native gene, or be composed of different elements derived from different promoters found in nature, or even comprise synthetic DNA segments. It is understood by those skilled in the art that different promoters may direct the expression at different stages of development, or in response to different environmental or physiological conditions. Promoters which cause a gene to be expressed in most cells at most times at a substantial similar level are commonly referred to as “constitutive promoters”. It is further recognized that since in most cases the exact boundaries of regulatory sequences have not been completely defined, DNA fragments of different lengths may have identical promoter activity. A promoter is generally bounded at its 3′ terminus by the transcription initiation site and extends upstream (5′ direction) to include the minimum number of bases or elements necessary to initiate transcription at levels detectable above background. Within the promoter will be found a transcription initiation site (conveniently defined for example, by mapping with nuclease S1), as well as polypeptide binding domains (consensus sequences) responsible for the binding of the polymerase.

One or more promoters can be used to allow the expression of each heterologous polypeptides in the recombinant yeast host cell. In the context of the present disclosure, the expression “functional fragment of a promoter” when used in combination to a promoter refers to a shorter nucleic acid sequence than the native promoter which retain the ability to control the expression of the nucleic acid sequence encoding the heterologous polypeptide during the propagation phase of the recombinant yeast host cells. Usually, functional fragments are either 5′ and/or 3′ truncation of one or more nucleic acid residue from the native promoter nucleic acid sequence.

The promoter can be heterologous to the nucleic acid molecule encoding the one or more polypeptides. The promoter can be heterologous or derived from a strain being from the same genus or species as the recombinant yeast host cell. In an embodiment, the promoter is derived from the same genus or species of the yeast host cell and the heterologous polypeptide is derived from different genus that the host cell. In an embodiment, the promoter used in the heterologous nucleic acid molecule is the same promoter that controls the expression of the encoded polypeptide in its native context.

In an embodiment, the present disclosure concerns the expression of one or more polypeptide (including an enzyme), a variant thereof or a fragment thereof in a recombinant host cell. A variant polypeptide comprises at least one amino acid residue difference when compared to the amino acid sequence of the native polypeptide (enzyme) and exhibits a biological activity substantially similar to the native polypeptide. A variant nucleic acid molecule comprises at least one nucleic acid residue difference when compared to the nucleic acid sequence of the native nucleic acid molecule and exhibits a biological activity substantially similar to the native nucleic acid molecule. The “variants” have at least 50%, 55%, 60%, 65%, 70%, 75%, 80%, 85%, 90%, 95%, 96%, 97%, 98% or 99% identity to the polypeptide or the nucleic acid molecule described herein. The heterologous “variants” can have at least 50%, 55%, 60%, 65%, 70%, 75%, 80%, 85%, 90%, 95%, 96%, 97%, 98% or 99% biological activity when compared to the native polypeptide or the native nucleic acid molecule described herein. The term “percent identity”, as known in the art, is a relationship between two or more polypeptide sequences or two or more polynucleotide sequences, as determined by comparing the sequences. The level of identity can be determined conventionally using known computer programs. Identity can be readily calculated by known methods, including but not limited to those described in: Computational Molecular Biology (Lesk, A. M., ed.) Oxford University Press, NY (1988); Biocomputing: Informatics and Genome Projects (Smith, D. W., ed.) Academic Press, NY (1993); Computer Analysis of Sequence Data, Part I (Griffin, A. M., and Griffin, H. G., eds.) Humana Press, NJ (1994); Sequence Analysis in Molecular Biology (von Heinje, G., ed.) Academic Press (1987); and Sequence Analysis Primer (Gribskov, M. and Devereux, J., eds.) Stockton Press, NY (1991). Preferred methods to determine identity are designed to give the best match between the sequences tested. Methods to determine identity and similarity are codified in publicly available computer programs. Sequence alignments and percent identity calculations may be performed using the Megalign program of the LASERGENE bioinformatics computing suite (DNASTAR Inc., Madison, Wis.). Multiple alignments of the sequences disclosed herein were performed using the Clustal method of alignment (Higgins and Sharp (1989) CABIOS. 5:151-153) with the default parameters (GAP PENALTY=10, GAP LENGTH PEN ALT Y= 10). Default parameters for pairwise alignments using the Clustal method were KTUPLB 1, GAP PENALTY=3, WINDOW=5 and DIAGONALS SAVED=5.

The variant polypeptide described herein may be (i) one in which one or more of the amino acid residues are substituted with a conserved or non-conserved amino acid residue (preferably a conserved amino acid residue) and such substituted amino acid residue may or may not be one encoded by the genetic code, or (ii) one in which one or more of the amino acid residues includes a substituent group, or (iii) one in which the mature polypeptide is fused with another compound, such as a compound to increase the half-life of the polypeptide (for example, polyethylene glycol), or (iv) one in which the additional amino acids are fused to the mature polypeptide for purification of the polypeptide.

A “variant” of the polypeptide can be a conservative variant or an allelic variant. As used herein, a conservative variant refers to alterations in the amino acid sequence that do not adversely affect the biological functions of the polypeptide/enzyme. A substitution, insertion or deletion is said to adversely affect the polypeptide when the altered sequence prevents or disrupts a biological function associated with the enzyme. For example, the overall charge, structure or hydrophobic-hydrophilic properties of the polypeptide can be altered without adversely affecting a biological activity. Accordingly, the amino acid sequence can be altered, for example to render the polypeptide more hydrophobic or hydrophilic, without adversely affecting the biological activities of the enzyme.

The polypeptide can be a fragment of the polypeptide or fragment of the variant polypeptide. A polypeptide fragment comprises at least one less amino acid residue when compared to the amino acid sequence of the possesses and still possess a biological activity substantially similar to the native full-length polypeptide or polypeptide variant. In some embodiments, the fragment can correspond to the polypeptide amino acid sequence in which the native signal sequence has been removed (and optionally replaced by another signal sequence). The nucleic acid molecule can be a fragment of the nucleic acid molecule or fragment of the variant nucleic acid molecule. A nucleic acid molecule fragment comprises at least one less nucleic acid residue when compared to the nucleic acid sequence of the possesses and still possess a biological activity substantially similar to the native full-length nucleic acid molecule or nucleic acid molecule variant. In some embodiments, the fragment can correspond to the nucleic acid sequence in which the sequence encoding the signal sequence has been removed (and optionally replaced by another sequence encoding another signal sequence). Polypeptide “fragments” can have at least at least 100, 200, 300, 400, 500 or more consecutive amino acids of the polypeptide or the polypeptide variant. The polypeptide and nucleic acid molecule “fragments” can have at least 50%, 55%, 60%, 65%, 70%, 75%, 80%, 85%, 90%, 95%, 96%, 97%, 98% or 99% identity to the polypeptide, the variant polypeptide, the nucleic acid molecule or the variant nucleic acid molecule. The polypeptide and the nucleic acid molecule “fragments” can have at least 50%, 55%, 60%, 65%, 70%, 75%, 80%, 85%, 90%, 95%, 96%, 97%, 98% or 99% biological activity when compared to the polypeptide, the variant polypeptide, the nucleic acid molecule or the variant nucleic acid molecule. In some embodiments, fragments of the polypeptides can be employed for producing the corresponding full-length enzyme by peptide synthesis. Therefore, the fragments can be employed as intermediates for producing the full-length polypeptides.

In some additional embodiments, the present disclosure also provides expressing a polypeptide encoded by a gene ortholog of a gene known to encode the polypeptide. A “gene ortholog” is understood to be a gene in a different species that evolved from a common ancestral gene by speciation. In the context of the present disclosure, a gene ortholog encodes polypeptide exhibiting a biological activity substantially similar to the native polypeptide.

In some further embodiments, the present disclosure also provides expressing a polypeptide encoded by a gene paralog of a gene known to encode the polypeptide. A “gene paralog” is understood to be a gene related by duplication within the genome. In the context of the present disclosure, a gene paralog encodes a polypeptide that could exhibit additional biological functions when compared to the native polypeptide.

Additional genetic modifications can also be included in the recombinant yeast host cell for reducing or inhibiting the expression of a specific targeted gene (which is endogenous to the host cell). In such instances, the genetic modifications can be made in one or both copies of the targeted gene(s). When the genetic modification is aimed at increasing the expression of a specific targeted gene, the genetic modification can be made in one or multiple genetic locations. In the context of the present disclosure, when recombinant yeast host cells are qualified as being “genetically engineered”, it is understood to mean that they have been manipulated to either add at least one or more heterologous or exogenous nucleic acid residue and/or remove at least one endogenous (or native) nucleic acid residue. In some embodiments, the one or more nucleic acid residues that are added can be derived from a heterologous cell or the recombinant yeast host cell itself. In the latter scenario, the nucleic acid residue(s) is (are) added at a genomic location which is different than the native genomic location. The genetic manipulations did not occur in nature and are the results of in vitro manipulations of the native yeast host cell.

In the context of the present disclosure, the recombinant/native host cell is a yeast. Suitable yeast host cells can be, for example, from the genus Saccharomyces, Kluyveromyces, Arxula, Debaryomyces, Candida, Pichia, Phaffia, Schizosaccharomyces, Hansenula, Kloeckera, Schwanniomyces or Yarrowia. Suitable yeast species can include, for example, S. cerevisiae, S. bulderi, S. barnetti, S. exiguus, S. uvarum, S. diastaticus, K. lactis, K. marxianus or K. fragilis. In some embodiments, the yeast is selected from the group consisting of Saccharomyces cerevisiae, Schizzosaccharomyces pombe, Candida albicans, Pichia pastoris, Pichia stipitis, Yarrowia lipolytica, Hansenula polymorpha, Phaffia rhodozyma, Candida utilis, Arxula adeninivorans, Debaryomyces hansenii, Debaryomyces polymorphus, Schizosaccharomyces pombe and Schwanniomyces occidentalis. In one particular embodiment, the yeast is Saccharomyces cerevisiae. In some embodiments, the host cell can be an oleaginous yeast cell. For example, the oleaginous yeast host cell can be from the genus Blakeslea, Candida, Cryptococcus, Cunninghamella, Lipomyces, Mortierella, Mucor, Phycomyces, Pythium, Rhodosporidum, Rhodotorula, Trichosporon or Yarrowia. In some alternative embodiments, the host cell can be an oleaginous microalgae host cell (e.g., for example, from the genus Thraustochytrium or Schizochytrium). In an embodiment, the recombinant yeast host cell is from the genus Saccharomyces and, in some additional embodiments, from the species Saccharomyces cerevisiae.

Since the recombinant yeast host cell can be used for the fermentation of a biomass and the generation of fermentation product, it is contemplated herein that it has the ability to convert a biomass into a fermentation product without including the additional genetic modifications described herein. In an embodiment, the recombinant yeast host cell has the ability to convert starch into ethanol during fermentation, as it is described below. In still another embodiment, the recombinant yeast host cell of the present disclosure can be genetically modified to provide or increase the biological activity of one or more polypeptide involved in the fermentation of the biomass and the generation of the fermentation product.

### First Heterologous Polypeptide

The recombinant yeast host cell of the present disclosure has the ability to express a first heterologous polypeptide. The first heterologous polypeptide refers an enzyme (or a combination of enzymes) exhibiting hydrolase activity. Hydrolases define a class of enzymes capable of catalyzing the breakage of a chemical bond by using water. Hydrolases are classified as EC 3 in the EC number classification of enzymes. Hydrolases can be further classified into several subclasses, based upon the bonds they act upon: EC 3.1: ester bonds (esterases: nucleases, phosphodiesterases, lipase, phosphatase), EC 3.2: sugars (DNA glycosylases, glycoside hydrolase), EC 3.3: ether bonds, EC 3.4: peptide bonds (Proteases/peptidases), EC 3.5: carbon-nitrogen bonds, other than peptide bonds, EC 3.6 acid anhydrides (acid anhydride hydrolases, including helicases and GTPase), EC 3.7 carbon-carbon bonds, EC 3.8 halide bonds, EC 3.9: phosphorus-nitrogen bonds, EC 3.10: sulphur-nitrogen bonds, EC 3.11: carbon-phosphorus bonds, EC 3.12: sulfur-sulfur bonds and EC 3.13: carbon-sulfur bonds. In the context of the present disclosure, the heterologous hydrolase has the ability to convert a substrate, which is a yeast cellular component, in an enzymatic product.

The hydrolase that can be expressed in the recombinant yeast host cell of the present disclosure has the ability to generate an hydrolyzed enzymatic product from a substrate. In some embodiments, the degradation of the substrate, prior to fermentation, can be detrimental to the fermentation performance (e.g., fermentation yield, carbohydrate consumption, and/or gas production) of the recombinant yeast and/or can reduce the stability (e.g., viability, early fermentation) of the recombinant yeast host cell prior to fermentation. In some embodiments, the enzymatic product, if generated prior to fermentation, can be detrimental to the fermentation performance (e.g., fermentation yield, carbohydrate consumption, and/or gas production) of the recombinant yeast as its presence can reduce the stability (e.g., viability, early fermentation) of the recombinant yeast host cell prior to fermentation. In additional embodiments, the reduction in the substrate and the increase in the enzyme product is not directly detrimental to the stability of the recombinant yeast host cell prior to fermentation. However, because, in some embodiments, the recombinant yeast host cell has the ability to further convert the enzymatic product in an inhibitory product (e.g., an alcohol such as, for example, ethanol), it can exhibit a reduction in stability and/or fermentation performance if such inhibitory product is generated prior to fermentation.

The hydrolase can be a phosphatase. As used herein, the expression “phosphatase” refers to a polypeptide having enzymatic activity and capable, in the presence of water, of catalyzing the cleavage of a phosphoric acid monoester into a phosphate ion and an alcohol. An embodiment of a phosphatase is a phytase, a polypeptide having enzymatic activity and capable of catalyzing the hydrolysis of phytic acid (myo-inositol hexakisphosphate) into inorganic phosphorus. There are four distinct classes of phytase: histidine acid phosphatases (HAPS), β-propeller phytases, purple acid phosphatases and protein tyrosine phosphatase-like phytases (PTP-like phytases). Phytic acid has six phosphate groups that may be released by phytases at different rates and in different order. Phytases hydrolyze phosphates from phytic acid in a stepwise manner, yielding products that again become substrates for further hydrolysis. Phytases have been grouped based on the first phosphate position of phytic acid that is hydrolyzed: are 3-phytase (EC 3.1.3.8), 4-phytase (EC 3.1.3.26) and 5-phytase (EC 3.1.3.72). In an embodiment, the phytase is derived from a bacterial species, such as, for example, a Citrobacter sp. or an Escherichia sp. In a specific embodiment, the heterologous phytase is derived from a Citrobacter sp., such as for example Citrobacter braakii. In another embodiment, the heterologous phytase is derived from an Escherichia sp., such as, for example, Escherichia coli. The degradation of phosphate moiety and/or the presence of cleaved phosphate moiety (directly or indirectly from the generation of the inhibitory product), prior to fermentation, can be detrimental to the stability of the yeast as well as its performance of the recombinant yeast host cell during the fermentation. In such embodiments, the substrate/yeast cellular component can comprise a phosphate moiety.

The hydrolase can be an amylolytic enzyme. The expression “amylolytic enzyme” refers to a class of enzymes capable of hydrolyzing starch or hydrolyzed starch. Amylolytic enzymes include, but are not limited to alpha-amylases (EC 3.2.1.1, sometimes referred to fungal alpha-amylase, see below), maltogenic amylase (EC 3.2.1.133), glucoamylase (EC 3.2.1.3), glucan 1,4-alpha-maltotetraohydrolase (EC 3.2.1.60), pullulanase (EC 3.2.1.41), iso-amylase (EC 3.2.1.68) and amylomaltase (EC 2.4.1.25). In an embodiment, the one or more amylolytic enzymes can be an alpha-amylase from Aspergillus oryzae, a maltogenic alpha-amylase from Geobacillus stearothermophilus, a glucoamylase from Saccharomycopsis fibuligera (and in some embodiments, have the amino acid sequence of SEQ ID NO: 1, 32 or 34, be a variant of the amino acid sequence of SEQ ID NO: 1, 32 or 34 or be a fragment of the amino acid sequence of SEQ ID NO: 1, 32 or 34), a glucan 1,4-alpha-maltotetraohydrolase from Pseudomonas saccharophila, a pullulanase from Bacillus naganoensis, a pullulanase from Bacillus acidopullulyticus, an iso-amylase from Pseudomonas amyloderamosa, and/or amylomaltase from Thermus thermophilus. The degradation of starch and/or the presence of cleaved starch (directly or indirectly from the generation of the inhibitory product), prior to fermentation, can be detrimental to the stability of the yeast as well as its performance of the recombinant yeast host cell during the fermentation. In such embodiments, the substrate/yeast cellular component can be starch or a starch-containing biological molecule.

The hydrolase can be a cellulase or an hemi-cellulase. As used herein, the expression “cellulase/hemi-cellulase” refers to a class of enzymes capable of hydrolyzing, respectively, cellulose or hemi-cellulose. Cellulases/hemi-cellulases include, but are not limited to a cellulase (E.C. 3.2.1.4) and an endoB(1,4)D-xylanase (E.C. 3.2.1.8). In an embodiment, the one or more cellulase/hemi-cellulase can be a cellulase from Penicillium funiculosum, an endoB(1,4)D-xylanase from Rasamsonia emersonii and/or a xylanase from Aspergillus niger (which can have the amino acid sequence of SEQ ID NO: 38, be a variant of the amino acid sequence of SEQ ID NO: 38 or a be a fragment of the amino acid sequence of SEQ ID NO: 38). The degradation of cellulose and/or the presence of cleaved cellulose (directly or indirectly from the generation of the inhibitory product), prior to fermentation, can be detrimental to the stability of the yeast as well as its performance of the recombinant yeast host cell during the fermentation. In such embodiments, the substrate/yeast cellular component can be cellulose or hemi-cellulose or a cellulose- or hemi-cellulose-containing biological molecule.

The hydrolase can be a lipase. As used herein, the expression “lipase” refers to a class of enzymes capable of hydrolyzing lipids. In an embodiment, the one or more lipase can be a triacylglycerol lipase from Thermomyces lanuginosis, a phospholipase A2 from Sus scrofa, a phospholipase A2 from Streptomyces vialaceoruber and/or a phospholipase A2 from Aspergillus oryzea. The degradation of a lipid and/or the presence of cleaved lipids (directly or indirectly from the generation of the inhibitory product), prior to fermentation, can be detrimental to the stability of the yeast as well as its performance of the recombinant yeast host cell during the fermentation. In such embodiments, the substrate/yeast cellular component can be a lipid or a lipid-containing biological molecule.

The hydrolase can be a peptide hydrolase, such as, for example, a protease from EC 3.4. In the context of the present disclosure, the term “protease” (also referred to as “peptidase”) refers to a polypeptide having proteolytic activity (e.g., a proteolytic enzyme). Such enzymes can be classified into two groups based on the type of proteolytic activity they exhibit: endopeptidases (which include proteinases) and exopeptidases. Endopeptidases exhibit endo-acting peptide bond hydrolase activity, whereas exopeptidases exhibit exo-acting peptide bond hydrolase activity. Proteases can also be classified according to their catalytic residue: serine proteases (using a serine alcohol), cysteine proteases (using a cysteine thiol), threonine proteases (using a threonine secondary alcohol), aspartic proteases (using an aspartate carboxylic acid), glutamic proteases (using a glutamate carboxylic acid), metalloproteases (using a metal) and asparagine peptide lyases (using an asparagine to perform an elimination reaction). Proteases can also be classified according to their optimal pH (e.g., the pH at which the protease has the most enzymatic activity). The recombinant yeast host cell can express a heterologous protease which is neutral or acidic. When the optimal pH of a protease is neutral (e.g., between pH 6.0 and 7.5), the protease is considered to be a neutral protease. When the optimal pH of a protease is acidic (e.g., below 6.0), the protease is considered to be an acidic protease. In some embodiments, an acidic protease has an optimal pH between 2.0 and 5.0 and is inactivated at a pH above 6.0. It is understood that since the yeast fermentation is conducted at an acidic pH (e.g., between 4.0 to 5.5 for example) it may be advantageous that the recombinant yeast host cell expresses a neutral or acidic protease (which may be native or heterologous) to increase its proteolytic activity. Proteases are able to cleave polypeptides or peptides (e.g., its substrate) to generate smaller amino acid chains or amino acid residues (e.g., its products). The degradation of polypeptides/peptides and/or the presence of cleaved polypeptides (directly or indirectly from the generation of the inhibitory product), prior to fermentation, can be detrimental to the stability of the yeast as well as its performance of the recombinant yeast host cell during the fermentation. In such embodiments, the substrate/yeast cellular component can be a peptide or a polypeptide or a peptide- or polypeptide-containing biological molecule.

In a specific embodiment, the heterologous protease is a secreted and extracellular protease such as for example a member of the subtilisin serine protease family. In an embodiment, the member of the subtilisin serine protease family is a cell-envelop proteinase (CEP). In an embodiment, the CEP can be, for example, lactocepin (which may also be referred to as PrtP), PrtB, PrtH, PrtR or PrtS.

Lactocepin is encoded by the prtP gene. Lactocepin is an extracellular protease which exhibits endopeptidase activity and is associated with the bacterial cell envelop. It is attached to the bacterial cell envelop with a LPxTG motif. In an embodiment, the lactocepin is derived from Lactobacillus sp., for example a Lactobacillus paracasei. In an embodiment, the heterologous protease is associated with GenBank accession number WP_014952255, is a variant of GenBank accession number WP_014952255 or is a fragment of GenBank accession number WP_014952255. In another embodiment, the lactocepin is derived from a Lactococus sp., for example a Lactococcus lactis. In another embodiment, the heterologous protease is associated with GenBank accession number ARE27274 , is a variant of GenBank accesion number ARE27274 or is a fragment of GenBank accession number ARE27274.

PrtB is encoded by the prtB gene. PrtB is an extracellular protease which exhibits endopeptidase activity and is associated with the bacterial cell envelop. It is attached to the bacterial cell envelop with a LPxTG motif. In an embodiment, PrtB is derived from Lactobacillus sp., for example a Lactobacillus delbrueckii. In an embodiment, the heterologous protease is associated with GenBank accession number EPB98635, is a variant of GenBank accession number EPB98635 or is a fragment of GenBank accession number EPB98635.

PrtH is encoded by the prtH gene. PrtH is an extracellular protease which exhibits endopeptidase activity and is associated with the bacterial cell envelop. It is attached to the bacterial cell envelop with a LPxTG motif. In an embodiment, PrtH is derived from Lactobacillus sp., for example a Lactobacillus helveticus. In an embodiment, the heterologous protease is associated with GenBank accession number AAD50643, is a variant of GenBank accession number AAD50643 or is a fragment of GenBank accession number AAD50643.

PrtR is encoded by the prtR gene. PrtR is an extracellular protease which exhibits endopeptidase activity and is associated with the bacterial cell envelop. It is attached to the bacterial cell envelop with a LPxTG motif. In an embodiment, PrtR is derived from Lactobacillus sp., for example a Lactobacillus rhamnosus. In an embodiment, the heterologous protease is associated with GenBank accession number CAD43138, is a variant of GenBank accession number CAD43138 or is a fragment of GenBank accession number CAD43138.

PrtS is encoded by the prtS gene. PrtS is an extracellular protease which exhibits endopeptidase activity and is associated with the bacterial cell envelop. It is attached to the bacterial cell envelop with a LPxTG motif. In an embodiment, PrtS is derived from Steptococcus sp., for example a Streptococcus thermophilus. In an embodiment, the heterologous protease is associated with GenBank accession number BBQ09553, is a variant of GenBank accession number BBQ09553 or is a fragment of GenBank accession number BBQ09553.

PepN is an intracellular exopeptidase which can, in some embodiments, be expressed by the recombinant bacterial cell. In an embodiment, PepN is derived from a Lactobacillus sp. In yet additional embodiments, PepN is derived from Lactobacillus helveticus and even can be associated with GenBank accession number AGQ22917, is a variant of AGQ22917 or is a fragment of AGQ22917. In yet additional embodiments, PepN is derived from Lactobacillus casei and even can be associated with GenBank accession number GEK39407, is a variant of GEK39407 or is a fragment of GEK39407. In an embodiment, PepN is derived from a Lactococcus sp. In yet additional embodiments, PepN is derived from Lactococcus lactis and even can be associated with GenBank accession number CAL96925.1, is a variant of CAL96925.1 or is a fragment of CAL96925.1.

In yet another embodiment, the heterologous protease can be a secreted and extracellular metalloprotease (including a zinc-dependent metalloprotease). In an embodiment, the secreted and extracellular metalloprotease can be derived from Bacillus sp., for example from Bacillus subtilis or from Bacillus thermoproteolyticus. In an embodiment, the metalloprotease can be NprE. NprE is encoded by the nprE gene. In an embodiment, NprE is derived from a Bacillus sp., for example a Bacillus subtilis. In an embodiment, the heterologous protease is associated with GenBank accession number WP_168780890, is a variant of GenBank accession number WP_168780890 or is a fragment of GenBank accession number WP_168780890. In another embodiment, the secreted and extracellular metalloprotease can be from the peptidase family M4 (thermolysin) family. In an embodiment, NprE is derived from a Bacillus sp., for example a Bacillus subtilis. In an embodiment, the heterologous protease is associated with GenBank accession number CAA54291, is a variant of GenBank accession number CAA54291 or is a fragment of GenBank accession number CAA54291.

In an embodiment, the heterologous protease is an aspartic protease or a protease susceptible of having aspartic-like activity. The heterologous protease can be derived from a known protease expressed in a prokaryotic (such as a bacteria) or a eukaryotic cell (such as a yeast, a mold, a plant or an animal). Embodiments of aspartic proteases include, without limitation, SAP1 (from Candida albicans or from Candida dubliniensis), PEP1 (from Aspergillus fumigatus or from Saccharomycopsis fibuligera)

In an embodiment, the heterologous protease can be derived from a fungal organism. For example, the heterologous protease can be derived from the genus Candida, Clavispora, Saccharomyces, Yarrowia, Meyerozyma, Aspergillus or Saccharomycopsis. When the heterologous protease is derived from the genus Candida, it can be derived from the species Candida albicans, Candida dubliniensis or Candida tropicalis. When the heterologous protease is derived from Candida albicans, it can have the amino acid of SEQ ID NO: 37, be a variant of the amino acid of SEQ ID NO: 37 or be a fragment of SEQ ID NO: 37. When the heterologous protease is derived from Candida dubliensis, it can have the amino acid sequence of SEQ ID NO: 46, be a variant of SEQ ID NO: 46, or be a fragment of SEQ ID NO: 46. When the heterologous protease is derived from Candida tropicalis, it can have the amino acid sequence of SEQ ID NO: 47, be a variant of SEQ ID NO: 47, or be a fragment of SEQ ID NO: 47. When the heterologous protease is derived from the genus Clavispora, it can be derived from the species Clavispora lusitaniae. When the heterologous protease is derived from the species Clavispora lusitaniae, it can have the amino acid sequence of SEQ ID NO: 48 or 49, be a variant of the amino acid sequence of SEQ ID NO: 48 or 49, or be a fragment of the amino acid sequence of SEQ ID NO: 48 or 49. When the heterologous protease is derived from the genus Saccharomyces, it can be derived from the species Saccharomyces cerevisiae. When the heterologous protease is derived from the species Saccharomyces cerevisiae, it can have the amino acid sequence of SEQ ID NO: 50, be a variant of the amino acid sequence of SEQ ID NO: 50 or be a fragment of the amino acid sequence of SEQ ID NO: 50. When the heterologous protease is derived from the genus Yarrowia, it can be derived from the species Yarrowia lipolytica. When the heterologous protease is derived from the species Yarrowia lipolytica, it can have the amino acid sequence of SEQ ID NO: 51, be a variant of the amino acid sequence of SEQ ID NO: 51 or be a fragment of the amino acid sequence of SEQ ID NO: 51. When the heterologous protease is derived from the genus Meyerozyma, it can be derived from the species Meyerozyma guilliermondii. When the heterologous protease is derived from the species Meyerozyma guilliermondii, it can have the amino acid sequence of SEQ ID NO: 52, be a variant of the amino acid sequence of SEQ ID NO: 52 or be a fragment of the amino acid sequence of SEQ ID NO: 52. When the heterologous protease is derived from the genus Aspergillus, it can be derived from the species Aspergillus fumigatus. When the heterologous protease is derived from the species Aspergillus fumigatus, it can have the amino acid sequence of SEQ ID NO: 53, be a variant of the amino acid sequence of SEQ ID NO: 53 or be a fragment of the amino acid sequence of SEQ ID NO: 53. When the heterologous protease is derived from the species Saccharomycopsis, it can be derived from the species Saccharomycopsis fibuligera. When the heterologous protease is derived from the species Saccharomycopsis fibuligera, it can have the amino acid sequence of SEQ ID NO: 54, be a variant of the amino acid sequence of SEQ ID NO: 54 or be a fragment of the amino acid sequence of SEQ ID NO: 54.

In an embodiment, the heterologous protease can be derived from a bacterial organism. For example, the heterologous protease can be derived from the genus Bacillus. When the heterologous protease is derived from the genus Bacillus, it can be derived from the species Bacillus subtilis, it can have the amino acid sequence of SEQ ID NO: 55, be a variant of the amino acid sequence of SEQ ID NO: 55 or be a fragment of the amino acid sequence of SEQ ID NO: 55.

In an embodiment, the heterologous protease can be derived from a plant. For example, the heterologous protease can be derived from the genus Ananas. When the heterologous protease is derived from the genus Ananas, it can be derived from the species Ananas comosus, it can have the amino acid sequence of SEQ ID NO: 56, be a variant of the amino acid sequence of SEQ ID NO: 56 or be a fragment of the amino acid sequence of SEQ ID NO: 56.

The hydrolase can be a glucan hydrolase, such as, for example a glucanase from EC 3.2. Glucanases are able to cleave glucan, a glucose polymer, into shorter saccharide chains or even monosaccharides. Glucanase can cleave α bonds or β bonds which may be present in a glucan. In an embodiment, the glucanase is a β glucanase. The degradation of glucans and/or the presence of cleaved glucan moieties (directly or indirectly from the generation of the inhibitory product), prior to fermentation, can be detrimental to the stability of the yeast as well as its performance of the recombinant yeast host cell during the fermentation. In such embodiments, the substrate/yeast cellular component can be glucan (such as β glucan) or a glucan (or β glucan)-containing biological molecule.

The hydrolase can be a glycoside hydrolase from EC 3.2. Glycoside hydrolases are able to cleave carbohydrate chains (e.g., its substrate) to generate smaller carbohydrates chains or discrete carbohydrate molecules (e.g., its products). The degradation of carbohydrate chains and/or the presence of cleaved carbohydrate chains (directly or indirectly from the generation of the inhibitory product), prior to fermentation, can be detrimental to the stability of the yeast as well as its performance of the recombinant yeast host cell during the fermentation. In such embodiments, the substrate/yeast cellular component can be a carbohydrate chain or a carbohydrate-containing biological molecule.

In some embodiment, the glycoside hydrolase is capable of converting an unfermentable carbohydrate source (for example a disaccharide, a trisaccharide or a polysaccharide) into a fermentable carbohydrate source (for example a monosaccharide). In some specific embodiments, the glycoside hydrolase can be a trehalase capable of converting an unfermentable carbohydrate source (trehalose) into a fermentable carbohydrate source (glucose). In such embodiments, the substrate/yeast cellular component can be trehalose or a trehalose-containing biological molecule.

In a specific embodiment, the first heterologous polypeptide is a glucoside hydrolase capable of hydrolyzing an unfermentable carbohydrate source that is present in the storage medium (e.g., trehalose for example). The first heterologous polypeptide can have trehalase activity and can be a trehalase. Trehalases are glycoside hydrolases capable of converting trehalose into glucose. Trehalases have been classified under EC number 3.2.1.28. Trehalases can be classified into two broad categories based on their optimal pH: neutral trehalases (having an optimum pH of about 7) and acid trehalases (having an optimum pH of about 4.5). The heterologous trehalases that can be used in the context of the present disclosure can be of various origins such as bacterial, fungal or plant origin. In a specific embodiment, the trehalase is from fungal origin. In such embodiment, the substrate or cellular component can be trehalose or a trehalose-containing biological product.

In an embodiment, the trehalase is from Aspergillus sp., for example Aspergillus fumigatus which can have, in some embodiments, the amino acid sequence of SEQ ID NO: 14, be a variant of the amino acid sequence of SEQ ID NO: 14 or be a fragment of the amino acid sequence of SEQ ID NO: 14. In an embodiment, the trehalase is from Neosartorya sp., for example Neosartorya udagawae which can have, in some embodiments, the amino acid sequence of SEQ ID NO: 15, be a variant of the amino acid sequence of SEQ ID NO: 15 or be a fragment of the amino acid sequence of SEQ ID NO: 15. In an embodiment, the trehalase is from Aspergillus sp., for example Aspergillus flavus which can have, in some embodiments, the amino acid sequence of SEQ ID NO: 16, be a variant of the amino acid sequence of SEQ ID NO: 16 or be a fragment of the amino acid sequence of SEQ ID NO: 16. In an embodiment, the trehalase is from Fusarium sp., for example Fusarium oxysporum which can have, in some embodiments, the amino acid sequence of SEQ ID NO: 17, be a variant of the amino acid sequence of SEQ ID NO: 17 or be a fragment of the amino acid sequence of SEQ ID NO: 17. In an embodiment, the trehalase is from Escovopsis sp., for example Escovopsis weberi which can have, in some embodiments, the amino acid sequence of SEQ ID NO: 18, be a variant of the amino acid sequence of SEQ ID NO: 18 or be a fragment of the amino acid sequence of SEQ ID NO: 18. In an embodiment, the trehalase is from Microsporum sp., for example Microsporum gypseum which can have, in some embodiments, the amino acid sequence of SEQ ID NO: 19, be a variant of the amino acid sequence of SEQ ID NO: 19 or be a fragment of the amino acid sequence of SEQ ID NO: 19. In an embodiment, the trehalase is from Aspergillus sp., for example Aspergillus clavatus which can have, in some embodiments, the amino acid sequence of SEQ ID NO: 20, be a variant of the amino acid sequence of SEQ ID NO: 20 or be a fragment of the amino acid sequence of SEQ ID NO: 20. In an embodiment, the trehalase is from Metarhizium sp., for example Metarhizium anisopliae which can have, in some embodiments, the amino acid sequence of SEQ ID NO: 21, be a variant of the amino acid sequence of SEQ ID NO: 21 or be a fragment of the amino acid sequence of SEQ ID NO: 21. In an embodiment, the trehalase is from Ogataea sp., for example Ogataea parapolymorpha which can have, in some embodiments, the amino acid sequence of SEQ ID NO: 22, be a variant of the amino acid sequence of SEQ ID NO: 22 or be a fragment of the amino acid sequence of SEQ ID NO: 22. In an embodiment, the trehalase is from Kluyveromyces sp., for example Kluyveromyces marxianus which can have, in some embodiments, the amino acid sequence of SEQ ID NO: 23, be a variant of the amino acid sequence of SEQ ID NO: 23 or be a fragment of the amino acid sequence of SEQ ID NO: 23. In an embodiment, the trehalase is from Komagataella sp., for example Komagataella phaffii which can have, in some embodiments, the amino acid sequence of SEQ ID NO: 24, be a variant of the amino acid sequence of SEQ ID NO: 24 or be a fragment of the amino acid sequence of SEQ ID NO: 24. In an embodiment, the trehalase is from Ashbya sp., for example Ashbya gossypii which can have, in some embodiments, the amino acid sequence of SEQ ID NO: 25, be a variant of the amino acid sequence of SEQ ID NO: 25 or be a fragment of the amino acid sequence of SEQ ID NO: 25. In an embodiment, the trehalase is from Neurospora sp., for example Neurospora crassa which can have, in some embodiments, the amino acid sequence of SEQ ID NO: 26, be a variant of the amino acid sequence of SEQ ID NO: 7 or be a fragment of the amino acid sequence of SEQ ID NO: 7. In such embodiment, the trehalase can be encoded, for example, by the nucleic acid sequence of SEQ ID NO: 8, a variant of SEQ ID NO 8 or a fragment of SEQ ID NO: 8. In an embodiment, the trehalase is from Thielavia sp., for example Thielavia terrestris which can have, in some embodiments, the amino acid sequence of SEQ ID NO: 26, be a variant of the amino acid sequence of SEQ ID NO: 26 or be a fragment of the amino acid sequence of SEQ ID NO: 26. In an embodiment, the trehalase is from Aspergillus sp., for example Aspergillus lentulus which can have, in some embodiments, the amino acid sequence of SEQ ID NO: 27, be a variant of the amino acid sequence of SEQ ID NO: 27 or be a fragment of the amino acid sequence of SEQ ID NO: 27. In an embodiment, the trehalase is from Aspergillus sp., for example Aspergillus ochraceoroseus which can have, in some embodiments, the amino acid sequence of SEQ ID NO: 28, be a variant of the amino acid sequence of SEQ ID NO: 28 or be a fragment of the amino acid sequence of SEQ ID NO: 28. In an embodiment, the trehalase is from Rhizoctonia sp., for example Rhizoctonia solani which can have, in some embodiments, the amino acid sequence of SEQ ID NO: 29, be a variant of the amino acid sequence of SEQ ID NO: 29 or be a fragment of the amino acid sequence of SEQ ID NO: 29. In an embodiment, the trehalase is from Achlya sp., for example Achlya hypogyna which can have, in some embodiments, the amino acid sequence of SEQ ID NO: 30, be a variant of the amino acid sequence of SEQ ID NO: 30 or be a fragment of the amino acid sequence of SEQ ID NO: 30. In an embodiment, the trehalase is from Schizopora sp., for example Schizopora paradoxa which can have, in some embodiments, the amino acid sequence of SEQ ID NO: 31, be a variant of the amino acid sequence of SEQ ID NO: 31 or be a fragment of the amino acid sequence of SEQ ID NO: 31.

The glycoside hydrolase can use glycogen as a substrate. In an embodiment, the glycoside hydrolase can be a glycogen phosphorylase and/or a glycogen debranching enzyme. In yeasts, glycogen is degraded by Gph1p and Gdb1p enzymes, which are phosphorylase and debranching enzymes respectively. GPH1 progressively releases glucose-1-phosphate from linear alpha (1,4)-glucosidic bonds in glycogen but is not able to break alpha (1,4)-glucosidic bonds that are close to alpha (1,6)-branch linkages. The branches are resolved by GDP1, which eliminates branch points in a two-step process The degradation of glycogen and/or the presence of cleaved glycogen moieties (directly or indirectly from the generation of the inhibitory product), prior to fermentation, can be detrimental to the stability of the yeast as well as its performance of the recombinant yeast host cell during the fermentation. In such embodiments, the substrate/yeast cellular component can be glycogen or a glycogen-containing biological molecule.

This ability to express the first heterologous polypeptide can be conferred by introducing one or more first heterologous nucleic acid molecule in the recombinant yeast host cell. The first heterologous nucleic acid molecule include one or more heterologous promoter operatively associated with a sequence encoding the first heterologous polypeptide. In some embodiments, the yeast host cell, prior to the introduction of the first heterologous nucleic acid molecule encoding the first heterologous polypeptide, lacks the ability to exhibit hydrolase activity and, in some embodiments, to convert the unfermentable carbohydrate source into the fermentable carbohydrate source. In additional embodiments, the yeast host cell, prior to the introduction of the first heterologous nucleic acid molecule encoding the first heterologous polypeptide, has some (limited) ability to exhibit hydrolase activity and, in some embodiments, to convert the unfermentable carbohydrate source into the fermentable carbohydrate source.

The recombinant yeast host cell can include one or more copies of the first heterologous nucleic acid molecule. Alternatively, more than one type of first heterologous polypeptides can be expressed in the recombinant yeast host cell. In such embodiments, the recombinant yeast host cell can include one or more copies of different first heterologous nucleic acid molecules encoding different first heterologous polypeptides.

The expression of the first heterologous polypeptide is controlled, at least in part, by a first heterologous promoter (or a combination of first heterologous promoters). The first heterologous promoter is an inducible promoter and cannot be a constitutive promoter. The first heterologous promoter is capable of limiting the expression of the first heterologous polypeptide during the propagation phase of the recombinant yeast host cell. In some embodiments, the first heterologous promoter is capable of limiting the expression of the first heterologous polypeptide in a propagation which is performed under aerobic conditions (e.g., an aerobic or aerated propagation). The first heterologous promoter is capable of favoring the expression of the first heterologous polypeptide during the fermentation phase of the recombinant yeast host cell (e.g., a fermentation specific promoter). In some embodiments, the first heterologous promoter is capable of favoring the expression of the first heterologous polypeptide during a fermentation which is performed under anaerobic conditions (e.g., anaerobic fermentation). Even though the first heterologous promoter may allow some (limited) expression of the first heterologous polypeptide during the propagation phase of the recombinant yeast host cell, the first heterologous promoter favors (and in some embodiments only allows) the expression of the first heterologous polypeptide during the fermentation phase of the recombinant yeast host cell. It is important that the first heterologous promoter limits or prevents the expression/accumulation of the first heterologous polypeptide during the propagation phase of the recombinant yeast host cell so as to provide stability/improved fermentation performances to the propagated recombinant yeast host cell or to compositions comprising same.

The recombinant yeast host cell of the present disclosure is intended to be used in a commercial process for making a fermentation product. In such commercial process, the recombinant yeast host cell is first submitted to propagation (in a propagation medium) and then to fermentation (in a fermentation medium which differs from the propagation medium). As used in the context of the present disclosure, the expression “propagation” or “propagation phase” refers to an expansion phase of the commercial process in which the recombinant yeast host cells are propagated under aerobic conditions to maximize the conversion of a propagation medium into a propagated yeast biomass. As used in the context of the present disclosure, the expression “fermentation” or “fermentation phase” refers to a production phase of the commercial process in which the propagated yeast biomass is used to maximize the production of one or more desired fermentation products (usually under anaerobic conditions) from a fermentation medium (usually comprising fermentable carbohydrates). In some embodiments, the propagated recombinant yeast host cell can be used directly in a fermentation. In other embodiments, the propagated recombinant yeast host cell can be stored (e.g., placed in a storage phase) in a storage medium prior to the fermentation. In some embodiments, the storage medium comprises a source of unfermentable carbohydrates which is absent from the propagation medium (e.g., trehalose for example).

The first heterologous promoter (or combination thereof) can include without limitation anaerobic-regulated promoters (also referred to anaerobic specific promoters), heat shock-regulated promoters, oxidative stress response promoters and osmotic stress response promoters. As used in the context of the present disclosure, an anaerobic-regulated promoter refers to a promoter capable of favoring the expression of its associated open-reading frame (e.g., the nucleic acid molecule encoding the first heterologous polypeptide) in the presence of anaerobia (partial or complete). Anaerobic-regulated promoters include, but are not limited to, the promoter of the YER011W or tir1 gene (referred to as tir1p and which can have the nucleic acid sequence of SEQ ID NO: 10, a variant thereof or a fragment thereof), of the YFL020C or pau5 gene (referred to as pau5p and which can have the nucleic acid sequence of SEQ ID NO: 11, a variant thereof or a fragment thereof), of the YJR150C or dan1 gene (referred to as dan1p which can have the nucleic acid sequence of SEQ ID NO: 12, a variant thereof or a fragment thereof), of the YJL052W or tdh1 gene (referred to as tdh1p and which can have the nucleic acid sequence of SEQ ID NO: 39, a variant thereof or a fragment thereof), of the YER150W of the spi1 gene (referred to as spi1p and which can have the nucleic acid sequence of SEQ ID NO: 40, a variant thereof or a fragment thereof), of the YFR053C or the hxk1 gene (referred to as hxk1p and which can have the nucleic acid sequence of SEQ ID NO: 41, a variant thereof or a fragment thereof), of the YJR047C or the anb1 gene (referred to as anb1p which can have the nucleic acid sequence of SEQ ID NO: 42, a variant thereof or a fragment thereof), of the YDR343C or the hxt6 gene (referred to as hxt6p or phxt6 and which can have the nucleic acid sequence of SEQ ID NO: 43, a variant thereof or a fragment thereof), of the YLR043C or the trx1 gene (referred to as trx1p or ptrx1 and which can have the nucleic acid sequence of SEQ ID NO: 44, a variant thereof or a fragment thereof) and of the YBR085W or of the aac3 gene (referred to as aac3p and which can have the amino acid sequence of SEQ ID NO: 45, a variant thereof or a fragment thereof). In an embodiment, the anaerobic-regulated promoters comprises the promoter of the YER011W or tir1 gene (referred to as tir1p and which can have the nucleic acid sequence of SEQ ID NO: 10, a variant thereof or a fragment thereof), alone or in combination with other fermentation specific promoters. In an embodiment, the anaerobic-regulated promoters comprises the promoter of the YFL020C or pau5 gene (referred to as pau5p and which can have the nucleic acid sequence of SEQ ID NO: 11, a variant thereof or a fragment thereof), alone or in combination with another fermentation-specific promoter. In an embodiment, the anaerobic-regulated promoters comprises the promoter of the YJR150C or dan1 gene (referred to as dan1p which can have the nucleic acid sequence of SEQ ID NO: 12, a variant thereof or a fragment thereof), alone or in combination with other fermentation-specific promoters. In an embodiment, the anaerobic-regulated promoters comprises the promoter of the YJL052W or tdh1 gene (referred to as tdh1p and which can have the nucleic acid sequence of SEQ ID NO: 39, a variant thereof or a fragment thereof), alone or in combination with other fermentation specific promoters. In an embodiment, the anaerobic-regulated promoters comprises the promoter of the YER150W of the spi1 gene (referred to as spi1p and which can have the nucleic acid sequence of SEQ ID NO: 40, a variant thereof or a fragment thereof), alone or in combination with other fermentation specific promoters. In an embodiment, the anaerobic-regulated promoters comprises the promoter of the YFR053C or the hxk1 gene (referred to as hxk1p and which can have the nucleic acid sequence of SEQ ID NO: 41, a variant thereof or a fragment thereof), alone or in combination with other fermentation specific promoters. In an embodiment, the anaerobic-regulated promoters comprises the promoter of the YJR047C or the anb1 gene (referred to as anb1p which can have the nucleic acid sequence of SEQ ID NO: 42, a variant thereof or a fragment thereof), alone or in combination with other fermentation specific promoters. In an embodiment, the anaerobic-regulated promoters comprises the promoter of the YDR343C or the hxt6 gene (referred to as hxt6p or phxt6 and which can have the nucleic acid sequence of SEQ ID NO: 43, a variant thereof or a fragment thereof), alone or in combination with other fermentation specific promoters. In an embodiment, the anaerobic-regulated promoters comprises the promoter of the YLR043C or the trx1 gene (referred to as trx1p or ptrx1 and which can have the nucleic acid sequence of SEQ ID NO: 44, a variant thereof or a fragment thereof), a variant thereof or a fragment thereof), alone or in combination with other fermentation specific promoters. In an embodiment, the anaerobic-regulated promoters comprises the promoter of the aac3 gene (referred to as aac3p and which can have the amino acid sequence of SEQ ID NO: 45, a variant thereof or a fragment thereof), alone or in combination with other fermentation specific promoters.

In some embodiments, the first heterologous polypeptide can be intended to exert its biological activity mainly outside the recombinant yeast host cell, the first heterologous polypeptide can be selected based on its ability to be translocated outside the cell or alternatively modified to be secreted or remain associated with the external surface of the recombinant yeast host cell membrane. Some first heterologous polypeptide possess a signal sequence and are presumed to be secreted from the recombinant yeast host cell. For these first heterologous polypeptides, it is contemplated to use their native signal sequence or replace it with another signal sequence which will facilitate their secretion from the recombinant yeast host cell. For the other first heterologous polypeptides lacking a native signal sequence, it is possible to include an appropriate signal sequence allowing their secretion outside the cell, for example from by including a signal sequence from another first heterologous polypeptide or a signal sequence being recognized as such by the recombinant yeast host cell. In embodiments in which the hydrolase is intended to be secreted, it is expected to exert its enzymatic activity at least in part outside the recombinant yeast host cell on a substrate which may no be a yeast cellular component (because not physically associated with the recombinant yeast host cell).

In some embodiments, the secreted first heterologous polypeptides are released (e.g., secreted) in the fermentation medium and do not remain physically attached to the recombinant yeast cell. In alternative embodiments, the first heterologous polypeptides of the present disclosure can be secreted, but they remain physically associated with the recombinant yeast host cell. In an embodiment, at least one portion (usually at least one terminus) of the first heterologous polypeptide is bound, covalently, non-covalently and/or electrostatically for example, to cell wall (and in some embodiments to the cytoplasmic membrane). For example, the first heterologous polypeptide can be modified to bear one or more transmembrane domains, to have one or more lipid modifications (myristoylation, palmitoylation, farnesylation and/or prenylation), to interact with one or more membrane-associated polypeptide and/or to interactions with the cellular lipid rafts. While the first heterologous polypeptide may not be directly bound to the cell membrane or cell wall (e.g., such as when binding occurs via a tethering moiety), the polypeptide is nonetheless considered a “cell-associated” heterologous polypeptide according to the present disclosure.

In some embodiments, the first heterologous polypeptides can be expressed to be located at and associated to the cell wall of the recombinant yeast host cell. In some embodiments, the heterologous polypeptide is expressed to be located at and associated to the external surface of the cell wall of the host cell. Recombinant yeast host cells all have a cell wall (which includes a cytoplasmic membrane) defining the intracellular (e.g., internally-facing the nucleus) and extracellular (e.g., externally-facing) environments. The first heterologous polypeptide can be located at (and in some embodiments, physically associated to) the external face of the recombinant yeast host’s cell wall and, in further embodiments, to the external face of the recombinant yeast host’s cytoplasmic membrane. In the context of the present disclosure, the expression “associated to the external face of the cell wall/cytoplasmic membrane of the recombinant yeast host cell” refers to the ability of the first heterologous polypeptide to physically integrate (in a covalent or non-covalent fashion), at least in part, in the cell wall (and in some embodiments in the cytoplasmic membrane) of the recombinant yeast host cell. The physical integration can be attributed to the presence of, for example, a transmembrane domain on the heterologous polypeptide, a domain capable of interacting with a cytoplasmic membrane polypeptide on the heterologous polypeptide, a post-translational modification made to the heterologous polypeptide (e.g., lipidation), etc.

In some circumstances, it may be warranted to increase or provide cell association to some first heterologous polypeptides because they exhibit insufficient intrinsic cell association or simply lack intrinsic cell association. In such embodiment, it is possible to provide the first heterologous polypeptide as a chimeric construct by combining it with a tethering amino acid moiety which will provide or increase attachment to the cell wall of the recombinant yeast host cell. In such embodiment, the chimeric heterologous polypeptide will be considered “tethered”. It is preferred that the amino acid tethering moiety of the chimeric polypeptide be neutral with respect to the biological activity of the first heterologous polypeptide, e.g., does not interfere with the biological activity (such as, for example, the enzymatic activity) of the first heterologous polypeptide. In some embodiments, the association of the amino acid tethering moiety with the heterologous polypeptide can increase the biological activity of the heterologous polypeptide (when compared to the non-tethered, “free” form).

In an embodiment, a tethering moiety can be used to be expressed with the first heterologous polypeptide to locate the heterologous polypeptide to the wall of the recombinant yeast host cell. Various tethering amino acid moieties are known art and can be used in the chimeric polypeptides of the present disclosure. The tethering moiety can be a transmembrane domain found on another polypeptide and allow the chimeric polypeptide to have a transmembrane domain. In such embodiment, the tethering moiety can be derived from the FLO1 polypeptide. In still another example, the amino acid tethering moiety can be modified post-translation to include a glycosylphosphatidylinositol (GPI) anchor and allow the chimeric polypeptide to have a GPI anchor. GPI anchors are glycolipids attached to the terminus of a polypeptide (and in some embodiments, to the carboxyl terminus of a polypeptide) which allows the anchoring of the polypeptide to the cytoplasmic membrane of the cell membrane. Tethering amino acid moieties capable of providing a GPI anchor include, but are not limited to those associated with/derived from a SED1 polypeptide, a TIR1 polypeptide, a CWP2 polypeptide, a CCW12 polypeptide, a SPI1 polypeptide, a PST1 polypeptide or a combination of a AGA1 and a AGA2 polypeptide. In an embodiment, the tethering moiety provides a GPI anchor and, in still a further embodiment, the tethering moiety is derived from the SPI1 polypeptide or the CCW12 polypeptide.

The tethering amino acid moiety can be a variant of a known/native tethering amino acid moiety. The tethering amino acid moiety can be a fragment of a known/native tethering amino acid moiety or fragment of a variant of a known/native tethering amino acid moiety.

In embodiments in which an amino acid tethering moiety is desirable, the heterologous polypeptide can be provided as a chimeric polypeptide expressed by the recombinant yeast host cell and having one of the following formulae (provided from the amino (NH₂) to the carboxyl (COOH) orientation) :

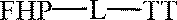

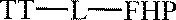

In both of these formulae, the residue “FHP” refers to the first heterologous polypeptide moiety, the residue “L” refers to the presence of an optional linker while the residue “TT” refers to an amino acid tethering moiety. In the chimeric polypeptides of formula (I), the amino terminus of the amino acid tether is located (directly or indirectly) at the carboxyl (COOH or C) terminus of the first heterologous polypeptide moiety. In the chimeric polypeptides of formula (II), the carboxy terminus of the amino acid tether is located (directly or indirectly) at the amino (NH₂ or N) terminus of the first heterologous polypeptide moiety. Embodiments of chimeric tethered heterologous polypeptides have been disclosed in WO2018/167670 and are included herein in their entirety.

In some embodiments, the first heterologous polypeptide can be intended to exert its biological activity mainly inside the recombinant yeast host cell, the first heterologous polypeptide can be selected based on its ability to be remain inside the cell or alternatively modified to remain inside the recombinant yeast host cell membrane. For example, the first heterologous polypeptide can be modified to remove its signal sequence to favor intracellular expression and maintenance.

### Second Heterologous Polypeptide

In some embodiments, the recombinant yeast host cell of the present disclosure has the ability to express a second heterologous polypeptide. The second heterologous polypeptide refers an enzyme (or to a combination of enzymes) having saccharolytic activity. The second heterologous polypeptide is different from the first heterologous polypeptide. This ability to express the second heterologous polypeptide can be conferred by introducing one or more second heterologous nucleic acid molecule in the recombinant yeast host cell. The second heterologous nucleic acid molecule encodes the second heterologous polypeptide. The recombinant yeast host cell can include one or more copies of the second heterologous nucleic acid molecule. Alternatively, more than one type of second heterologous polypeptides can be expressed in the recombinant yeast host cell. In such embodiments, the recombinant yeast host cell can include one or more copies of different second heterologous nucleic acid molecules encoding different second heterologous polypeptides.

The expression of coding sequence of the second heterologous nucleic acid molecule can be controlled, at least in part, by a second heterologous promoter or a combination of second heterologous promoters. The second heterologous promoter can be constitutive or inducible. The second heterologous promoter can allow the expression of the second heterologous polypeptide during the propagation phase and/or the fermentation phase of the recombinant yeast host cell. As such, in some embodiments, the second heterologous nucleic acid molecule can include one or more promoter operatively associated with a sequence coding for a saccharolytic enzyme.

As used in the context of the present disclosure, a “saccharolytic enzyme” can be any enzyme (or combination of enzymes) involved in carbohydrate digestion, metabolism and/or hydrolysis, including amylases, cellulases, hemicellulases, cellulolytic and amylolytic accessory enzymes, inulinases, levanases, and pentose sugar utilizing enzymes. One embodiment of the saccharolytic enzyme is an amylolytic enzyme. As used herein, the expression “amylolytic enzyme” refers to a class of enzymes capable of hydrolyzing starch or hydrolyzed starch. Amylolytic enzymes include, but are not limited to alpha-amylases (EC 3.2.1.1, sometimes referred to fungal alpha-amylase, see below), maltogenic amylase (EC 3.2.1.133), glucoamylase (EC 3.2.1.3), glucan 1,4-alpha-maltotetraohydrolase (EC 3.2.1.60), pullulanase (EC 3.2.1.41), iso-amylase (EC 3.2.1.68) and amylomaltase (EC 2.4.1.25). In an embodiment, the one or more amylolytic enzymes can be an alpha-amylase from Aspergillus oryzae, a maltogenic alpha-amylase from Geobacillus stearothermophilus, a glucoamylase (GA) from Saccharomycopsis fibuligera, a glucan 1,4-alpha-maltotetraohydrolase from Pseudomonas saccharophila, a pullulanase from Bacillus naganoensis, a pullulanase from Bacillus acidopullulyticus, an iso-amylase from Pseudomonas amyloderamosa, and/or amylomaltase from Thermus thermophilus. Some amylolytic enzymes have been described in WO2018/167670 and are incorporated herein by reference.

In specific embodiments, the recombinant yeast host cell the second heterologous polypeptide can comprise a heterologous glucoamylase. Many microbes produce an amylase to degrade extracellular starches. In addition to cleaving the last α(1- 4) glycosidic linkages at the non-reducing end of amylose and amylopectin, yielding glucose, γ-amylase will cleave α(1-6) glycosidic linkages. The heterologous glucoamylase can be derived from any organism. In an embodiment, the heterologous polypeptide is derived from a γ-amylase, such as, for example, the glucoamylase of Saccharomycoces filbuligera (e.g., encoded by the glu 0111 gene). Examples of recombinant yeast host cells expressing a heterologous glucoamylase are described in WO 2011/153516 as well as in WO 2017/037614 and herewith incorporated in its entirety. In an embodiment, the glucoamlyase has the amino acid sequence of SEQ ID NO: 1, 32 or 34, a variant of the amino acid sequence of SEQ ID NO: 1, 32 or 34 having glucoamylase activity or a fragment of the amino acid sequence of SEQ ID NO: 1, 32 or 34 having glucoamlyase activity.

### Third Heterologous Polypeptide

In some embodiments, the recombinant yeast host cell of the present disclosure has the ability to express a third heterologous polypeptide. The third heterologous polypeptide refers a polypeptide (or a combination of polypeptides) involved in modulating the production of formate. The activity of the third heterologous polypeptide can increase or decrease the production of formation. The third heterologous polypeptide can be involved in the production formate, the breakdown of formate or the regulation of the production/breakdown of formate. This ability to express the third heterologous polypeptide can be conferred by introducing one or more third heterologous nucleic acid molecule in the recombinant yeast host cell. The third heterologous nucleic acid molecule encodes the third heterologous polypeptide. The recombinant yeast host cell can include one or more copies of the third heterologous nucleic acid molecule. Alternatively, more than one type of third heterologous polypeptides can be expressed in the recombinant yeast host cell. In such embodiments, the recombinant yeast host cell can include one or more copies of different third heterologous nucleic acid molecules encoding different third heterologous polypeptides.

The expression of coding sequence of the third heterologous nucleic acid molecule can be controlled, at least in part, by a third heterologous promoter or a combination of third heterologous promoters. The third heterologous promoter can be constitutive or inducible. The third heterologous promoter can allow the expression of the third heterologous polypeptide during the propagation phase and/or the fermentation phase of the recombinant yeast host cell. As such, in some embodiments, the third heterologous nucleic acid molecule can include one or more promoter operatively associated with a sequence coding for a polypeptide involved in modulating the production of formate.

In some specific embodiments, the third heterologous polypeptide comprises a heterologous enzyme that function to anabolize (form) formate. In some embodiments, the heterologous enzyme that function to anabolize formate is a heterologous pyruvate formate lyase (PFL). Heterologous PFL of the present disclosure include, but are not limited to, the PFLA polypeptide, a polypeptide encoded by a pfla gene ortholog or paralog, the PFLB polyeptide or a polypeptide encoded by a pflb gene ortholog or paralog.

In an embodiment, the third heterologous polypeptide comprises PFLA. In some embodiments, PFLA can have the amino acid sequence of SEQ ID NO: 3, be a variant of the amino acid sequence of SEQ ID NO: 3 having pyruvate formate lyase activity or a fragment of the amino acid sequence of SEQ ID NO: 3 having pyruvate formate lyase activity. In another embodiment, the third heterologous polypeptide comprises PFLB. In some embodiments, PFLB can have the amino acid sequence of SEQ ID NO: 4, be a variant of the amino acid sequence of SEQ ID NO: 4 having pyruvate formate lyase activity or be a fragment of the amino acid sequence of SEQ ID NO: 4 having pyruvate formate lyase activity. In yet another embodiment, the third heterologous polypeptide comprises PFLA and PFLB.

In an embodiment, the recombinant yeast host cell of the present disclosure can have native formate dehydrogenase (FDH) gene(s) (such as, for example, FDH1 and FDH2) and are capable of expressing the native FDH gene(s). In another embodiment, the recombinant yeast host cell of the present disclosure can be selected or modified to have inactivated native FDH gene(s) (such as, for example, FDH1 and FDH2) and have a limited or no ability in expressing native FDH gene(s).

In some specific embodiments, the third heterologous polypeptide comprises a heterologous enzyme that function to catabolize (breakdown) formate, such as, for example, formate dehydrogenases (FDH). Heterologous FDH of the present disclosure include, but are not limited to, the FDH1 polypeptide, a polypeptide encoded by a fdh1 gene ortholog or paralog, the FDH2 polypeptide or a polypeptide encoded by a fdh2 gene ortholog or paralog.

In an embodiment, the third heterologous polypeptide comprises FDH1. In some embodiments, FDH1 can have the amino acid sequence of SEQ ID NO: 5, be a variant of the amino acid sequence of SEQ ID NO: 5 having formate dehydrogenase activity or a fragment of the amino acid sequence of SEQ ID NO: 5 having formate dehydrogenase activity.

### Fourth Heterologous Polypeptide

In some embodiments, the recombinant yeast host cell of the present disclosure has the ability to express a fourth heterologous polypeptide. The fourth heterologous polypeptide is a polypeptide (or a combination of polypeptides) involved converting acetyl-CoA into an alcohol, such as ethanol. This ability to express the fourth heterologous polypeptide can be conferred by introducing one or more fourth heterologous nucleic acid molecule in the recombinant yeast host cell. The fourth heterologous nucleic acid molecule encodes the fourth heterologous polypeptide. The recombinant yeast host cell can include one or more copies of the fourth heterologous nucleic acid molecule. Alternatively, more than one type of fourth heterologous polypeptides can be expressed in the recombinant yeast host cell. In such embodiments, the recombinant yeast host cell can include one or more copies of different fourth heterologous nucleic acid molecules encoding different fourth heterologous polypeptides.

The expression of the coding sequence of the fourth heterologous nucleic acid molecule can be controlled, at least in part, by a fourth heterologous promoter or a combination of fourth heterologous promoters. The fourth heterologous promoter can be constitutive or inducible. The fourth heterologous promoter can allow the expression of the fourth heterologous polypeptide during the propagation phase and/or the fermentation phase of the recombinant yeast host cell. As such, in some embodiments, the fourth heterologous nucleic acid molecule can include one or more promoter operatively associated with a sequence coding for a polypeptide involved in converting acetyl-CoA into an alcohol (such as ethanol).

The fourth heterologous polypeptides can comprise a polypeptide having acetaldehyde dehydrogenase activity, alcohol dehydrogenase activity or both. In a heterologous acetaldehyde dehydrogenases (AADH), a heterologous alcohol dehydrogenases (ADH), and/or and heterologous bifunctional acetaldehyde/alcohol dehydrogenases (ADHE) such as those described in U.S. Pat. Serial No. 8,956,851 and WO 2015/023989. Heterologous AADHs of the present disclosure include, but are not limited to, the ADHE polypeptides or a polypeptide encoded by an adhe gene ortholog or paralog. In an embodiment, the ADHE can comprise an amino acid sequence of SEQ ID NO: 2, a variant of the amino acid sequence of SEQ ID NO: 2 or a fragment of the amino acid sequence of SEQ ID NO: 2.

### Fifth Heterologous Polypeptide

In some embodiments, the recombinant yeast host cell of the present disclosure has the ability to express a fifth heterologous polypeptide. The fifth heterologous polypeptide refers a polypeptide (or a combination of polypeptides) involved in modulating the production of glycerol. The fifth heterologous polypeptide can increase or decrease the production of glycerol. The fifth heterologous polypeptide can be involved in the production glycerol, the breakdown of glycerol, the transport of glycerol or the regulation of the production/breakdown of glycerol. This ability to express the fifth heterologous polypeptide can be conferred by introducing one or more fifth heterologous nucleic acid molecule in the recombinant yeast host cell. The fifth heterologous nucleic acid molecule encodes the fifth heterologous polypeptide. The recombinant yeast host cell can include one or more copies of the fifth heterologous nucleic acid molecule. Alternatively, more than one type of fifth heterologous polypeptides can be expressed in the recombinant yeast host cell. In such embodiments, the recombinant yeast host cell can include one or more copies of different fifth heterologous nucleic acid molecules encoding different fifth heterologous polypeptides.

The expression of the coding sequence the fifth heterologous nucleic acid molecule can be controlled, at least in part, by a fifth heterologous promoter or a combination of fifth heterologous promoters. The fifth heterologous promoter can be constitutive or inducible. The fifth heterologous promoter can allow the expression of the fifth heterologous polypeptide during the propagation phase and/or the fermentation phase of the recombinant yeast host cell. As such, in some embodiments, the fifth heterologous nucleic acid molecule can include one or more promoter operatively associated with a sequence coding for a polypeptide involved in modulating the production of glycerol.

In some embodiments, the recombinant yeast host cell can bear or be selected to bear one or more genetic modifications to reduce, and in an embodiment, inhibit one or more native enzymes that function to produce glycerol. As used in the context of the present disclosure, the expression “reducing the production of one or more native enzymes that function to produce glycerol” refers to a genetic modification which limits or impedes the expression of genes associated with one or more native polypeptides (in some embodiments enzymes) that function to produce glycerol, when compared to a corresponding yeast strain which does not bear such genetic modification. In some instances, the additional genetic modification reduces but still allows the production of one or more native polypeptides that function to produce glycerol. In other instances, the genetic modification inhibits the production of one or more native enzymes that function to produce glycerol. Polypeptides that function to produce glycerol refer to polypeptides which are endogenously found in the recombinant yeast host cell. Native enzymes that function to produce glycerol include, but are not limited to, the GPD1 and the GPD2 polypeptide (also referred to as GPD1 and GPD2 respectively) as well as the GPP1 and the GPP2 polypeptides (also referred to as GPP1 and GPP2 respectively). In an embodiment, the recombinant yeast host cell bears a genetic modification in at least one of the gpd1 gene (encoding the GPD1 polypeptide), the gpd2 gene (encoding the GPD2 polypeptide), the gpp1 gene (encoding the GPP1 polypeptide) or the gpp2 gene (encoding the GPP2 polypeptide). In another embodiment, the recombinant yeast host cell bears a genetic modification in at least two of the gpd1 gene (encoding the GPD1 polypeptide), the gpd2 gene (encoding the GPD2 polypeptide), the gpp1 gene (encoding the GPP1 polypeptide) or the gpp2 gene (encoding the GPP2 polypeptide). Examples of recombinant yeast host cells bearing such genetic modification(s) leading to the reduction in the production of one or more native enzymes that function to produce glycerol are described in WO 2012/138942. In some embodiments, the recombinant yeast host cell has a genetic modification (such as a genetic deletion or insertion) only in one enzyme that functions to produce glycerol, in the gpd2 gene, which would cause the host cell to have a knocked-out gpd2 gene. In some embodiments, the recombinant yeast host cell can have a genetic modification in the gpd1 gene and the gpd2 gene resulting is a recombinant yeast host cell being knock-out for the gpd1 gene and the gpd2 gene. In some specific embodiments, the recombinant yeast host cell can have be a knock-out for the gpd1 gene and have duplicate copies of the gpd2 gene (in some embodiments, under the control of the gpd1 promoter). In still another embodiment (in combination or alternative to the genetic modification described above). In yet another embodiment, the recombinant yeast host cell includes its native genes coding for the GPP/GDP polypeptide(s).

The fifth heterologous polypeptide can comprise polypeptides facilitating the transport of glycerol in the recombinant yeast host cell. For example, the fifth heterologous polypeptide is able to transport glycerol. Native enzymes that function to transport glycerol synthesis include, but are not limited to, the FPS1 polypeptide as well as the STL1 polypeptide. The FPS1 polypeptide is a glycerol exporter and the STL1 polypeptide functions to import glycerol in the recombinant yeast host cell. By either reducing or inhibiting the expression of the FPS1 polypeptide and/or increasing the expression of the STL1 polypeptide, it is possible to control, to some extent, glycerol transport.

In an embodiment, the fifth heterologous polypeptide comprises STL1. The STL1 polypeptide is natively expressed in yeasts and fungi, therefore the heterologous polypeptide functioning to import glycerol can be derived from yeasts and fungi. STL1 genes encoding the STL1 polypeptide include, but are not limited to, Saccharomyces cerevisiae Gene ID: 852149, Candida albicans, Kluyveromyces lactis Gene ID: 2896463, Ashbya gossypii Gene ID: 4620396, Eremothecium sinecaudum Gene ID: 28724161, Torulaspora delbrueckii Gene ID: 11505245, Lachancea thermotolerans Gene ID: 8290820, Phialophora attae Gene ID: 28742143, Penicillium digitatum Gene ID: 26229435, Aspergillus oryzae Gene ID: 5997623, Aspergillus fumigatus Gene ID: 3504696, Talaromyces atroroseus Gene ID: 31007540, Rasamsonia emersonii Gene ID: 25315795, Aspergillus flavus Gene ID: 7910112, Aspergillus terreus Gene ID: 4322759, Penicillium chrysogenum Gene ID: 8310605, Alternaria alternata Gene ID : 29120952, Paraphaeosphaeria sporulosa Gene ID: 28767590, Pyrenophora tritici-repentis Gene ID: 6350281, Metarhizium robertsii Gene ID: 19259252, Isaria fumosorosea Gene ID: 30023973, Cordyceps militaris Gene ID: 18171218, Pochonia chlamydosporia Gene ID: 28856912, Metarhizium majus Gene ID: 26274087, Neofusicoccum parvum Gene ID:19029314, Diplodia corticola Gene ID: 31017281, Verticillium dahliae Gene ID: 20711921, Colletotrichum gloeosporioides Gene ID: 18740172, Verticillium albo-atrum Gene ID: 9537052, Paracoccidioides lutzii Gene ID: 9094964, Trichophyton rubrum Gene ID: 10373998, Nannizzia gypsea Gene ID: 10032882, Trichophyton verrucosum Gene ID: 9577427, Arthroderma benhamiae Gene ID: 9523991, Magnaporthe oryzae Gene ID: 2678012, Gaeumannomyces graminis var. tritici Gene ID: 20349750, Togninia minima Gene ID: 19329524, Eutypa lata Gene ID: 19232829, Scedosporium apiospermum Gene ID: 27721841, Aureobasidium namibiae Gene ID: 25414329, Sphaerulina musiva Gene ID: 27905328 as well as Pachysolen tannophilus GenBank Accession Numbers JQ481633 and JQ481634, Saccharomyces paradoxus STL1 and Pichia sorbitophilia. In an embodiment, the STL1 polypeptide is encoded by Saccharomyces cerevisiae Gene ID: 852149. In an embodiment, the STL1 polypeptide has the amino acid sequence of SEQ ID NO: 6, is a variant of the amino acid sequence of SEQ ID NO: 6 or is a fragment of the amino acid sequence of SEQ ID NO: 6.

### Sixth Heterologous Polypeptide

In some embodiments, the recombinant yeast host cell of the present disclosure has the ability to express a sixth heterologous polypeptide. The sixth heterologous polypeptide refers a polypeptide (or a combination of polypeptides) involved in the production of trehalose. The sixth heterologous polypeptide can be involved in the production trehalose, the transport of trehalose or the regulation of the production of trehalose. This ability to express the sixth heterologous polypeptide can be conferred by introducing one or more sixth heterologous nucleic acid molecule in the recombinant yeast host cell. The sixth heterologous nucleic acid molecule encodes the sixth heterologous polypeptide. The recombinant yeast host cell can include one or more copies of the sixth heterologous nucleic acid molecule. Alternatively, more than one type of sixth heterologous polypeptides can be expressed in the recombinant yeast host cell. In such embodiments, the recombinant yeast host cell can include one or more copies of different sixth heterologous nucleic acid molecules encoding different sixth heterologous polypeptides.

The expression of the coding sequence of the sixth heterologous nucleic acid molecule can be controlled, at least in part, by a sixth heterologous promoter or a combination of sixth heterologous promoters. The sixth heterologous promoter can be constitutive or inducible. The sixth heterologous promoter can allow the expression of the sixth heterologous polypeptide during the propagation phase and/or the fermentation phase of the recombinant yeast host cell. As such, in some embodiments, the sixth heterologous nucleic acid molecule can include one or more promoter operatively associated with a sequence coding for a polypeptide involved in modulating the production of trehalose.

The sixth heterologous polypeptide can include one or more enzymes involved in trehalose production such as, for example, TPS1, TPS2, HXH1, HXK2, GLK1, PGM1, PGM2 and UGP1 as well as orthologs and paralogs encoding these enzymes.

In an embodiment, the sixth heterologous polypeptide comprise a trehalose-6-phosphate (trehalose-6-P) synthase and/or a trehalose-6-phosphate phosphatase. As used herein, the term “trehalose-6-phosphate synthase” refers to an enzyme capable of catalyzing the conversion of glucose-6-phosphate and UDP-D-glucose to α-α-trehalose-6-phosphate and UDP. In Saccharomyces cerevisiae, the trehalose-6-phosphate synthase gene can be referred to TPS1 (SGD:S000000330, Gene ID: 852423), BYP1, CIF1, FDP1, GGS1, GLC6 or TSS1. The recombinant yeast host cell of the present disclosure can include a heterologous nucleic acid molecule coding for TPS1, a variant thereof, a fragment thereof or for a polypeptide encoded by a TPS1 gene ortholog or paralog. As also used herein, the term “trehalose-6-phosphate phosphatase” refers to an enzyme capable of catalyzing the conversion of α-α-trehalose-6-phosphate and H₂O into phosphate and trehalose. In Saccharomyces cerevisiae, the trehalose-6-phosphate phosphatase gene can be referred to TPS2 (SGD:S000002481, Gene ID: 851646), HOG2 or PFK3. The recombinant yeast host cell of the present disclosure can express a heterologous TPS2 (as well as a variant or a fragment thereof) from any origin including, but not limited to Saccharomyces cerevisiae (Gene ID: 851646), Arabidopsis thaliana (Gene ID: 838269), Schizosaccharomyces pombe (Gene ID: 2543109), Fusarium pseudograminearum (Gene ID: 20363081), Sugiyamaella lignohabitans (Gene ID: 30036691), Chlamydomonas reinhardtii (Gene ID: 5727896), Phaeodactylum tricornutum (Gene ID: 7194914), Candida albicans (Gene ID: 3636892), Kluyveromyces marxianus (Gene ID: 34714509), Scheffersomyces stipitis (Gene ID: 4840387), Spathaspora passalidarum (Gene ID: 18869689), Emiliania huxleyi (Gene ID: 17270873) or Pseudogymnoascus destructans (Gene ID: 36290309). The recombinant yeast host cell of the present disclosure can include a nucleic acid molecule coding for TPS2, a variant thereof, a fragment thereof or for a polypeptide encoded by a TPS2 gene ortholog or paralog.

The sixth heterologous polypeptide can include a polypeptide involved in regulating trehalose production. In Saccharomyces cerevisiae, polypeptides involved in regulating trehalose production include, but are not limited to TPS3 and TSL1. In some specific embodiment, the polypeptide involved in regulating trehalose production is TSL1. The recombinant yeast host cell of the present disclosure can express a heterologous TSL1 (as well as a variant or a fragment thereof) from any origin including, but not limited to Saccharomyces cerevisiae (SGD:S000004566, Gene ID 854872), Gallus gallus (Gene ID107050801), Kluyveromyces marxianus (Gene ID: 34714558), Saccharomyces eubayanus (Gene ID: 28933129), Schizosaccharomyces japonicus (Gene ID: 7049746), Pichia kudriavzevii (Gene ID: 31691677) or Hydra vulgaris (Gene ID 105848257). In a specific embodiments, the recombinant yeast host cell of the present disclosure includes a nucleic acid molecule encoding the amino acid sequence of SEQ ID NO: 13, a variant of the amino acid sequence of SEQ ID NO: 13 or a fragment of the amino acid sequence of SEQ ID NO: 13.

### Seventh Heterologous Polypeptide

In some embodiments, the recombinant yeast host cell of the present disclosure has the ability to express a seventh heterologous polypeptide. The seventh heterologous polypeptide refers a polypeptide (or a combination of polypeptides) having glyceraldehyde-3-phosphate dehydrogenase activity. This ability to express the seventh heterologous polypeptide can be conferred by introducing one or more seventh heterologous nucleic acid molecule in the recombinant yeast host cell. The seventh heterologous nucleic acid molecule encodes the seventh heterologous polypeptide. The recombinant yeast host cell can include one or more copies of the seventh heterologous nucleic acid molecule. Alternatively, more than one type of seventh heterologous polypeptides can be expressed in the recombinant yeast host cell. In such embodiments, the recombinant yeast host cell can include one or more copies of different seventh heterologous nucleic acid molecules encoding different seventh heterologous polypeptides.

The expression of the coding sequence of the seventh heterologous nucleic acid molecule can be controlled, at least in part, by a seventh heterologous promoter or a combination of seventh heterologous promoters. The seventh heterologous promoter can be constitutive or inducible. The seventh heterologous promoter can allow the expression of the seventh heterologous polypeptide during the propagation phase and/or the fermentation phase of the recombinant yeast host cell. As such, in some embodiments, the seventh heterologous nucleic acid molecule can include one or more promoter operatively associated with a sequence coding for a polypeptide having glyceraldehyde-3-phosphate dehydrogenase activity.

In one embodiment, the seventh heterologous polypeptide comprises a NADP⁺/NAD⁺ dependent glyceraldehyde-3-phosphate dehydrogenase (EC1.2.1.90) and allows the conversion of NADP⁺ to NADPH and/or NAD⁺ to NAD⁺. Enzymes of EC1.2.1.90 can use NADP⁺ or NAD⁺ as a cofactor. In some embodiments, glyceraldehyde-3-phosphate dehydrogenase uses NADP⁺ and/or NAD⁺ as a cofactor. In one embodiment, the glyceraldehyde-3-phosphate dehydrogenase is encoded by a GAPN gene. In one embodiment, the glyceraldehyde-3-phosphate dehydrogenase is GAPN. Examples of the GAPN polypeptides have been disclosed in PCT/IB2019/060527 filed on Dec. 6, 2019 and herewith incorporated in its entirety.

In some embodiments, the glyceraldehyde-3-phosphate dehydrogenase can be derived from a bacteria, for example, from the genus Streptococcus and, in some instances, from the species Strepotococcus mutans. In some embodiments, the glyceraldehyde-3-phosphate dehydrogenase can be derived from a bacteria, for example, from the genus Lactobacillus and, in some instances, from the species Lactobacillus delbrueckii. In some embodiments, the glyceraldehyde-3-phosphate dehydrogenase can be derived from a bacteria, for example, from the genus Streptococcus and, in some instances, from the species Strepotococcus thermophilus. In some embodiments, the glyceraldehyde-3-phosphate dehydrogenase can be derived from a bacteria, for example, from the genus Streptococcus and, in some instances, from the species Strepotococcus macacae. In some embodiments, the glyceraldehyde-3-phosphate dehydrogenase can be derived from a bacteria, for example, from the genus Streptococcus and, in some instances, from the species Strepotococcus hyointestinalis. In some embodiments, the glyceraldehyde-3-phosphate dehydrogenase can be derived from a bacteria, for example, from the genus Streptococcus and, in some instances, from the species Strepotococcus urinalis. In some embodiments, the glyceraldehyde-3-phosphate dehydrogenase can be derived from a bacteria, for example, from the genus Streptococcus and, in some instances, from the species Strepotococcus canis. In some embodiments, the glyceraldehyde-3-phosphate dehydrogenase can be derived from a bacteria, for example, from the genus Streptococcus and, in some instances, from the species Strepotococcus thoraltensis. In some embodiments, the glyceraldehyde-3-phosphate dehydrogenase can be derived from a bacteria, for example, from the genus Streptococcus and, in some instances, from the species Strepotococcus dysgalactiae. In some embodiments, the glyceraldehyde-3-phosphate dehydrogenase can be derived from a bacteria, for example, from the genus Streptococcus and, in some instances, from the species Strepotococcus pyogenes. In some embodiments, the glyceraldehyde-3-phosphate dehydrogenase can be derived from a bacteria, for example, from the genus Streptococcus and, in some instances, from the species Strepotococcus ictaluri. In some embodiments, the glyceraldehyde-3-phosphate dehydrogenase can be derived from a bacteria, for example, from the genus Clostridium and, in some instances, from the species Clostridium perfringens. In some embodiments, the glyceraldehyde-3-phosphate dehydrogenase can be derived from a bacteria, for example, from the genus Clostridium and, in some instances, from the species Clostridium chromiireducens. In some embodiments, the glyceraldehyde-3-phosphate dehydrogenase can be derived from a bacteria, for example, from the genus Clostridium and, in some instances, from the species Clostridium botulinum. In some embodiments, the glyceraldehyde-3-phosphate dehydrogenase can be derived from a bacteria, for example, from the genus Bacillus and, in some instances, from the species Bacillus cereus. In some embodiments, the glyceraldehyde-3-phosphate dehydrogenase can be derived from a bacteria, for example, from the genus Bacillus and, in some instances, from the species Bacillus anthracis. In some embodiments, the glyceraldehyde-3-phosphate dehydrogenase can be derived from a bacteria, for example, from the genus Bacillus and, in some instances, from the species Bacillus thuringiensis. In some embodiments, the glyceraldehyde-3-phosphate dehydrogenase can be derived from a bacteria, for example, from the genus Pyrococcus and, in some instances, from the species Pyrococcus furiosus.

Embodiments of glyceraldehyde-3-phosphate dehydrogenase can also be derived, without limitation, from the following (the number in brackets correspond to the Gene ID number): Triticum aestivum (543435); Streptococcus mutans (1028095); Streptococcus agalactiae (1013627); Streptococcus pyogenes (901445); Clostridioides difficile (4913365); Mycoplasma mycoides subsp. mycoides SC str. (2744894); Streptococcus pneumoniae (933338); Streptococcus sanguinis (4807521); Acinetobacter pittii (11638070); Clostridium botulinum A str. (5185508); [Bacillus thuringiensis] serovar konkukian str. (2857794); Bacillus anthracis str. Ames (1088724); Phaeodactylum tricornutum (7199937); Emiliania huxleyi (17251102); Zea mays (542583); Helianthus annuus (110928814); Streptomyces coelicolor (1101118); Burkholderia pseudomallei (3097058, 3095849); variants thereof as well as fragments thereof.

Additional embodiments of glyceraldehyde-3-phosphate dehydrogenase can also be derived, without limitation, from the following (the number in brackets correspond to the Pubmed Accession number): Streptococcus macacae (WP_003081126.1), Streptococcus hyointestinalis (WP_115269374.1), Streptococcus urinalis (WP_006739074.1), Streptococcus canis ( WP_003044111.1), Streptococcus pluranimalium (WP_104967491.1), Streptococcus equi (WP_012678132.1), Streptococcus thoraltensis (WP_018380938.1), Streptococcus dysgalactiae (WP_138125971.1), Streptococcus halotolerans (WP_062707672.1), Streptococcus pyogenes (WP_136058687.1), Streptococcus ictaluri (WP_008090774.1), Clostridium perfringens (WP_142691612.1), Clostridium chromiireducens (WP_079442081.1), Clostridium botulinum (WP_012422907.1), Bacillus cereus (WP_000213623.1), Bacillus anthracis (WP_098340670.1), Bacillus thuringiensis (WP_087951472.1), Pyrococcus furiosus (WP_011013013.1) as well as variants thereof and fragments thereof.

In an embodiment, the recombinant yeast host cell comprises the second heterologous nucleic acid molecule and is capable of expressing the second heterologous polypeptide only or optionally in combination with any one of the third, the fourth, the fifth, the sixth or the seventh heterologous nucleic acid molecule. In an embodiment, the recombinant yeast host cell comprises the third heterologous nucleic acid molecule and is capable of expressing the third heterologous polypeptide only or optionally in combination with any one of the second, the fourth, the fifth, the sixth or the seventh heterologous nucleic acid molecule. In an embodiment, the recombinant yeast host cell comprises the fourth heterologous nucleic acid molecule and is capable of expressing the fourth heterologous polypeptide only or optionally in combination with any one of the second, the third, the fifth, the sixth or the seventh heterologous nucleic acid molecule. In an embodiment, the recombinant yeast host cell comprises the fifth heterologous nucleic acid molecule and is capable of expressing the fifth heterologous polypeptide only or optionally in combination with any one of the second, the third, the fourth, the sixth or the seventh heterologous nucleic acid molecule. In an embodiment, the recombinant yeast host cell comprises the sixth heterologous nucleic acid molecule and is capable of expressing the second heterologous polypeptide only or optionally in combination with any one of the second, the third, the fourth, the fifth, or the seventh heterologous nucleic acid molecule. In an embodiment, the recombinant yeast host cell comprises the seventh heterologous nucleic acid molecule and is capable of expressing the second heterologous polypeptide only or optionally in combination with any one of the second, the third, the fourth, the fifth or the sixth heterologous nucleic acid molecule.

### Process for Making a Population of Propagated Recombinant Yeast Host Cell

The present disclosure provides, in embodiments, a process for making a population of propagated recombinant yeast host cells exhibiting increased stability during storage and/or improve fermentation performance. The process comprises contacting the recombinant yeast host cell described herein with a propagation medium, under conditions so as to allow or favor the propagation of the recombinant yeast host cell. The propagation process can be a continuous method, a batch method or a fed-batch method. The propagation medium can comprise a carbon source (such as, for example, molasses, sucrose, glucose, dextrose syrup, ethanol, corn, glycerol, corn steep liquor and/or a lignocellulosic biomass), a nitrogen source (such as, for example, ammonia or another inorganic source of nitrogen) and a phosphorous source (such as, for example, phosphoric acid or another inorganic source of phosphorous). In some embodiments, the propagation medium does not include an unfermentable carbohydrate source which can be hydrolyzed by the first heterologous polypeptide. The propagation medium can further comprises additional micronutrients such as vitamins and/or minerals to support the propagation of the recombinant yeast host cell.

In the propagation process, the recombinant yeast host cell is placed in a propagation medium which can, in some embodiments, allow for a specific growth rate of 0.25, 0.24, 0.23, 0.22, 0.21, 0.20, 0.19, 0.18, 0.17, 0.16 or 0.15 h⁻¹ or less. In order to limit the growth rate of the recombinant yeast host cell, in some embodiments, the process can further comprise controlling the addition of nutriments, such as carbohydrates. Limiting the growth rate of the recombinant yeast host cell during propagation can be achieved by maintaining the concentration of carbohydrates below 0.1, 0.01, 0.001 or 0.0001 weight % with respect to the volume of the culture medium. Controlling the concentration of the carbohydrates of the propagation medium can be done by various means known in the art and can involve sampling the culture medium at various intervals, determining the carbohydrate concertation, alcohol concentration and/or gas (CO₂) concentration in those samples and adding or refraining from adding, if necessary additional carbohydrates in the culture medium to accelerate or decelerate the growth of the recombinant yeast host cell. In some embodiments, the process provides for adding nitrogen and/or phosphorous to match/support the growth rate of the recombinant yeast host cell.

The propagation process can be conducted under high aeration conditions. For example, in some embodiments, the process can include controlling the aeration of the vessel to achieve a specific aeration rate, for example, of at least 0.2, 0.3, 0.4, 0.5, 0.6, 0.7, 0.8, 0.9, 1.0, 1.1, 1.2 or 1.3 air volume/vessel volume/minute.

The propagation process can be conducted at a specific pH and/or a specific temperature which is optimal for the propagation of the first heterologous polypeptide. As such, in embodiments in which the yeast is from the genus Saccharomyces, the process can comprise controlling the pH of the culture medium to between about 3.0 to about 6.0, about 3.5 to about 5.5 or about 4.0 to about 5.5. In a specific embodiment, the pH is controlled at about 4.5. In another example, in embodiments in which the yeast is from the genus Saccharomyces, the process can comprise controlling the temperature of the culture medium between about about 20° C. to about 40° C., about 25° C. to about 30° C. or about 30° C. to about 35° C. In a specific embodiment, the temperature is controlled at between about about 30° C. to about 35° C. (32° C. for example).

At the end of the propagation process, a specific concentration can be sought or achieved. In some embodiments, the concentration of the propagated recombinant yeast host cell in the culture medium is at least about 0.1, 0.15, 0.2, 0.25, 0.3, 0.35, 0.4, 0.45, 0.5, 1.0, 1.5, 2.0, 2.5, 3.0, 3.5, 4.0, 4.5, 5.0 or more weight % with respect to the volume of the culture medium. In a specific embodiment in which the recombinant yeast host cell is propagated using a fed-batch process, the concentration of the propagated recombinant yeast host cell in the culture medium is at least about 0.25 weight% with respect to the volume of the culture medium.

The process can also include modifying the propagation medium obtained after the propagation step to provide a yeast composition. In an embodiment for providing a yeast composition, at least one component of the mixture obtained after propagation is removed from the culture medium to provide the yeast composition. This component can be, without limitation, water, amino acids, peptides and polypeptides, nucleic acid residues and nucleic acid molecules, cellular debris, fermentation products, etc. In an embodiment, the formulating step comprises substantially isolating the propagated recombinant yeast host cells from the components of the propagation medium. As used in the context of the present disclosure, the expression “substantially isolating” refers to the removal of the majority of the components of the propagation medium from the propagated recombinant yeast host cells. In some embodiments, “substantially isolating” refers to concentrating the propagated recombinant yeast host cell to at least 5, 10, 15, 20, 25, 30, 35, 45% or more when compared to the concentration of the recombinant yeast host cell prior to the isolation. In order to provide the yeast composition, the propagated recombinant yeast host cells can be centrifuged (and the resulting cellular pellet comprising the propagated recombinant yeast host cells can optionally be washed), filtered and/or dried (optionally using a vacuum-drying technique). The isolated recombinant yeast host cells can then be formulated in a yeast composition. In some embodiments, the process includes a step of adding a stabilizer (like a polyol, such as, for example, glycerol) to the yeast composition. The yeast composition can be provided in an active or a semi-active form. The yeast composition can be provided in a liquid, semi-solid or dry form. In an embodiment, the yeast composition can be provided in the form of a cream yeast. Once formulated in a yeast composition, the process can optionally include a step of storing the yeast composition prior to fermentation. The yeast composition can be stored for 1, 3, 4, 5, 6, 7, 8, 9, 9, 10 hours or more, for 1, 2, 3, 4, 5, 6, 7, 8, 9, 10 days or more.

The yeast composition can be optionally be supplemented with a stabilizer and/or stored prior to the fermentation phase. In such embodiment, the yeast composition can include, for example, one or more stabilizers or preservatives and, in some embodiment, a polyol, like, for example, glycerol.

Because the expression of the first heterologous polypeptide is limited or avoided during the propagation phase, the population of propagated recombinant yeast host cells, prior to fermentation, do not substantively express the first heterologous polypeptide. As such, the population of propagated recombinant yeast host cells or the yeast composition comprising same exhibits stability as it does not have a tendency to decrease its dry cell weight, decrease its internal trehalose content and/or produce a substantive amount CO₂ or ethanol during storage (which could limit the recombinant yeast host cell’s fermentative performance).

The yeast composition can be, in some embodiments, stored in a container comprising a minimal amount of 500, 600, 700, 800, 900, 1000 kg or more. In some further embodiments, the yeast composition can be provided in a minimal volume of 500, 600, 700, 800, 900, 1000 L or more. In some further embodiments, the yeast composition can be provided in a minimal volume of 125, 150, 175, 200, 225, 250 gallons or more. In yet some additional embodiments, the yeast composition can be provided at a density of at least 0.5, 0.6, 0.7, 0.8, 0.9, 1.0, 1.06 kg/L or more.

### Process for Making a Fermented Product

The recombinant yeast host cells described herein can be used to convert a biomass (present in the fermentation medium) into a fermentation product. The fermented product can be an alcohol, such as, for example, ethanol, isopropanol, n-propanol, 1-butanol, methanol, acetone and/or 1, 2 propanediol. The process comprises contacting the population of propagated recombinant yeast host cell or the yeast composition described herein under conditions to allow the conversion of at least a part of the biomass into the fermentation product. In some embodiments, the fermentation process can include providing a biomass which is different from the propagation medium. Alternatively, the fermentation process can be conducted in the propagation medium (which may be supplemented with a carbohydrate source for example).

The biomass that can be fermented with the recombinant yeast host cells described herein includes any type of biomass known in the art and described herein. For example, the biomass can include, but is not limited to, starch, sugar and lignocellulosic materials. Starch materials can include, but are not limited to, mashes such as corn, wheat, rye, barley, rice, or milo. Sugar materials can include, but are not limited to, sugar beets, artichoke tubers, sweet sorghum, molasses or cane. The terms “lignocellulosic material”, “lignocellulosic substrate” and “cellulosic biomass” mean any type of biomass comprising cellulose, hemicellulose, lignin, or combinations thereof, such as but not limited to woody biomass, forage grasses, herbaceous energy crops, non-woody-plant biomass, agricultural wastes and/or agricultural residues, forestry residues and/or forestry wastes, paper-production sludge and/or waste paper sludge, waste -water-treatment sludge, municipal solid waste, corn fiber from wet and dry mill corn ethanol plants and sugar-processing residues. The terms “hemicellulosics”, “hemicellulosic portions” and “hemicellulosic fractions” mean the non-lignin, non-cellulose elements of lignocellulosic material, such as but not limited to hemicellulose (i.e., comprising xyloglucan, xylan, glucuronoxylan, arabinoxylan, mannan, glucomannan and galactoglucomannan), pectins (e.g., homogalacturonans, rhamnogalacturonan I and II, and xylogalacturonan) and proteoglycans (e.g., arabinogalactan-polypeptide, extensin, and pro line -rich polypeptides).

In a non-limiting example, the lignocellulosic material can include, but is not limited to, woody biomass, such as recycled wood pulp fiber, sawdust, hardwood, softwood, and combinations thereof; grasses, such as switch grass, cord grass, rye grass, reed canary grass, miscanthus, or a combination thereof; sugar-processing residues, such as but not limited to sugar cane bagasse; agricultural wastes, such as but not limited to rice straw, rice hulls, barley straw, corn cobs, cereal straw, wheat straw, canola straw, oat straw, oat hulls, and corn fiber; stover, such as but not limited to soybean stover, corn stover; succulents, such as but not limited to, agave; and forestry wastes, such as but not limited to, recycled wood pulp fiber, sawdust, hardwood (e.g., poplar, oak, maple, birch, willow), softwood, or any combination thereof. Lignocellulosic material may comprise one species of fiber; alternatively, lignocellulosic material may comprise a mixture of fibers that originate from different lignocellulosic materials. Other lignocellulosic materials are agricultural wastes, such as cereal straws, including wheat straw, barley straw, canola straw and oat straw; corn fiber; stovers, such as corn stover and soybean stover; grasses, such as switch grass, reed canary grass, cord grass, and miscanthus; or combinations thereof.

Substrates for cellulose activity assays can be divided into two categories, soluble and insoluble, based on their solubility in water. Soluble substrates include cellodextrins or derivatives, carboxymethyl cellulose (CMC), or hydroxyethyl cellulose (HEC). Insoluble substrates include crystalline cellulose, microcrystalline cellulose (Avicel), amorphous cellulose, such as phosphoric acid swollen cellulose (PASC), dyed or fluorescent cellulose, and pretreated lignocellulosic biomass. These substrates are generally highly ordered cellulosic material and thus only sparingly soluble.

It will be appreciated that suitable lignocellulosic material may be any feedstock that contains soluble and/or insoluble cellulose, where the insoluble cellulose may be in a crystalline or non-crystalline form. In various embodiments, the lignocellulosic biomass comprises, for example, wood, corn, corn stover, sawdust, bark, molasses, sugarcane, leaves, agricultural and forestry residues, grasses such as switchgrass, ruminant digestion products, municipal wastes, paper mill effluent, newspaper, cardboard or combinations thereof.

Paper sludge is also a viable feedstock for lactate or acetate production. Paper sludge is solid residue arising from pulping and paper-making, and is typically removed from process wastewater in a primary clarifier. The cost of disposing of wet sludge is a significant incentive to convert the material for other uses, such as conversion to ethanol. Processes provided by the present invention are widely applicable. Moreover, the saccharification and/or fermentation products may be used to produce ethanol or higher value added chemicals, such as organic acids, aromatics, esters, acetone and polymer intermediates.

The process of the present disclosure contacting the recombinant host cells described herein with a biomass so as to allow the conversion of at least a part of the biomass into the fermentation product (e.g., an alcohol such as ethanol). In an embodiment, the biomass or substrate to be hydrolyzed is a lignocellulosic biomass and, in some embodiments, it comprises starch (in a gelatinized or raw form). The process can include, in some embodiments, heating the lignocellulosic biomass prior to fermentation to provide starch in a gelatinized form.

The fermentation process can be performed at temperatures of at least about 25° C., about 28° C., about 30° C., about 31° C., about 32° C., about 33° C., about 34° C., about 35° C., about 36° C., about 37° C., about 38° C., about 39° C., about 40° C., about 41° C., about 42° C., or about 50° C. In some embodiments, the process can be conducted at temperatures above about 30° C., about 31° C., about 32° C., about 33° C., about 34° C., about 35° C., about 36° C., about 37° C., about 38° C., about 39° C., about 40° C., about 41° C., about 42° C., or about 50° C.

The fermentation process can be conducted, at least in part, in the presence of a stressor (such as high temperatures or the presence of a bacterial contamination).

In some embodiments, the process can be used to produce ethanol at a particular rate. For example, in some embodiments, ethanol is produced at a rate of at least about 0.1 g per hour per liter, at least about 0.25 g per hour per liter, at least about 0.5 g per hour per liter, at least about 0.75 g per hour per liter, at least about 1.0 g per hour per liter, at least about 2.0 g per hour per liter, at least about 5.0 g per hour per liter, at least about 10 g per hour per liter, at least about 15 g per hour per liter, at least about 20.0 g per hour per liter, at least about 25 g per hour per liter, at least about 30 g per hour per liter, at least about 50 g per hour per liter, at least about 100 g per hour per liter, at least about 200 g per hour per liter, or at least about 500 g per hour per liter.

Ethanol production can be measured using any method known in the art. For example, the quantity of ethanol in fermentation samples can be assessed using HPLC analysis. Many ethanol assay kits are commercially available that use, for example, alcohol oxidase enzyme based assays.

The present invention will be more readily understood by referring to the following examples which are given to illustrate the invention rather than to limit its scope.

### EXAMPLE I

TABLE 1

Description of the strains used in this example. The heterologous genes were placed at neutral integration sites in the yeast’s genome.

| | |
|---|---|
| Strain | Genes overexpressed |
| M2390 | None, wild-type Saccharomyces cerevisiae parental strain |
| M15419 | Glucoamylase MP743 (SEQ ID NO: 32, 4 copies) FDH1 (SEQ ID NO: 3, 2 copies) STL1 (SEQ ID NO: 6, 2 copies) ADHE (SEQ ID NO: 2, 4 copies) PFLA (SEQ ID NO: 4, 2 copies) PFLB (SEQ ID NO: 5, 2 copies) |
| M19399 | Glucoamylase MP743 (SEQ ID NO: 32, 4 copies) |
| | ADHE (SEQ ID NO: 2; 4 copies) FDH1 (SEQ ID NO: 3, 2 copies) PFLA (SEQ ID NO: 4, 2 copies) PFLB (SEQ ID NO: 5, 2 copies) STL1 (SEQ ID NO: 6, 4 copies) |
| M20790 | Trehalase (SEQ ID NO: 7, 2 copies) under the control of the tir1 promoter (SEQ ID NO: 10) ADHE (SEQ ID NO: 2; 2 copies) FDH1 (SEQ ID NO: 3, 2 copies) PFLA (SEQ ID NO: 4, 2 copies) PFLB (SEQ ID NO: 5, 2 copies) STL1 (SEQ ID NO: 6, 4 copies) |
| M19481 | Glucoamylase MP1152 (SEQ ID NO: 34; 4 copies) Trehalase (SEQ ID NO: 7, 2 copies) under the control of the tef2 promoter (SEQ ID NO: 9) TSL1 (SEQ ID NO: 13, one copy) under the control of a modified tsl1 promoter (SEQ ID NO: 35) ADHE (SEQ ID NO: 2; 4 copies) PFLA (SEQ ID NO: 4, 2 copies) PFLB (SEQ ID NO: 5, 2 copies) FDH1 (SEQ ID NO: 3, 2 copies) STL1 (SEQ ID NO: 6, 4 copies) |
| M21211 | Glucoamylase MP1152 (SEQ ID NO: 34; 4 copies) Trehalase (SEQ ID NO: 7, 2 copies) under the control of the tir1 promoter (SEQ ID NO: 10) ADHE (SEQ ID NO: 2; 2 copies) PFLA (SEQ ID NO: 4, 2 copies) PFLB (SEQ ID NO: 5, 2 copies) STL1 (SEQ ID NO: 6, 4 copies) FDH1 (SEQ ID NO: 3, 2 copies) |
| M20398 | ADHE (SEQ ID NO : 2) 2 copies) PFLA (SEQ ID NO : 4, 2 copies) PFLB (SEQ ID NO : 5, 2 copies) FDH1 (SEQ ID NO: 3, 2 copies) STL1 (SEQ ID NO: 6, 4 copies) |
| M20576 | Glucoamylase MP743 (SEQ ID NO: 32, 4 copies) ADHE (SEQ ID NO : 2) 2 copies) GAPN (SEQ ID NO: 33, 2 copies) Trehalase (SEQ ID NO: 7, 2 copies) under the control of the tef2 promoter (SEQ ID NO: 9) STL1 (SEQ ID NO: 6, 4 copies) GAPN (SEQ ID NO : 33, 4 copies) TSL1 (SEQ ID NO: 13, one copy) under the control of a modified tsl1 promoter (SEQ ID NO: 35) |
| M23293 | Glucoamylase MP1152 (SEQ ID NO : 34, 4 copies) STL1 (SEQ ID NO : 6, 2 copies) GAPN (SEQ ID NO : 33, 4 copies) Trehalase (SEQ ID NO: 7, 2 copies) under the control of the tir1 promoter (SEQ ID NO: 10) |
| M10874 | Protease MP812 (SEQ ID NO: 37) under the control of the tef2 promoter (SEQ ID NO: 9) |
| M21757 | Protease MP812 (SEQ ID NO: 37) under the control of the tir1 promoter (SEQ ID NO: 10) |

### Yeast Propagation and Stabilized Liquid Yeast Production

The yeast were propagated and the SLY was made according to the details provided in U.S. Pat. 7,968,320 (incorporated herein it its entirety). Briefly, the propagation work included one L4-seed propagation and two L5-commercial propagations. All propagations were conducted with a 60%:40% raw sugars to water feeding mixture (w/w). Sugar blends included: 50% beet molasses, 35% cane molasses, and 15% brown syrup (Lantic cane). Propagation stages included batch pre-culture (50 mL media), batch pure culture (1400 mL media), fed-batch seed prop (AB3090B-L4), and fed-batch commercial props (AB3095C-L5). Each stage was carried out as follows.

Pre-culture (50 mL) – The pre-culture was started from glycerol stock cryovials. Under aseptic conditions, 17 µL of yeast slurry from the glycerol stock were transferred to a flask containing molasses media (50 mL of autoclaved leftover batch media -see composition below-). The flask was incubated for 48 h at 32° C. and 150 rpm (Innova-40 orbital incubator, New Brunswick Scientific, USA).

Batch (1400 mL) – Batch composition: 250 g molasses (50% beet molasses, 35% cane molasses, and 15% brown syrup), 15.72 g Fermaid K, and 7.84 g MAP. These ingredients were diluted with enough water (ca. 1500 mL) to produce an 11%-Brix solution (12 % Brix after autoclave). The initial pH (4.8) was adjusted with sulfuric acid. 1400 mL of the molasses solution were placed in a 2.8-L-flask, capped using a sterilization bio-shield wrap membrane (Kimberly-Clark, GA, USA), and autoclaved 45 minutes at 123° C. at 1 atm. The flask was inoculated with 3.8 mL taken from the previously incubated pre-culture and incubated for another 24 hours at 150 rpm and 32° C. (Innova-40 orbital incubator, New Brunswick Scientific, USA).

L4 seed propagation – The 1400 mL batch was used to inoculate the propagation vessel (Bailun Bio-Technology Co., Shanghai). Feed streams consisted of a 60% w/w sugar media and 5% ammonia. The propagation set water (5600 mL) included 18 mL of concentrated H₃PO₄, vitamins and minerals: 1875 mg thiamine, 675 mg CAP, 40 mg B6, 0.85 mg biotin, 35 mg nicotinamide, 87.5 mg CuSO₄·5H₂O, 17 mg MnSO₄, 30 mg FeNH₄(SO₄)₂·12H₂O, 50 mg CoSO₄·7H₂O, 12.5 mg H₃BO₃, 37.5 mg Na₂MoO₄2H₂O, 1700 mg ZnSO₄·7H₂O, 17 g MgSO₄·7H₂O. The initial pH was 4.8, adjusted with a 2 M NaOH solution. The pH set point of the recipe was 4.8 and was adjusted with automatic additions of sodium hydroxide or sulfuric acid (both 2 M solutions). Mixing and air rates were 800 rpm and 19 L min-1 respectively, with the temperature kept at 32° C. The 24-hour recipe used was AB3090B. The recipe targeted a specific growth rate of 0.18 h⁻¹.

L5 commercial propagations – An aliquot of 350 g Y30 of L4 yeast was used to inoculate each L5 propagation (Bailun Bio-Technology Co., Shanghai). Feed streams consisted of a 60% w/w sugar media and 5% ammonia. The propagation set water (ca. 5000 mL) included 18 mL of concentrated H₃PO₄, vitamins and minerals: 1875 mg thiamine, 675 mg CAP, 40 mg B6, 0.85 mg biotin, 35 mg nicotinamide, 87.5 mg CuSO₄·5H₂O, 17 mg MnSO₄, 30 mg FeNH₄(SO₄)₂·12H₂O, 50 mg CoSO₄·7H₂O, 12.5 mg H₃BO₃, 37.5 mg Na₂MoO₄·2H₂O, 1700 mg ZnSO₄·7H₂O, 17 g MgSO₄·7H₂O. The initial pH was 4.8 adjusted with a 2 M NaOH solution. The pH set point of the recipe increased from 4.5 to 5.8 and was adjusted with automatic additions of sodium hydroxide or sulfuric acid (both 2 M solutions). Mixing and air rates were 800 rpm and 19 L min-1 respectively with the temperature kept at 32° C. The commercial recipe AB3095C targeted a specific growth rate of 0.18 h⁻¹ and protein and phosphate (P₂O₅) concentrations of 32% and 1.8%, respectively. The final yeast product was treated with stabilizers for long-term storage in cold conditions.

### Aerobic and Anaerobic Growth of Yeast

Control strains M2390 or M19481 were plated onto YPD (1% yeast extract, 2% peptone, 4% glucose) agar plates and grown overnight at 35° C. Duplicate wells for each control strain was inoculated into 600 µl of YPD media in a 96 well deep dish culture plate using a tip size amount of cells from the patched plate. Eight colonies for each transformation (tef2p, tir1p, pau5p, or dan1p) were inoculated into the same 96 well plate as control strains. Two layers of porous film were adhered to the top of the plate and incubated aerobically at 35° C. for 48h, shaking at 900 RPM prior performing the trehalase assay. After 24 h of incubation, 20 µl of the culture was inoculated into a fresh 96 well plate which was placed into a glove bag devoid of oxygen to create an anaerobic environment. The plate was incubated for 72 h prior to performing the trehalase assay.

### Trehalase Assay

A 1% trehalose solution was made in 50 mM Sodium Acetate, pH 5.0. 10 µl of culture supernatant using the aerobic or anaerobic preparations was added to 50 µl of the 1% trehalose solution in a 96 well PCR plate. The aerobic cultures were incubated for 20 min at 35° C. and aerobic cultures for 2 h at 35° C. 100 µl of 3,5-dinitrosalicyclic acid (DNS) was added directly to each plate and boiled for 5 min at 99° C. in a PCR thermocycler. 75 µl of the reaction was transferred to a flat bottom plate and the absorbance measured at 540 nm.

### Construction of Mutant TSL1 Promoter

In order to increase the activity of the native tsl1p, an error prone PCR was performed on the native tsl1p. The primers were designed to amplify the native tsl1p and PCR mutagenesis was performed following the instructions using the Diversify PCR Random Mutagenesis Kit (Clontech, 630703). Four rounds of mutagenesis were performed using the previous PCR product as template to introduce additional mutations (Buffer 5 conditions, ~4bp mutations per kb per round). The mutant library was transformed into yeast using glucoamylase expression as a readout for promoter strength. Starch activity assays were performed to compare native tsl1p-glucoamylase activity with individual isolates. A promoter with 3 fold increase in starch activity was chosen for expression of tsl1 (e.g., having the nucleotide sequence of SEQ ID NO: 35).

### Corn Mash Fermentation

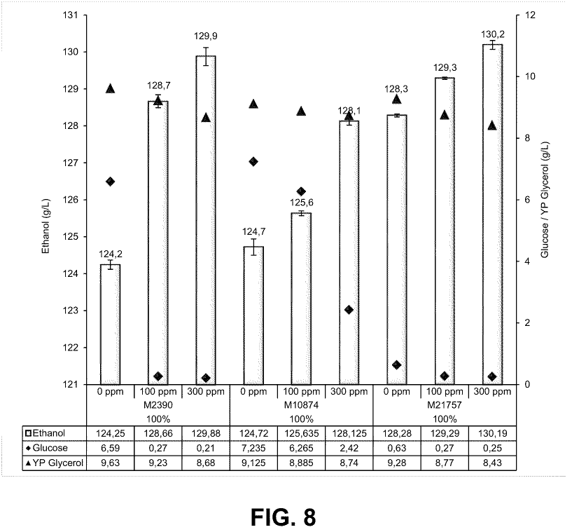

For the results presented on FIGS. 6 and 7, YPD cultures (25 to 50 g) were inoculated into 30-32.5% total solids (TS) corn mash containing lactrol (7 mg/kg) and penicillin (9 mg/kg) in 125 mL bottles fitted with one way valves. Urea was added at a concentration of 0-300 ppm. Exogenous glucoamylase was added at 100% = 0.6 A GU/gTS and 0-65% for strains expressing a glucoamylase. The strains were incubated at 33° C. for 18 h-48 h, followed by 31° C. for permissive fermentation, 36° C. hold for high temp or 34° C. hold for lactic fermentation, shaking at 150 RPM. 0.38% w/v lactic was added at T = 18 h. Samples were collected at 18-68 h depending on the experiment and metabolites were measured using high pressure liquid chromatography (HPLC). For the results presented on FIG. 8, YPD cultures were inoculated into 20.10% total solids (TS) of a corn mash and at a concentration of 0.05 g/L of dry cell weight. Urea was added, as indicated on the figure, at a concentration of 0-300 ppm. Exogenous glucoamylase was added at 100% = 0.6 A GU/gTS. The fermentations were carried out using 10 mL scintillation vials with a total sample size of 3 grams. The strains were incubated at 33° C. for 0-21 h, followed by 31° C. for 21-51 h. Samples were collected at 51 h depending on the experiment and metabolites were measured using high pressure liquid chromatography (HPLC).

Strains M15419 and M19481 were propagated and formulated as a SLY. Strain M19481, expressed a heterologous trehalase under the control of an aerobic promoter. Strain M15419 does not express a heterologous trehalase. At the end of the production, in the SLY obtained from M19481, trehalose was converted into glucose and a subsequent fermentation occured, causing release of CO₂ in the SLY (foaming) and difficulty toting. FIG. 1 shows an example of the “bubbling” observed in the SLY obtained from strain M19481. Foaming was not observed in the SLY obtained from strain M15419. In addition, a ten-fold increase in ethanol present in the supernatants of M19481 SLY compared to M15419 SLY, a non-trehalase expressing strain, was observed (Table 2).

TABLE 2

HPLC metabolites in SLY supernatant

| Strain | Glucose | Lactic | Glycerol | Acetic | Ethanol | DP4+ | DP3 | DP2 |
|---|---|---|---|---|---|---|---|---|
| M19481 | 0.21 | 2.46 | 55.06 | 0.51 | 27.7 | 0.00 | 0.30 | 0.09 |
| M15419 | 0.00 | 0.21 | 39.58 | 0.10 | 3.09 | 0.00 | 0.13 | 0.00 |

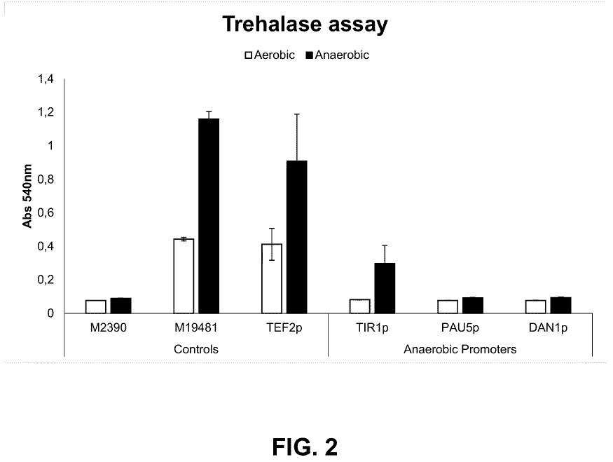

Three anaerobic promoters (tir1p, pau5p, and tir1p) were chosen for expression of the same heterologous trehalase (SEQ ID NO: 7) for comparison to the constitutive tef2p. Expression constructs were engineered into strain M20398. Eight transformants were grown aerobically or anaerobically and assayed for trehalase activity using trehalose as a substrate. Average activity for the eight colonies for each promoter is shown in FIG. 2 and is compared to wildtype strain, M2390 and M19481.

Strains M19399, M19481 and M20790 were selected for scale down propagation. Their respective dry cell weight, intracellular trehalose content, and ethanol were determined in SLY supernatant over time. Samples harvested were analyzed immediately for intracellular trehalose, dry cell weight and HPLC metabolites and then again after SLY stabilization and for eight days.

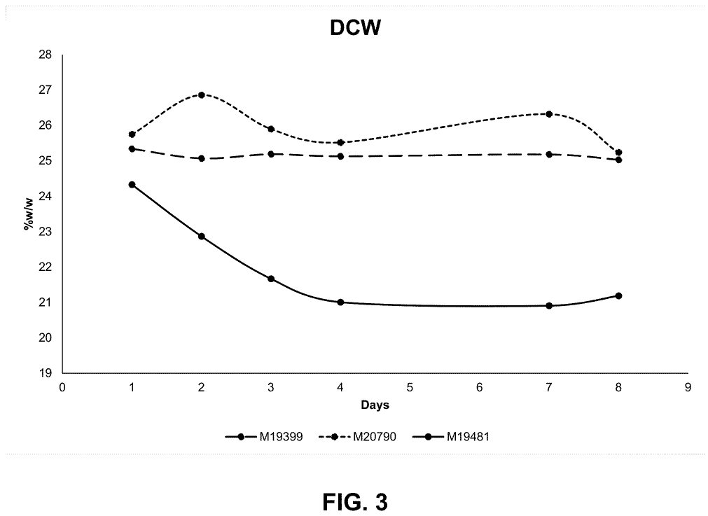

No foaming was observed for strains M19399 and M20790 during storage (data not shown). The SLY solids of strains M19399 and M20790 did not show a decline in weight over time (FIG. 3). However, a ~3% drop in the dry cell weight in the SLY obtained from M19481 was observed within the first three days of storage (FIG. 3).

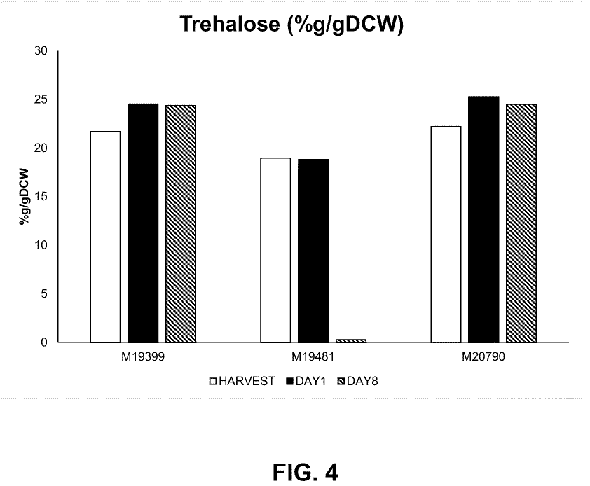

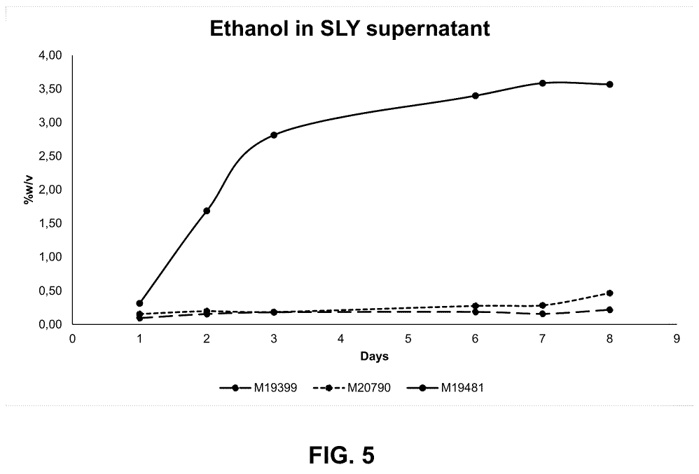

In parallel, a depletion in intracellular trehalose and an increase in the production of ethanol was observed for strain M19481 (FIGS. 4 and 5). In contrast, the intracellular trehalose as well as the ethanol content in the SLY supernatant for strains M19399 and M20790 remained constant during storage (FIGS. 4 and 5).

Strains M2390 (which does not express a heterologous trehalase), M15419 and M21211 were submitted to various laboratory scale corn fermentations under permissive as well as non-permissive fermentation (FIG. 6). The data presented in FIG. 6 shows no loss in robustness and additional ethanol yield increase with concomitant decrease in DP2 relative to the M15419 strain (FIG. 6).

Strains M2390 and M23293 were submitted to a lab scale permissive fermentation. At the end of the fermentation, strain M23293 produced more ethanol, less ethanol and DP2 (trehalose) and consumed more glucose than strain M2390 (FIG. 7).

Strains M2390, M10874 and M21757 were submitted to a lab scale corn mash permissive fermentation. At the end of the fermentation, strain M10874 produced less ethanol than control strain M2390 in the presence of 100 or 300 ppm of urea (FIG. 8). Strain M21757 produced more ethanol than control strain M2390 in the presence of 0, 100 or 300 ppm of urea (FIG. 8).

### EXAMPLE II

A total of 50 Saccharomyces cerevisiae promoters were individually fused to a nucleic acid molecule encoding a Saccharomycopsis fibuligera glucoamylase (SEQ ID NO: 1) or an Aspergillus niger xylanase (SEQ ID NO: 38) to allow for activity assays in recombinant Saccharomyces cerevisiae host cells. The expression cassettes used the same native IDP1 terminator sequence and were integrated at one copy per chromosome at a neutral integration site of the wild type yeast S. cerevisiae strain, M2390. Transformants were initially screened for activity and genotyped, with one isolate chosen based on the average activity of eight isolates. The single isolates were grown in 0.6 mL YP-glucose 40 g/L in 2 mL deep well 96-well plates for 48 h either aerobically or anaerobically. The cultures were centrifuged to remove the cells and the supernatant used in either a starch assay for the Saccharomycopsis fibuligera glucoamylase library or a birchwood xylanase assay for the Aspergillus niger xylanase library. The gelatinous corn starch assay was conducted using 50 µl of 1% gel starch along with 10 µl of yeast supernatant, incubated for 30 min at 35° C., then 100 µl of DNS added to each assay and boiled for 5 mins. A total of 50 µl of the DNS mix was transferred to a 96-well round bottom plate reader plate and analyzed at 540 nm. Similarly, the birchwood xylanase assay was conducted by adding 5 µl of supernatant to 45 µl of 1% birchwood xylan, incubated for 60 min at 35° C., and 75 µl of DNS added to each reaction and boiled for 5 mins. A total of 50 µl was transferred to a plate reader plate and the absorbance analyzed at 540 nm.

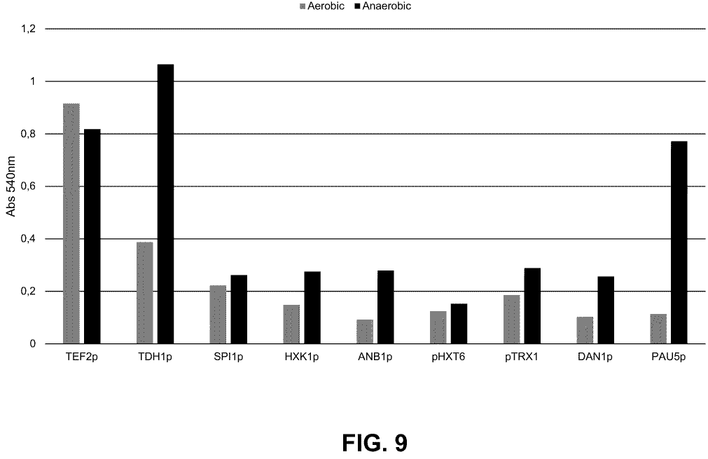

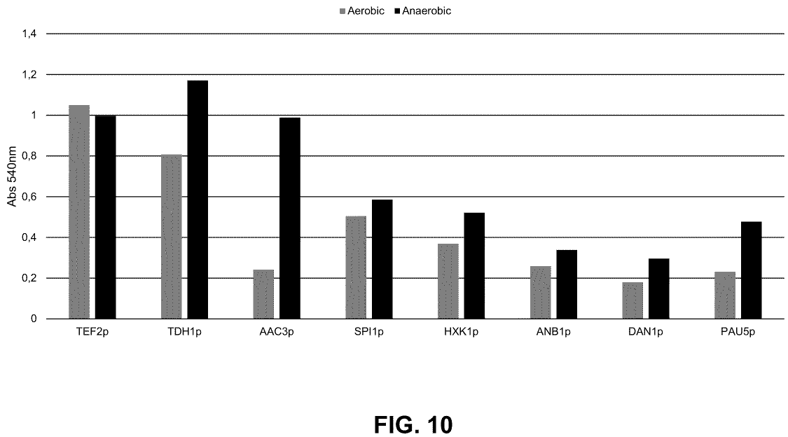

The S. cerevisiae native tef2 promoter (having the nucleic acid sequence of SEQ ID NO: 9) was used as a constitutive control for both assays. In FIGS. 9 and 10, the secreted activity of each gene is used as an expression proxy, with the activity correlated to the functionality of each promoter under each condition. Table 3 below provides the promoters which have been determined to preferentially express the glucoamylase or the xylanase in anaerobic conditions.

TABLE 3

Promoters used to express the Saccharomycopsis fibuligera glucoamylase or the Aspergillus niger xylanase under anaerobic conditions.

| Promoter | SEQ ID NO: |
|---|---|
| Form the tdh1 gene (tdh1p) | 39 |
| From the spi1 gene (spi1p) | 40 |
| From the hxk1 gene (hxk1p) | 41 |
| From the anb1 gene (anb1p) | 42 |
| From the hxt6 gene (phxt6) | 43 |
| From the trx1 gene (ptrx1) | 44 |
| From the dan1 gene (dan1p) | 12 |
| From the pau5 gene (pau5p) | 11 |
| From the aac3 gene (aac3p) | 45 |

While the invention has been described in connection with specific embodiments thereof, it will be understood that the scope of the claims should not be limited by the preferred embodiments set forth in the examples, but should be given the broadest interpretation consistent with the description as a whole.

### REFERENCES

An MZ, Tang YQ, Mitsumasu K, Liu ZS, Shigeru M, Kenji K. Enhanced thermotolerance for ethanol fermentation of Saccharomyces cerevisiae strain by overexpression of the gene coding for trehalose-6-phosphate synthase. Biotechnol Lett. 2011 Jul;33(7):1367-74.

Bell W, Sun W, Hohmann S, Wera S, Reinders A, De Virgilio C, Wiemken A, Thevelein JM. Composition and functional analysis of the Saccharomyces cerevisiae trehalose synthase complex. J Biol Chem. 1998 Dec 11;273(50):33311-9.

Cao TS, Chi Z, Liu GL, Chi ZM. Expression of TPS1 gene from Saccharomycopsis fibuligera A11 in Saccharomyces sp. W0 enhances trehalose accumulation, ethanol tolerance, and ethanol production. Mol Biotechnol. 2014 Jan;56(1):72-8.

Ge XY, Xu Y, Chen X. Improve carbon metabolic flux in Saccharomyces cerevisiae at high temperature by overexpressed TSL1 gene. J Ind Microbiol Biotechnol. 2013 Apr;40(3-4):345-52.

Guo ZP, Zhang L, Ding ZY, Shi GY. Minimization of glycerol synthesis in industrial ethanol yeast without influencing its fermentation performance. Metab Eng. 2011 Jan; 13(1):49-59.

Thevelein JM, Hohmann S. Trehalose synthase: guard to the gate of glycolysis in yeast? Trends Biochem Sci. 1995 Jan;20(1):3-10.

What is claimed is:

1. A recombinant yeast host cell capable of expressing a first heterologous polypeptide under the control of a heterologous promoter, wherein:
the first heterologous polypeptide is an hydrolase; and
the heterologous promoter is capable of limiting the expression of the first heterologous polypeptide during a propagation and favoring the expression of the first heterologous polypeptide during a fermentation;
wherein the hydrolase is capable of generating an enzymatic product from a substrate and wherein the substrate is a yeast cellular component.

2. The recombinant yeast host cell of claim 1, wherein the yeast cellular component is an intracellular component, a component associated to a yeast cell membrane and/or a component associated to the yeast cell wall.

3. The recombinant yeast host cell of claim 1, wherein the hydrolase is a glycoside hydrolase.

4. The recombinant yeast host cell of claim 3, wherein the first heterologous polypeptide is a trehalase.

5. The recombinant yeast host cell of claim 4, wherein the trehalase:
(a) has the amino acid sequence of any one of SEQ ID NO.: 7 or 14 to 21;
(b) is a variant of the amino acid sequence of (a) exhibiting trehalase activity; or
(c) is a fragment of the amino acid sequence of (a) or (b) exhibiting trehalase activity.

6. The recombinant yeast host cell of claim 1, wherein the hydrolase is a peptide hydrolase.

7. The recombinant yeast host cell of claim 6, wherein the first heterologous polypeptide is a protease.

8. The recombinant yeast host cell of claim 7, wherein the protease has the amino acid sequence of SEQ ID NO: 37, is a variant of the amino acid sequence of SEQ ID NO: 37 exhibiting protease activity or is a fragment of the amino acid sequence of SEQ ID NO: 37 exhibiting protease activity.

9. The recombinant yeast host cell of claim 3, wherein the first heterologous polypeptide comprises a glycogen phosphorylase and/or a glycogen debranching enzyme.

10. The recombinant yeast host cell of claim 1, wherein the hydrolase is a glucan hydrolase.

11. The recombinant yeast host cell of claim 10, wherein the first heterologous polypeptide is a glucanase.

12. The recombinant yeast host cell of claim 1, wherein the cleavage of the substrate and/or the accumulation of the enzymatic product, if generated prior to the fermentation, is detrimental to the performance of the recombinant yeast host cell during the fermentation.

13. The recombinant yeast host cell of clim 1 being capable of modifying the enzymatic product into an inhibitory product detrimental to the performance of the recombinant yeast host cell during the fermentation, if generated prior to the fermentation.

14. The recombinant yeast host cell of claim 1 being capable of expressing:
one or more second heterologous polypeptide, wherein the one or more second heterologous polypeptide is a saccharolytic enzyme;
one or more third heterologous polypeptide for modulating the production of formate;
one or more fourth heterologous polypeptide for converting acetyl-CoA into an alcohol;
one or more fifth heterologous polypeptide involved in the production of glycerol, in the regulation of the production of glycerol or in the transport of glycerol;
one or more sixth heterologous polypeptide involved in producing trehalose and/or in regulating trehalose production; and/or
one or more seventh heterologous polypeptide having glyceraldehyde-3-phosphate dehydrogenase activity.

15. The recombinant yeast host cell of claim 1, wherein the heterologous promoter is an anaerobic specific promoter.

16. The recombinant yeast host cell of claim 15, wherein the anaerobic specific promoter comprises a promoter from the tir1 gene, the pau5 gene, the dan1 gene, the tdh1 gene, the spi1 gene, the hxk1 gene, the anb1 gene, the hxt6 gene, the trx1 gene and/or the aac3 gene.

17. The recombinant yeast host cell of claim 1 being from the genus Saccharomyces sp. from the species Saccharomyces cerevisiae.

18. A process for obtaining a population of propagated recombinant yeast host cells, the process comprising contacting the recombinant yeast host cell claim 1 with a propagation medium under conditions so as to allow the propagation of the recombinant yeast host cell to obtain the propagated recombinant yeast host cell population.

19. A yeast composition comprising a population comprising the recombinant yeast host cells of claim 1 and a stabilizer.

20. A process for converting a biomass into a fermentation product, the process comprises contacting the biomass with the recombinant yeast host cell of claim 1 under conditions to allow the conversion of at least a part of the biomass into the fermentation product.


SEQUENCE LISTING (56 sequences)

SEQ ID NO: 1 - protein, 515 aa, Saccharomycopsis fibuligera
  SIGNAL 1..27
MIRLTVFLTAVFAAVASCVPVELDKRNTGHFQAYSGYTVARSNFTQWIHEQPAVSWYYLLQNIDYPEGQFKSAKPGVVVASPSTSEPDYFYQWTRDTAITFLSLIAEVEDHSFSNTTLAKVVEYYISNTYTLQRVSNPSGNFDSPNHDGLGEPKFNVDDTAYTASWGRPQNDGPALRAYAISRYLNAVAKHNNGKLLLAGQNGIPYSSASDIYWKIIKPDLQHVSTHWSTSGFDLWEENQGTHFFTALVQLKALSYGIPLSKTYNDPGFTSWLEKQKDALNSYINSSGFVNSGKKHIVESPQLSSRGGLDSATYIAALITHDIGDDDTYTPFNVDNSYVLNSLYYLLVDNKNRYKINGNYKAGAAVGRYPEDVYNGVGTSEGNPWQLATAYAGQTFYTLAYNSLKNKKNLVIEKLNYDLYNSFIADLSKIDSSYASKDSLTLTYGSDNYKNVIKSLLQFGDSFLKVLLDHIDDNGQLTEEINRYTGFQAGAVSLTWSSGSLLSANRARNKLIELL

SEQ ID NO: 2 - protein, 910 aa, Bifidobacterium adolescentis
MADAKKKEEPTKPTPEEKLAAAEAEVDALVKKGLKALDEFEKLDQKQVDHIVAKASVAALNKHLVLAKMAVEETHRGLVEDKATKNIFACEHVTNYLAGQKTVGIIREDDVLGIDEIAEPVGVVAGVTPVTNPTSTAIFKSLIALKTRCPIIFGFHPGAQNCSVAAAKIVRDAAIAAGAPENCIQWIEHPSIEATGALMKHDGVATILATGGPGMVKAAYSSGKPALGVGAGNAPAYIDKNVDVVRAANDLILSKHFDYGMICATEQAIIADKDIYAPLVKELKRRKAYFVNADEKAKLEQYMFGCTAYSGQTPKLNSVVPGKSPQYIAKAAGFEILEDATILAAECKEVGENEPLTMEKLAPVQAVLKSDNKEQAFEMCEAMLKHGAGHTAAIHTNDRDLVREYGQRMHACRIIWNSPSSLGGVGDIYNAIAPSLTLGCGSYGGNSVSGNVQAVNLINIKRIARRNNNMQWFKIPAKTYFEPNAIKYLRDMYGIEKAVIVCDKVMEQLGIVDKIIDQLRARSNRVTFRIIDYVEPEPSVETVERGAAMMREEFEPDTIIAVGGGSPMDASKIMWLLYEHPEISFSDVREKFFDIRKRAFKIPPLGKKAKLVCIPTSSGTGSEVTPFAVITDHKTGYKYPITDYALTPSVAIVDPVLARTQPRKLASDAGFDALTHAFEAYVSVYANDFTDGMALHAAKLVWDNLAESVNGEPGEEKTRAQEKMHNAATMAGMAFGSAFLGMCHGMAHTIGALCHVAHGRTNSILLPYVIRYNGSVPEEPTSWPKYNKYIAPERYQEIAKNLGVNPGKTPEEGVENLAKAVEDYRDNKLGMNKSFQECGVDEDYYWSIIDQIGMRAYEDQCAPANPRIPQIEDMKDIAIAAYYGVSQAEGHKLRVQRQGEAATEEASERA

SEQ ID NO: 3 - protein, 292 aa, Bifidobacterium adolescentis
MSEHIFRSTTRHMLRDSKDYVNQTLMGGLSGFESPIGLDRLDRIKALKSGDIGFVHSWDINTSVDGPGTRMTVFMSGCPLRCQYCQNPDTWKMRDGKPVYYEAMVKKIERYADLFKATGGGITFSGGESMMQPAFVSRVFHAAKQMGVHTCLDTSGFLGASYTDDMVDDIDLCLLDVKSGDEETYHKVTGGILQPTIDFGQRLAKAGKKIWVRFVLVPGLTSSEENVENVAKICETFGDALEHIDVLPFHQLGRPKWHMLNIPYPLEDQKGPSAAMKQRVVEQFQSHGFTVY

SEQ ID NO: 4 - protein, 791 aa, Bifidobacterium adolescentis
MAAVDATAVSQEELEAKAWEGFTEGNWQKDIDVRDFIQKNYTPYEGDESFLADATDKTKHLWKYLDDNYLSVERKQRVYDVDTHTPAGIDAFPAGYIDSPEVDNVIVGLQTDVPCKRAMMPNGGWRMVEQAIKEAGKEPDPEIKKIFTKYRKTHNDGVFGVYTKQIKVARHNKILTGLPDAYGRGRIIGDYRRVALYGVNALIKFKQRDKDSIPYRNDFTEPEIEHWIRFREEHDEQIKALKQLINLGNEYGLDLSRPAQTAQEAVQWTYMGYLASVKSQDGAAMSFGRVSTFFDVYFERDLKAGKITETDAQEIIDNLVMKLRIVRFLRTKDYDAIFSGDPYWATWSDAGFGDDGRTMVTKTSFRLLNTLTLEHLGPGPEPNITIFWDPKLPEAYKRFCARISIDTSAIQYESDKEIRSHWGDDAAIACCVSPMRVGKQMQFFAARVNSAKALLYAINGGRDEMTGMQVIDKGVIDPIKPEADGTLDYEKVKANYEKALEWLSETYVMALNIIHYMHDKYAYESIEMALHDKEVYRTLGCGMSGLSIAADSLSACKYAKVYPIYNKDAKTTPGHENEYVEGADDDLIVGYRTEGDFPLYGNDDDRADDIAKWVVSTVMGQVKRLPVYRDAVPTQSILTITSNVEYGKATGAFPSGHKKGTPYAPGANPENGMDSHGMLPSMFSVGKIDYNDALDGISLTNTITPDGLGRDEEERIGNLVGILDAGNGHGLYHANINVLRKEQLEDAVEHPEKYPHLTVRVSGYAVNFVKLTKEQQLDVISRTFHQGAVVD

SEQ ID NO: 5 - protein, 376 aa, Saccharomyces cerevisiae
MSKGKVLLVLYEGGKHAEEQEKLLGCIENELGIRNFIEEQGYELVTTIDKDPEPTSTVDRELKDAEIVITTPFFPAYISRNRIAEAPNLKLCVTAGVGSDHVDLEAANERKITVTEVTGSNVVSVAEHVMATILVLIRNYNGGHQQAINGEWDIAGVAKNEYDLEDKIISTVGAGRIGYRVLERLVAFNPKKLLYYDYQELPAEAINRLNEASKLFNGRGDIVQRVEKLEDMVAQSDVVTINCPLHKDSRGLFNKKLISHMKDGAYLVNTARGAICVAEDVAEAVKSGKLAGYGGDVWDKQPAPKDHPWRTMDNKDHVGNAMTVHISGTSLDAQKRYAQGVKNILNSYFSKKFDYRPQDIIVQNGSYATRAYGQKK

SEQ ID NO: 6 - protein, 569 aa, Saccharomyces cerevisiae
MKDLKLSNFKGKFISRTSHWGLTGKKLRYFITIASMTGFSLFGYDQGLMASLITGKQFNYEFPATKENGDHDRHATVVQGATTSCYELGCFAGSLFVMFCGERIGRKPLILMGSVITIIGAVISTCAFRGYWALGQFIIGRVVTGVGTGLNTSTIPVWQSEMSKAENRGLLVNLEGSTIAFGTMIAYWIDFGLSYTNSSVQWRFPVSMQIVFALFLLAFMIKLPESPRWLISQSRTEEARYLVGTLDDADPNDEEVITEVAMLHDAVNRTKHEKHSLSSLFSRGRSQNLQRALIAASTQFFQQFTGCNAAIYYSTVLFNKTIKLDYRLSMIIGGVFATIYALSTIGSFFLIEKLGRRKLFLLGATGQAVSFTITFACLVKENKENARGAAVGLFLFITFFGLSLLSLPWIYPPEIASMKVRASTNAFSTCTNWLCNFAVVMFTPIFIGQSGWGCYLFFAVMNYLYIPVIFFFYPETAGRSLEEIDIIFAKAYEDGTQPWRVANHLPKLSLQEVEDHANALGSYDDEMEKEDFGEDRVEDTYNQINGDNSSSSSNIKNEDTVNDKANFEG

SEQ ID NO: 7 - protein, 692 aa, Neurospora crassa
  SIGNAL 1..22
MVSRFLGATVPLAAAILPGARALYVNGSVTAPCDSPIYCYGELLHQVELARPFSDSKTFVDMPTIKPVDEVLEAFSKLTLPLSNNSELHEFLSTYFGPAGGELEAVPTDQLHVSPTFLDNVSDDVIKQFVDSVINIWPDLTRKYVGAGELCTGCADSFIPVNRTFVVAGGRFREPYYWDSFWILEGLLRTGGAFTEISKNIIENFLDLVEQIGFVPNGARLYYLDRSQPPLLTQMVRIYVEHTNDTSILERAVPVLKKEWEWWTTNRTVEVTADGKTYSLQRYHVDNNQPRPESYREDYITANNNSYYATSGIIYPETTPLNDTQKALLYANLASGAESGWDYSSRWLKNPGDAARDVYFPLRSLNVLEIVPVDLNSILYQNEVTIGKFLAQQGSKDEAEEWAKKAEERSEAMYKLMWNSTLWSYFDYNLTSSSQNIYVPADPQVFPFEQPSGTPEGYQVLFSVNQMFPFWTGAAPDQLKGNPLAVKLAFERIKNLLDNKAGGIPATNFVTGQQWDEPNVWPPLMHVLMDGLLNTPATFGEDDPAYQETQTLALRLAQRYVDSTFCTWYATGGSTSETPKLQGLGSDLKGIMFEKYSDNSTNVAGSGGEYEVVEGFGWTNGVLIWAADKFGDKLKRPDCGDITPAQVGKRADITMEKRAVELDVFDAKFTKKFARKGKLEKLKAKFKRRAAI

SEQ ID NO: 8 - DNA, 2076 bp, synthetic construct
  misc_feature 1..2076: note = Encoding SEQ ID NO: 7 and codon-optimized for Saccharomyces cerevisiae expression
  source 1..2076: mol_type = other DNA
atggtcagtagatttttgggtgctactgttccattggctgctgctattttgccaggtgctagagcattatatgttaacggttctgttactgctccatgcgattctccaatctactgttatggtgaattattgcaccaagtcgaattggctagaccattctctgattctaagacctttgttgatatgccaaccatcaagccagttgatgaagttttggaagctttctctaagttgaccttgccattgtctaacaactccgaattgcatgaattcttgtctacttactttggtccagctggtggtgaattggaagctgttccaactgatcaattgcatgtttctccaactttcttggacaacgtttccgatgatgttatcaagcaattcgttgactccgttattaacatttggccagatttgaccagaaagtatgttggtgccggtgaattgtgtactggttgtgctgattctttcatcccagttaacagaacttttgttgttgctggtggtagattcagagaaccatattactgggattctttctggatcttggaaggtttgttgagaactggtggtgctttcactgaaatctccaagaacattatcgaaaactttttggacttggtcgaacaaatcggttttgttccaaatggtgctagattgtactacttggatagatctcaaccaccattattgacccaaatggttagaatctacgttgaacataccaacgacacctccattttggaaagagctgttcctgttttgaagaaagaatgggaatggtggactaccaacagaactgttgaagttactgctgatggtaagacctactcattgcaaagataccacgttgacaacaatcaacctagaccagaatcttacagagaagattacattaccgccaacaacaactcttactatgctacctctggtatcatctacccagaaactactccattgaacgatactcaaaaggctttgttgtacgctaatttggcttctggtgctgaatctggttgggattattcttctagatggttgaagaatccaggtgatgctgctagagatgtttactttccattgagatccttgaacgtcttggaaatcgttccagttgatttgaactccatcttgtaccaaaacgaagttaccatcggtaagttcttggctcaacaaggttctaaagatgaagctgaagaatgggctaaaaaggccgaagaaagatctgaagctatgtacaagttgatgtggaactctactttgtggtcctacttcgattacaacttgacctcttcttctcaaaacatctacgttccagctgatccacaagtttttccatttgaacaaccatctggtactccagaaggttaccaagttttgttctccgtcaatcaaatgtttccattctggactggtgctgctccagatcaattgaaaggtaatccattagctgttaagttggccttcgaaagaatcaagaacttgttggataacaaggccggtggtattccagctactaattttgttactggtcaacaatgggatgaacctaatgtttggccaccattgatgcatgttttgatggatggtttattgaacactccagctacctttggtgaagatgatccagcttatcaagaaactcaaaccttggctttgagattggctcaaagatacgttgattctactttctgtacttggtatgctactggtggttctacttctgaaactccaaaattgcaaggtttgggttctgatttgaagggtatcatgttcgaaaagtactccgataactctacaaacgttgctggttcaggtggtgaatatgaagttgttgaaggttttggttggaccaacggtgttttgatttgggctgctgataagtttggtgacaagttgaaaagaccagattgcggtgatattactccagctcaagttggtaaaagagccgatattactatggaaaagagagccgttgaattggacgtttttgatgctaagttcaccaagaagtttgccagaaagggtaaattggaaaagttgaaggccaagttcaaaagaagagctgccatt

SEQ ID NO: 9 - DNA, 499 bp, Saccharomyces cerevisiae
  source 1..499: mol_type = genomic DNA
gggcgccataaccaaggtatctatagaccgccaatcagcaaactacctccgtacattcctgttgcacccacacatttatacacccagaccgcgacaaattacccataaggttgtttgtgacggcgtcgtacaagagaacgtgggaactttttaggctcaccaaaaaagaaaggaaaaatacgagttgctgacagaagcctcaagaaaaaaaaaattcttcttcgactatgctggagccagagatgatcgagccggtagttaactatatatagctaaattggttccatcaccttcttttctggtgtcgctccttctagtgctatttctggcttttcctatttttttttttccatttttctttctctctttctaatatataaattctcttgcattttctatttttctctctatctattctacttgtttattcccttcaaggttttttttaaggactacttgtttttagaatatacggtcaacgaactataattaactaaac

SEQ ID NO: 10 - DNA, 750 bp, Saccharomyces cerevisiae
  source 1..750: mol_type = genomic DNA
cgctcaacactttactccatcttcttaaagggtgtaaacagaacgataaatgatttccataaatagtaaggcccagccgacagcctgcagcgcaaaagtaaaccgtttccatctaatttggataatctgcattgttcccaacgaatgacagaatccatcgccagatctaataaggttcaattttctctatacgacggctataaaaggaagttttccgagcgcgcttcgaagcagaatagggaggaccccatacccgttttggtgcctgtccctttttagtacgtgtttccgtttccgtgcctgtccctttttagtacctgtttccgtttccgtgcctggctagatccatctttcttcgcgcgtttattttcagcaccatgttttaggtttttacagcatcgtttaaggaacccaacaatacaatagcgggaagaatgcactttctcgttccataaagggtctctttcacctatacggttggtacagatttccagtgtatgccagtcagccacggcattacgtcgtttgcttctattttcttcgtttggaactgcgtttgtatgcaactgtcccttgacagagaaaaaaagtgaagcaaaatgacagacaaagaaatcttttgtataaaaggtcggttgaatcttgttgttagcttggaatcagcttgcttttctcctctaaattacataaaaaaccaagaaaatatcagactttttcattcgctttcaacaagtactacaataattaaaa

SEQ ID NO: 11 - DNA, 750 bp, Saccharomyces cerevisiae
  source 1..750: mol_type = genomic DNA
atacgaatcagatactgttcggtacacgatatctaattaaaatgattcaaaactttgtaacaggtaaagttttcactagaaccaatcaatccaagtgaattaggggaaaccatagttgttgatttgtagaaacctcacattgtacattgttggtttgttgggcatatcagaacgagagattttccaacattcaatatacactaaaccctatgacgagtcccacagatggcgtaaggtttttatgatttcagcagggtacgacgactagtaccatattaacattttttagtgtttctaatttgggaaaaggtccgtgttttttctcctagcaaccgtttagtgccaagggttaggcaattgaacgaggccaagacaatattggctttgcttctattacttggctaacattgtgtctgcaggtcgaaaggcacctttactgtaaggaacattcttgcgctctaaacatacgaagatatggggaatatgaagcgtgtttcttatacgaagtgcagcatcgttcaaggaaaatacatccccatagtaataatggctaagtggccaggaattagaatatgtgagatatgagtgcaaaatgagtgaccagtaatagcctgtttgggatgtaattgctcaaaaaatttatataaatacagcggtttgatcagctttgtttgagacatttctctgttcttttccttccagttaagcttatatctccactaagcaacaacccaaaaaacaacaaataca

SEQ ID NO: 12 - DNA, 750 bp, Saccharomyces cerevisiae
  source 1..750: mol_type = genomic DNA
attaagcagtagggtattttgtttgcaagaaaataaagctcaaaatatctttggagtttgacaatagtatagaaaaaatagctcagcaaccttaataaaaaaagggtcattgggcaaagcgttttaaaaagatttaagtggtagtgtttaaagtgaaagagattgatatagttaaaaattgttgagctcaattcacgctggattcggcgatccgttttcttcaatcctcacgtgctttcttcgtttgagtgcaaaagttcatatgatgctatctcccgcttatcttattagtcgaaaatggggagaatttcctattttatctgtcgtttagcacatatggccaggaaaatacataaggtttcgccgaacgacggggtcaattcgtcctttttgtacacatcgtttaatttatgaggaaaaattgatgaacgtatcctccgtagacgctcctctgaaaagtttcatgtttcctgcgcgttcctttgataggcaataaaacaatacaacgcgtgcctttgaaaatgccaagatctatacgaggcctctaacaaaacatcgttcaggaacagagaatgctaaaaatgcaaaagggtccctgggtactcattgaatagaaatgattgaaaatactgcgtataaaatagcacgactaaatgatactatttttatgtcgacacggtactatttcttcttttgcagataaaagtgtagcagataaaagtgtagcatactaaatatataccccaagta

SEQ ID NO: 13 - protein, 1098 aa, Saccharomyces cerevisiae
MALIVASLFLPYQPQFELDTSLPENSQVDSSLVNIQAMANDQQQQRALSNNISQESLVAPAPEQGVPPAISRSATRSPSAFNRASSTTNTATLDDLVSSDIFMENLTANATTSHTPTSKTMLKPRKNGSVERFFSPSSNIPTDRIASPIQHEHDSGSRIASPIQQQQQDPTANLLKNVNKSLLVHSLLNNTSQTSLEGPNNHIVTPKSRAGNRPTSAATSLVNRTKQGSASSGSSGSSAPPSIKRITPHLTASAAKQRPLLAKQPSNLKYSELADISSSETSSQHNESDPDDLTTAPDEEYVSDLEMDDAKQDYKVPKFGGYSNKSKLKKYALLRSSQELFSRLPWSIVPSIKGNGAMKNAINTAVLENIIPHRHVKWVGTVGIPTDEIPENILANISDSLKDKYDSYPVLTDDVTFKAAYKNYCKQILWPTLHYQIPDNPNSKAFEDHSWKFYRNLNQRFADAIVKIHKKGDTIWIHDYHLMLVPQMVRDVLPFAKIGFTLHVSFPSSEVFRCLAQREKILEGLTGADFVGFQTREYARHFLQTSNRLLMADVVHDEELKYNGRVVSVRFTPVGIDAFDLQSQLKDGSVMQWRQLIRERWQGKKLIVCRDQFDRIRGIHKKLLAYEKFLVENPEYVEKSTLIQICIGSSKDVELERQIMIVVDRINSLSTNISISQPVVFLHQDLDFSQYLALSSEADLFVVSSLREGMNLTCHEFIVCSEDKNAPLLLSEFTGSASLLNDGAIIINPWDTKNFSQAILKGLEMPFDKRRPQWKKLMKDIINNDSTNWIKTSLQDIHISWQFNQEGSKIFKLNTKTLMEDYQSSKKRMFVFNIAEPPSSRMISILNDMTSKGNIVYIMNSFPKPILENLYSRVQNIGLIAENGAYVSLNGVWYNIVDQVDWRNDVAKILEDKVERLPGSYYKINESMIKFHTENAEDQDRVASVIGDAITHINTVFDHRGIHAYVYKNVVSVQQVGLSLSAAQFLFRFYNSASDPLDTSSGQITNIQTPSQQNPSDQEQQPPASPTVSMNHIDFACVSGSSSPVLEPLFKLVNDEASEGQVKAGHAIVYGDATSTYAKEHVNGLNELFTIISRIIED

SEQ ID NO: 14 - protein, 1051 aa, Aspergillus fumigatus
LPNNNDRVARSLKRHGGHGHKQVDTNSSHVYKTRFPGVTWDDDHWLLSTTTLDQGHYQSRGSIANGYLGINVASVGPFFELDVPVSGDVINGWPLYSRRQTFATIAGFFDYQPTTNGSNFPWLNQYGGESVISGIPHWSGLILDLGDGNYLDATVDNKTITDFRSTYDFKSGVLSWSYTWTPKCNKGSFNITYRLFAHKLHVNQAVVDMEITPSQGSEATVVNVIDGFSAVRTDFVESGQDNGALFSAVRPWGISNVTAYVYTNLTASAGVDLTSRALVNDKPYVHSNESSIAQAVDVKFRANETVRITKFVGAASSDAFPNPQQTAKQAVSAAMGAGYMGSLQSHVEEWASILLDGSVDSFVDPATGKLPDDDHILNSQIIAVANTYYLLQNTVGKNAIKAVSGAPVNVDSISVGGLTSDSYAGLVFWDADVWMQPGLVASHPEAAQRVTNYRTKLYPQALENINTAFTSSKNRTTFSPSAAIYPWTSGRFGNCTGTGPCWDYQYHLNGDIGLSLMYQWIASGDTKTFREQHFPIYDSVATMYSNIVQRNGSSWTLTNMTDPDEYANHIDAGGFTMPLISETLSYANSFRKQFGLEQNETWTEISENVLLIREDGVTLEYTTMNGTAVVKQADIVLVTYPLVYDNNYTAQHALNDLDYYANQQSPDGPAMTWAIFAITANDVSPSGCSAYTYHQDSYDPYMRAPFYQLSEQMIDDAGINGGTHPAYPFLTGHGGANQVVLMGYLGLRLLPDDAIHIDPNLPPQVSNLKYRTFYWRGWPISSSSNRTHTTISRAANLAPLDTADSRFANASIPVLVGDPSNSTAYRLPVTAPLVVPNRQIGFNNTIPGNMVQCRPVYSPNDYAPGQFPIAAVDGATSTKWRPSTANMSSLTVALADVEINSKVSGFHFNWWQAPPVNATVIFHDEMLEDPVAAMSSSHGNSRYRVVTTLTNIEQSQPYDAQSTDNNEVVLNTGNTTDVSLSQTVHTSRYATLLISGNQAGGEEGATVAEWAILGESKGSSSGHGNNKRRLDVRAAAALSALNDRRYRQFNA

SEQ ID NO: 15 - protein, 1052 aa, Neosartorya udagawae
LPNNNGRIARSLKRHGGHGQKQVDTNSSHVYDTRFPGVTWDDDHWLLSTTTLDQGHYQSRGSIANGYLGINVASVGPFFELDVPVGGDVINGWPLYSRRQTFATIAGFFDYQPATNGSNFPWLNQYGGESVISGIPHWSGLILDLGNGTYLDATVDNKTITDFRSTYDFKSGVLSWSYTWTPTCNKGSFNITYRLFAHKLHVNQAVVDMEITPSQGSQATVVNVIDGYSAVRTDFVESGQDNGAIFSAVRPWGISNVTAYVYTNLTASAGVDLSSRALVNDKPYVHSNESSIAQAVNVKFSANETIRITKFVGAASSDAFPNPQQTAKQAVSAAMGAGYMGSLQSHVAEWASILLDGSVDSFVDPATGKLPDDEHILNSQIIAVANTYYLLQNTVGKNAIKAVSGAPVNVNSISVGGLTSDSYAGLVFWDADVWMQPGLVASHPEAAQRVTNYRTKLYPQALENINTAFTSSKNQTSFSPSAAIYPWTSGRFGNCTGTGPCWDYQYHLDGDIGLSLMYQWIASGDTQTFREQHFPIYDSIATMYSNIVQRNGSSWTLTNMTDPDEYANHVDGGGFTMPLISETLGYANSFRKQFGLEQNETWAEISENVLVIRENGVTMEYTTMNGTTVVKQADVVLVTYPLVYDNNYTAQDSLNDLDYYANRQSPDGPAMTWAIFAITANDVSPSGCSAFTYHQNSYDPYMRAPFYQLSEQMLDEASINGGTHPAYPFLTGHGGANQVVLFGYLGLRLLPDDAIHIDPNLPPQVSNVAYRTFYWRGWPISASSNRTHTTISRAANVEPLDTADSRFANATISVLVGDPSNSTAYQLPATGPLVVPNRQIGFNNTIPGNMVQCRPVYSPHDYVPGQFPIAAVDGATSTKWQPSTANMSSLTVALADIEINSKVSGFHFNWWQAPPVNATVIFHDEVLEDPVAAMSSAHGNSQYKIVTTLTNIEQSQPYNAQGTDYNVVAMSTGNTTEVNLSQTVHTSRYATLLISGNQGGGEKGATVAEWAILGESKGSSSGHGNNKRRLDVRAAAALSGGLNDRRWQQFNA

SEQ ID NO: 16 - protein, 775 aa, Aspergillus flavus
LPSQGTQNKHNPRVAKILKRHEGSSQKAKDSNNVYETKFDGVTWDEENWLLKTTTLDQGHYQSRGSVANGYLGINVASVGPFFELDEEVDGDVINGWPLYSRRQSFATIAGFFDSQPTTNGTNFPWLSQYGWDTAISGVPHWSGLILDLGDDVYLDSTVDDSTITDFQSTYDFKAGVLSWSYTWSPADKGSFEITYRLFANKLNITQAVVDMEIIPSVDANATVANVIDGYSAVRTDFVESGQDDGALFSAVRPWGISNVTAYIYTNLTGSANVDLSSRALVTGKPYVNTNESSVAQTVNVKFTAKEPVRITKFVGGASTDAFADPKQTAKEAASAALAAGYKNSLESHASEWANIMHENSVDRFTDPTTGKLPEDQHVIDSAVIAVTNIYYLLQNTVSQNAIAAVSNATVNETSFSVGGLTSDSYGGQVFWDADVWMQPGLVASHPEAAQGVTNYRVAKYQQAKENVKTAFTSSKNQTRFDPSAAIYPWTSGRAGNCTATGACFDYQYHLNGDIGLSMIYQWVASGDTEYFQEKHFPIYDSVATLYSNLVERNGSSWTLTNMTDPDEYANHVDAGGFTMPLIAQTLENANTFRQQFNLEPNDTWTEISENVLLLRQNNVTLEYTSMNGTAVVKQADVVLVTYPLAYESNYTAEMALSDLDYYANKQSADGPAMTWAIFSIVASDVSPSGCSAWTYHQYSYDPYTRGPFFQLSEQMLDNASINGGTHPAYPFLTGHGGANQVVLFGYLGLRLLPEEGIYITPNLPPQIPYVKYRT

SEQ ID NO: 17 - protein, 1022 aa, Fusarium oxysporum
TSTHDHDRIKKCYQRHGTSSDSRKASNNIYKTSFPGVTWDNDNWLLTTTNLDQGHYQSRGSVANGYLGINVAAVGPFFEIDADEEGGVINGWPLFSRRQTFATIAGFYDAQPKTNGTNFPWLLQYGYESVISGVPHWGGLIIDLGDDVYLDATVDNRTVHNFTSTYDFKAGVLEWSYTWEPKGKGSYQIKYRLFAHKLHVNQAIVDLTIVPSTDSKAKVVNVIDGYSAVRSDFVKSGQDEDGGIFSAVRPVGIANVTAYIYAQVNGSKSLDLSRRKLVHGKPYVHTNESSIAQAIPVKFSAGVPVHITKYVGAASSDAFEDPEKTAKEASHRALEEGYEKSLLSHLREWESVMPSDSVDSYAFPENDTLPDDEYIIDSAIITVTNTYYLLQNTVGKNAQKAVSGAPVNIDSISVGGLTSDSYAGLIFWDADLFMQPGLTTSHPEAAQRITNYRVAKYDQAKKNIATSFAGSQNKTKFSESAAVYPWTSGRFGNCTATGPCWDYEYHLNGDIGISLVNQWVTSGDTDFFKETLLPIYDSVANLFADLLKPNGSSWTITNMTDPDEYANHIDAGGFTMALASETLIQANQIRRQFGMTENKTQDEIASDVLFIRENGITLEFTTMNGSAIVKQADVVLMSFPLGYNDNYTDQNGLDDLDYYANKQSPDGPAMTWAIYSIVADELSPSGCSAYTYAQYSYKPYTRPPFYQLSEQLVDNATVNGGTHPAYPFLTGHGGANQVTIFGYLGLRLIPDQGLHVNPNLPPQIGYLKYRTFYWRGWPISAWSNYTHTTISRHPTTKPLDVADSRYANKGIAVYAGKMGDSALHHLTFDDPVVIKNRQIGSVNTVHGNLAQCRPVKSSNSYEPGQFPIAAVDGATSTKWQPSKAADVSSLTVSLAKKDVGSKVKGFYFDWADAPPINVTVLFHNKTIDDPTKVYGTSSHDSGYDVVVSIKKVKLSDAYNAKTDNLDAVVMPTGNTTNVTLPETVPLSRYATLLIAGNQALDKVDLKAGNGTGATVAEWAILH

SEQ ID NO: 18 - protein, 1033 aa, Escovopsis weberi
LESFQDRVSGCVNRHSSGSHPAPSKNVYQTSFDGVTWDQDNWMLSTTELQQGAFESRASVANGYLGINVAGAGPFFELDSDEPGGVINGWPLFSRRQTFATIAGFWDSQPLTEGRNFPWLSQYGGDSAISGVPHWGGLLLDLGNGEILDADVDAETISDFQSTYDFKAGVMTWSYKWTPASRKKTGPIGITYRLFAHKLNVNQAVVDLEIVAPKGAHSLSATVASVLDGYSAVRTDFVGSGRDGDSIYSAVRPVGIADVEAYVYAQISGSHGVDMSRKRLVSSHGSPYVRSNDSSVVETVPVSVSAGQTVRVTKFVGAASSDAFPDPRSTARTAVLDAAKAGFDALLKSHAAEWAEVLPEDSVDSFADPETNKLPQDDILVTDAIMAVVNTFYLLQNTVGKNAIEAACDAPLNVDSISVGGLASDSYAGQVFWDADLFMGPGLFTSHPDAAQRISNYRVKLYDQAKANAQTGFTSSQNETHIPAEAAAYAWMSGRFGNCTATGPCFDYEYHLNGDIGLSFVNQWVVSGDTEYFKETLFPIYDSMATLYASLLKRNGSYWTLTNMTDPDEYANNVDAGGFTMPLIAEMLRNANSFRQQFGLPQNETWNEMAENVLTLRENGVTLEFTTMNNSAVVKQADVIMLTFPLSYTDNYTTENSLNDLDYYALEQSPDGPAMTYAYFSIIANQISPSGCSAYTYAQNAFLPYLRGPWFQLSEQQVDNATINGGTHPAYPFLTGHGGANQVVIFGYLGLRLLPDDILHINPNLPPQVPYVRYRDFFWRGHAISAWSNATHTTLSRAARTTPLDTADARFDTSPITIYVGDADHPTVYKLPPKGSVVVPNRQAGFVATKEGNLVQCKPAISHDDIMPGQFPIAAIDGASSTKWQPASADKLSSMTVSFDKRDVGSLVSGFYFEWAQAPPVNATVVFHDELLSTSGKIPSGKGIVAQLSNIKPSKPFNVTAAQLDIIAMPESNTTEVTLKHPVPATRYASLYIIGNQKLSAADVEAKNGTGATVAEWAILGEEKEGCGPKRLI

SEQ ID NO: 19 - protein, 1030 aa, Microsporum gypseum
ETDAERNAGVFARNSALKKGSSGSEQPVYATRFKGVTWDVANWRLTTTELDQGHYQSRGSIANGYLGINVAAVGPFFELDVPVSGDVINGWPVFSRRQTFATISDFYSFQQSINATNFPWLNKYGGDLISGVPHWSGLILDLGDGNFLDATVQNSTISNFTSTLDMKGGILTWQYTWSPEKHNGTYDIFYQLVAHKLHVNQALVRMEITPSKDGNVSVVNVIDGYSAVRTDFKGSGQDGGAIYTSVNPEGISNVTAFIYAEMSGTEGVNLSSSSLVNDKPYLHTNGSTIAQSVNVKLRAGQTTKIDKFVGAATTDQFKNPRQAAKDASARALRTGYEESLKTHIAEWTTVFPSDSTEDYTIPGKKWLPLDHHIIEASIVSVVNPYYLLQSTASHNALTAVKNAPLNRGSIAVGGLTSDSYGGLIFWDADIWMQPGLVVAFPEASQIFSNYRVDKYGQALRNAQTQDLSSKKKTYFSPDAAVYPWTSGRFGKCTATGPCFDYQYHLNGDIGMQIVNNWVTTGDTEYFKSKLFPVYNSIATFFSQLVEKNGTQWTVTNMTDPDEFANLVDGGGYTMPLIATTLKYANQFREMFGLGANQTWSEIAQNVQVSRDPASQITLEYTTMNGSTQVKQADIVLNTFPLRYTEDYTHDNALRDLDYYAAKQSPNGPAMTYAIFSIVANEVSPSGCSAYTYGQYSFSPYVRAPFFQFSEQVVDDWSINGGTHPAYPFLTGNGGANQVAVFGYLGLRLVSDGILHLNPNLPPQIPHIRYRTFYWHGWPFEASANYTQTTIQRATNRRPLASADPKFANAPITVHVGPESNITVYSLPPSGQLVIPNRRSGSINTLEGNLVQCQPVYSPNEFAPGQFPISAVDGAASTKWQPRRASSTSSLTVSLPDDASSASISGFAFDWAQAPPISAKVVLHDEPLPPVMDAEDDAGNGFSHATPPGSVTVWETPEVPQSHPYDPITIDLNMIMTYKGNTTNITLPSAVPATKFATLLIRGNQALGPAEVKAGNGTGATVAEWSILRST

SEQ ID NO: 20 - protein, 1053 aa, Aspergillus clavatus
FQTNNHARVTRSLKRHAGHGHTPPTDTNSSNIYETRFPGVTWDNDNWVLATTTLDQGHYQSRGSVANGYLGINVASVGPFFELDTPVSGDVINGWPLFSRRQSFATIAGFFDFQPTTNGSNFPWLNQYGGESVISGVPHWSGLVLDLGDDTYLDAAVDNETISGFQSAYDFKSGVLSWSYTWTPTDDKGSFNITYRLFANKLHINQAVVDMEITPSQESQATVVNVIDGYSAVRTDFVESGEDDGAIFSAVRPWGIANVTAYVYANLTASKNVDLASHTLVADKPYIHTNESSVAQAVRVNFRANETVRITKFVGAASSDAFPDPQKTAKQAVSAALGAGYMESLQSHVAEWADILLDGSVDSFVDPVTGKLPDDEHIVNSQVIAVANTYYLLQNTVGKNATTAVSDAPVNVDSISVGGLTSDSYAGQVFWDADVWMQPGLVASHPEAAQRITNFRVVQYQQALENVNTAFTGSKNQTSFSPSAAIYPWTSGRFGNCTGTGPCWDYQYHLNGDIGLSLMYQWITSGDTKVFREQHFPIYDSIATLYSNLVERNGSSWTLTNMTDPDEYANHIDAGGFTMPLISETLGYANTFRKQFGHEQNETWSKIAENVLVIRENDVTLEYTTMNGTTVVKQADVVLVTYPLVYDNNYTSEYSLNDLDFYANKQSPDGPAMTWAIFAITANDVSPSGCSAYTYHQNSYDPYMRAPFFQLSEQTIDDASINGGTHPAYPFLTGHGGANQVVLFGYLGLRLLPDDAIHIDPNLPPQIPNVAYRTFYWHGWPISASSNRTHTTISRATKIAPLDTADPRFANVSIPVLVGYDTNATAYHLPPSGPLTVRNRQIGLNNTIPGNIIQCRPVYSPDDYAPGQFPIAAVDGATSTKWQPATTNTSALTVTLPDAEVNSVVSGFHFDWWQAPPVNATVIFHDETLEDPVTALSSSHGNPQYTVITTLTNIELSQPYNAESSDLNKVAMPTGNTTDVQLSSTVHAARYATLLISGSQGDGDAGATVAEWAILGQEKESSGHDNGKRRLDVRSAAALSGSLDDRRARRFTA

SEQ ID NO: 21 - protein, 788 aa, Metarhizium anisopliae
ANGKDRVAKCLARYSGQDRGRNRTTVYKTDFPGVTWDDDNWLLSTTTLEQGRYQSRGSVANGYFGISVASVGPFFELDAEDEGGDVINGWPLFSRRQSFATIAGFWNAQPETNGTNFGWLLQYGYESVISGVPHWSGLVLDLGNGVYLDSTVDNKTITNFRSTYDFKAGVLSWSYTWSPSAGNNGSYDIRYLMFTNKLHISQAVVDLEIVPSVDANATVVNVLDGYSAVRTDFVQSGEDAGAIYSAVRPTGIANVTAYIYANMTGSDDVDIGRKTLVSNKPYIRKNESSIAQAVPVTFSAGKAVRITKYVGAASGDAFDDPQQVAKNAASSALSQGFYKSLRSHVQEWDDVMPDHSVDSYADPDNGTLPQDSYIIDSAIIAVANTYYLLQSTVGPNAQSLVKDAPVNVDSISVGGLVSDSYAGLIFWDADLFMQPGLVVSHPQSAERITNYRVNKYGQAKANAQTSYTSSQNKTVFSKDAAAFPWTSGRFGNCTATGPCWDYQYHLNGDIGISFVNQLVATGDTRYFNESLFPVYDSIATLFSNLLAPNGSSWTVKNMTDPDEYANHVDAGGYTMPLIAETLQTANTFREQFGLEKNATWDSMATNVLFLRENGVTLEFTTMNGSAVVKQADVILNTFPLSYTTNYTTQESLNDLDYYANKQSPDGPAMTWAFFSIIANDISPSGCSAYTYSQYSYKPYARAPFYQLSEQLIDNATINGGTHPAYPFLTGHGGANQVNVFGYLGLRLLPDDTLHINPNLPPQLSHLRYRTFYWRGWPFAASSNATHTT

SEQ ID NO: 22 - protein, 963 aa, Ogataea parapolymorpha
MAQPDYFDDQTESYYLQDERVLGTTKFNQLNKYTYQPYVSNGYIGSRIPNLGFGFSYDQNENLTSSDLSNGWPLFNPRYAGSFIAGFFDAQPNTTGVNFPELRENGYESVISAVPQWTALQLAATLNGETYVLDPSTANTSSAHVTDYRQELRMATGTVSTAYTWLGAVTVNITVMAHRDFETLGLVQLEVAPVSGAAPLKLDVVDVLDFASTQRCVLESIGYDDAGIFITVQPEGVAYKHASLYSRLNVNASCINETLAAAFHKVTNTVSLVLEHPLSVTKYVGVVSDDLLGTNSSDATLAAAKRTALDAAKYSWPSLRTMHDNAWADVWGDVAVEVENEPYLTLAAEASIYHLFANTRSSARNLTAALSVGGLSSDSYGGLVFWDADLWMIPALLPIAPETSVALNSYRYYLHEQAVRNAAANSYSGAVYPWTSGRFGNCTGTGPCINYEYHLNGAICYSVWKAYLSGAINDEHLEQYGWPVLRDAADFFADYVRYNDTLQKYTTHNLTDPDEYANFKDNAAYTAVVISQVMKWADRVARHLGKPSNSTQLKIMENMYLPQSRDNITLEYDTMNSSVLIKQADVVLIPYIDDEDGALAQNFGYDEVRATNDLSYYSLHQSSQGPAMTFPVFAAVSQKLNDYGCGSQTYHYKSVAPFLRFPFAQMSEQNNDNYDANGGTHPAFPFNTAHGGLVQSYFFGLTGIRFSYAVTPEHRLQRVLHFDPVELPLFSGDLKISGFKYLNQSLEIVIGETNGTIRHRGTAESILVYVDDRNAAAGYYTLEPGTELTVPVYVKQFNTPGSLTECQALAHSLTPGRDGDVIMSIIDGDNSTTWQAENKNGNAAVLLEFETTETFNAGAIVWGNRPAANFSLSVVAEPLDTTGTDVVIDETKLVRVLTDHVVQIASPYNASDTEVRIAEPNSTIFALPQEYTAQYVLLEVYGTLDTDDSTYGASVAELGLFYH

SEQ ID NO: 23 - protein, 1157 aa, Kluyveromyces marxianus
MIIIPLVVLVFTVLAPVYFYVTKPESSTHSLFPELAPARISWPFAGTCASSSGGEEDPLYCPDAYRKASEKMYDLLKDNEYAFYDETSETLGNLLLSENTFSRQPYVANGYIGSRIPNVGFGFAYDAINIWVNDSAIPGALNNGWPLRNQRYAGSFVSDFYSLQEKLNSTNFAELDKDGYSTVISSIPDWTDLSIMIHRGPGENNVEYINPTDVKLDKITDYMQNLSMRDGIVTTKFVYDNNLFVTTRTLAHRSIYPLGIVDMEIELLPQATENGLHEASVELEICDTFNFTTSHRTVLADFGHDKKNEGIYMIVEPENVPYSNASMFSYFDIPSRDEYTVAKTNDSVSQCTRRVLTTDSRENSTFIVRKFTGIVSSEYDNNNPEHMSNLERATAVVMENKGDYKNLLKMHRDHWKRLYADASIEIPSDGLLEMTAKSSIYHLLANSRSHNVSQSRGLPVPPSGLSSDSYGGMVFWDADVWMLPALLPFFPEIAKQMSAYRNASLAQAKENAKKYGLQGAIFPWTSGRFANCTSTGPCVDYEYHINVDIALSSLYIYMSGEEDEEKSEEYLRYTTWPFIENAAKMFTDYVKWNDTLQQYTTHNLTDPDEFANHVDNGAFTNAGIKSIMGWAHDIANHLGLDPDPKWTEIAEKIHIPISDTNITLEYTGMNSSVDIKQADVVLMTYPLGYFTETSQPRNAIKDIYYYSERQSASGPAMTYPVFVAASASLLNSGSSSQSYLYKSVVPYLRSPFAQFSEQSDDNFLTNGLTQPAFPFLTANGGYLQSILFGLTGLRYSYEVDKDTGKMHRLLKFNPISLPMFPGGIRINNFKYMGQVLDILLTDNEGIIKHKKGNKSILIKIPDRGDIPDVKPDEYTQINGTSVNVKRAVPSGESYHTIEPGTVFKTPLYNPKRNMANNIVESKRATNITVGVPGDVAVSAIDGNNYTHWQPANKKQPGRILIDMGNGTANEIKSGKILWGNRPAKSFSLSILPQFDQITQNMTSVLSQPSSHNCSNDDGWDSNCKYQEEEENIDAAIKDVFEWYGMDLQSVIENYPELSNVSMGFIKLVDHYNVTPSYPWKNVNSTRIELTLGNETNFVVDYSKVPELNLNNNLGVDLQSKDTRWRKPRFVVLTVFDTYDDDDEVKGATIKELSLFDN

SEQ ID NO: 24 - protein, 1038 aa, Komagataella phaffii
MPYGSIYNSRIPKKPPPTSQTREMLNRVLLVALSCVVFFHLVTTFPVGTSSDSLQIRNLLSHNFTRANISEGLSSGATYFVDEDTETYYDKELKVLRTTRFPRYNNYQLQPYVANGYIGSRIPRVGSGFTYDTSDNKTSENLKNGWPLFNKRYSGAFIAGFFNSQPTVPETNFEELEKDGYESIIASIPQWTSLELTVNVNGTNQTLKADDVDITHISDYSQQLSLLDGIVTTNYTWLGLVNVSISVLAHRDIVSLGFVSLELSSQKNITVSVTDILDFATSTRCSYLDSGVNEQSIFMKVQPSNVPTNATIYSSLMSSNSTSSLLKQNQTVSQTLRVNLSKNQAASFQKYVGVVSDDYLDSIETNLTSYQFARETAKFAEIKGRSWILKSHKEAWNELLNGKSIVFHDNDFLTLASDSSIYHLMANTRSEANGGTSALGVSGLSSDSYGGMVFWDTDFWMLPSVQAFSPRHAVSLSKFRDHTHDQAKKNAQTRDMNGAVYPWTSGRFGNCTSTGPCYDYEYHINIDIAFMFWKLYLGGAIDDDYMKEFGYPIIEDVASFFVDYVDYNSTLDKYTTRNLTDPDEYAEFKNNAAFTNVGISQLMKWALILGKHLKVGNERSYDKWEDIMTKMYLPVNHAGDVTLEYTGMNNSIEVKQADVVLISYPLDDEDGALQEYFDYDEDRAISDVRYYSDKQTDEGPAMTFSVYSAVNAKFNKEGCSSQTYLLKSVEPYFRFPFGQMSEQSTDQYDTNGGTHPAFPFLTGHGAFLQSSIYGLTGLRFSYIYNDTDKSIKRRLAFDPLQLPCLPGGFSINGFVYMNQTLDITVNDTYATIAHRGNATTINVYVDSRNEMGGKEHKIQPGKSLSIPLYQTEQNIPGSFIECTVKNVTALQPGVVGDPIQAVADGDNSTIWKIESREEPTHLIFDLGDELDIEGGLVVWGTYPAESFSVSVLRDFNSTNYRVINNVENYDLIYESGNVTASSPFDESHIKKVQILPHNCTNFTFSELTASRYVLFEFTDVLGYPQDYSYGAQVAEVVL

SEQ ID NO: 25 - protein, 1180 aa, Ashbya gossypii
MADTASLPPQRDSALGMHGPHGGLYMPVAQGPLQAHASPRLVSVRMVLSSITALALVAVVTVLGTAQPARPTAPLAAADEQFWVAQHRSASKQLYQLVHGSELSFYDEGRDVLGTTELSRNMYSRQPYVANGYIGSRVPNVGFGYAADEENIWTDASVPGALNNGWPLRNPRYAGSFVSDFYSLQARLNSTNFPELDEEGYSTVIASIPEWTDLRVRADGAELGAETVALEDMGGYVQNMSLADGVVTTEYVWRGLSVRATVAAHRSEYPLGLVQLEVALCGDTEPREVEVRDVLNFTTSHRTVLREAGHDEDGIYMRVEPENVPYSEAALYSVFEVRGGEGSVQPERAAAGATVAQWVRVRLTAAQPRVVVRKYVGVVSSEYNTAGGSNLEAARAAALAHYGAFDGALVSHRAAWSALYGNASIEIPSDFLLELAAKSSMFHMLANTRAHNVSATRGLPVPVTGLSSDSYGGMVFWDSDVWMLPGLLPFFPDIAREISNYRNATHAQAVANARHYNYSGALYPWTSGRYANCTSTGPCVDYEYHINIDIAMSSLSIYMNGADGIGEDYLRYTTWPLLRDAALFFTEYVRYNETLDAYTTHNLTDPDEFANFIDNGAFTNAGIKILLRWAIDVGTHLEEPVDTKWQEISDKIHIPTSETNITLEYTGMNATVDIKQADVLLMVYPLGYITDESILNNAIQNLYYYSERQSASGPAMTYPVFAAAAATLLNHGSSSQSYLYKAVVPYLRAPFFQFSEQSDDNFLTNGLTQPAFPFLTANGGYLQSLLFGLTGLRYSYTVNPETKKMERLLKFSPVRMPLLPGGIRINNFKYLGQVLDISIDDHNATIAHKQGNTPIHIKVPDRSILRDRDVPVYKGSALQARDVIPYHELSNSNYFTVNPGETLTLPVYEPELNIQGNIVEGRQITNLTQGVPGDVPISILDGNNYTHWQPFDKSERALLLIDLGSEEEYEITTGKILWGARPAKNFSISILPNSKHITEILTKLTAMMDGRNTDLVSCSKCHAVSSSQHLLGGLANVTDSKGLAAIDGETVDMGIREIFRWNLFDLPTISSIIPEAANISESFVTVLENYQVTPSEPYYEEVVRKSQIVILPSNETDFCIDYAAVPKLNPTYTAVNLSADDTNWRKTRFVIVAVEGSYDDDDDQKGGTIKEIALMVAPKN

SEQ ID NO: 26 - protein, 674 aa, Thielavia terrestris
LYINGSVTAPCDSPLYCHGEILKAIELAHPFTDSKTFVDMPTIRPLDEVIAAFNRLSQPLSNNSELNAFLAANFAPAGGELEAVPRDQLHTEPSFLNKLDDTVIKEFVAKVIDIWPDLTRRYGGPGNCTACANSFIPVNRTFVVAGGRFREPYYWDSYWILEGLLRTGGAFTEISKNIIENFLDFVETIGFIPNGARIYYLDRSQPPLLARMVRSYVDYTNDTSILDRALPLLIKEHEFWSTNRSVSIKAPNGKTYTLNRYYVNNNQPRPESFREDYITANNGSYYAASGIIYPVNTPLNDTEKAELYANLASGAETGWDYSTRWLKNPNDAAKDIYFPLRSLNVRGTVPVDLNSILYENEVIISQYLKRAGNNSEAERWAYAASQRSEAMFELMWNATHWSYFDYNLTSNSQRIFVPVDDDATAAERAGAPRGQQVLFNIGQFYPFWTGAAPAQLKNNPLAVQQAYARVARMLDENAGGIPATNFVTGQQWDQPNVWPPLQHVLMEGLLNTPPTFGDADPAYQSVRALALRLAQRYLDSTFCTWYATGGSTSQTPQLQGVAPGAEGIMFEKYADNSTNVAGSGGEYEVVEGFGWSNGVLIWAADVFGAQLKRPDCGNITAAHTSGSGAQKRSGGSLARRAVELDPWDAAWTKMFGRSALKKREDVRKRWLLAA

SEQ ID NO: 27 - protein, 1052 aa, Aspergillus lentulus
SPNNNDRIARSLKRHGGHGHKQADTNSSHVYKTRFPGVTWDDDHWLLSTTTLDQGHYQSRGSIANGYLGINVASVGPFFELDVPVGGDVINGWPLYSRRQTFATIAGFFDYQPTTNGSNFPWLNQYGGESVISGIPHWSGLILDLGDGNYLDATVDNKTITDFRSTYDFKSGVLSWSYTWTPRCNKGSFDITYRLFAHKLHVNQAVVDMEITPSQGSEATVVNVIDGYSAVRTDFVESGQDSGALFSAVRPWGISNVTAYVYTNLTASAGVDLSSRALVNDKPYVHSNESSVAQAVNVKFRANETVRITKFVGAASSDAFPNPQQTAKQAVSAAMGAGYMGSLQSHVAEWASILLDGSVDSFVDPATGKLPDDDHILNSQIIAVANTYYLVQNTVGKNAIKAVSGAPVNVNSISVGGLTSDSYAGLVFWDADVWMQPGLVASHPEAAQSVTNYRTKLYPQALENINTAFTSSKNQTSFSPSAAIYPWTSGRFGNCTGTGPCWDYQYHLNGDIGLSLMYQWVASGDTKTFREQHFPIYDSVATVYSNLVQRNGSSWTLTNMTDPDEYANHVDGGGFTMPLISETLSYANSFRKQFGLEQNETWTEISENVLVIRENGVTMEYTTMNGTTVVKQADVVLVTYPLVYDNNYTAQYALNDLDYYANKQSPDGPAMTWAIFAITANDVSPSGCSAYTYHQDSYDPYMRAPFYQLSEQMIDDASINGGTHPAYPFLTGHGGANQVVLMGYLGLRLLPDDAIHIDPNLPPQVSNVKYRTFYWRGWPISASSNRTHTTISRAANIAPLDTADSRFANATISVLVGDPSNSTAYRLPATGPLVVPNRQIGFNNTIPGNMVQCRPVYSPHDYAPGQFPIAAVDGATSTKWRPATANMSSLTVTLADVEINSKVSGFHFDWWQAPPVNATVIFHDEMLEDPVAAVSSAHGNSRYKVVTTLTNIKQSQPYNAESTDYNEVVMATGNTTDVNLSQTVHTSRYATLLISGNQAGGKEGATVAEWAILGESKGSSSGHGNNKRRLDVRAAAALSGGLNDRRYRQFNA

SEQ ID NO: 28 - protein, 1066 aa, Aspergillus ochraceoroseus
MRVTQLLVKGSGLQSKNLRLSGCMRRHGGGHGIEKTLVGSNNTYQTVFPGVSWDDDHWLLTTTTPDPGHYQSRGSVANGYIGISVSSIGPFFELDMPVDGDVISGWPLFSRRQSFATIAGFYDYQPTTNGSNFPWINQYGGESVISGVPHWSGLIIDLGDETYLDSTVDNQTITGFSSTYDFKAGMLSWSYTWTPAAGDKGSYKITYRIFANKLNVNQAVVDMEIIPSIDSEAIIVNVLDGYAAVRTDFVSSGQDDGAIYSAVRPWGIENVTAYIYANITGSDAVHLSSRRIVTGKAYVNINESSIAQAVDVKFSASEKVRITKFVGAASTDAFSDPQQTAKQAVSQALTAGYLRCLQSHVAEWASIMPDNSVDRFVNPSTGKLPDDQNIISSAIISVTNPYYLLQNTVGKKAIREASDAPLNVDSLSVGGLVSDSYAGLVFWDADVWMQPGLVASHPEAAQRVTNYRTEKYAQAKANAKTTFAGSKNKTYVEPSAAVYPWTSGRVGNCTGTGPCWDYQYHLNGDIGLSLIYQWVTSGDTDTFREKHFPIYDSVATLYSNLVEPNGTSWTLTNMTDPDEYANHVDAGGFTMPMISETLEYANAFRQQFGFEMNETWSEIADNVLILRENGVTLEYTTMNGTAVVKQADVVLATYPLVYDNYTSQSSLTDLDYYANKQSADGPAMTWAIFSIVAGAVSPSGCSAYTYQQYSFAPYARAPFFQLSEQMIDDASINGGTHPAYPFLTGHGGANQVVLFGYLGLRLLPDEAIHIEPNLPPQIPHITYRIFYWRGWPISARSNYTHTVIQRAAHAPPLDTADHRFANASIPVYVGPESNATVYTLPIRRPLTVQNRQIGTINSIPGNLVQCTPVFSPDDFEPGQFPLSIVDGATSTRWQPKSANPSSVTVQLSAASRHLQTMASGFHFEWAQAPPVNATVIFHDQPLQNPALALTATPPGARIVASLTNIKQSLPYSEQTVDSNQVSLPVGNTTTIQLDVPVPVSRYATLLISGNQALSGAHDDTGATVAEWAILGPGSQVDQTKSTRTMSSRDTATLKRLNRGGGAMINY

SEQ ID NO: 29 - protein, 687 aa, Rhizoctonia solani
LPQAETSTSTSGTSTVTGSTTASEPISTAPATETTAVSTAVPSPTAPLGSPLPRQAALPPKQAWCPSEIFCAGQLLQSVNLAKLYVDSKTFVDKPTAFDAQRVLSDFNALGPQDNVTVGAIANFVSNDFRGEGLELEALTLSNFPENPTFLSKIKDPLVKAWSKIVHTYWSDLIRGTNPETLCSDRNGTTGCESSLIPLNHTFVVPGGRFREQYYWDSYWIVRGLLESQLYDIVNSTLQNFMDELDTIGFIPNGGRIYYLNRSQPPLFIHMLAAYVNRTKDTDILDRALPLAEKELAWWANNRTFKVESPTSKKTYTVYRYAVNNTAPRPESYLPDYITANGEDIETPLTDEQKADLYAELATGAESGWDYTARWSRQQFSGNLSNTEPQLRSLNLRALVPVDLNAILYGAHIQLASLFDRHTKSKRDLRARASASSYRKKADTLKKAILDLCWNEQKLAFYDFNTTAGGQSSTFTAAAFYPLWMGIWPESLLKSETKTFGAFSSVNYVLNMYNGTYPATFLETGLQWDFPNSWPPHVYIILEALNNIPKKLNKQKLPQINSTVTSFDLVPEGQLGLSEDQLPKQTLDLGGYAATDINAGNNTVINGGTPAKNEKWRDSMTRQLANRYVSAAFCSWYSTGGSIPGLLQQLSPEELNATNSDPSSEGHMFEKVYLIHFMFPPLDPHKC

SEQ ID NO: 30 - protein, 530 aa, Achlya hypogyna
ADIEPKDIYCSGPVLETIQEARLFNDSKHFVDMVMKAAPQTVLSAFEALPDHSNTTLKAFLDKYFDEPSTDLVEIELPDFKESPAPLQAIKDADLKAWALQINKLWKLLGRKRVLPDGHYGSHLPTKHNLVVPGGRFRESYYWDSYWIVLGLLKSDMAETAKGVVQNLLDFVDAYGFVPNGGRIYYLNRSQPPLLSDMVRAIFEATKDEAYLAQALPLLDKEYAFWMTQGAATHRVEVQAKDGKTYSLNRYFSAGTSPRPESFREDIETASLVPDASRPTLYQNIIAAAESGWDFSSRWFQDGKTMKSLYTTDVIPVDLNAIMYRFERNLASFHKHVGNAQKAVAMDSAADARRAAIDAVLWNDAAGAWKDYITSAKAHSTIVSISDYTPLWAQAFDATDAARNARILASLKSSGLVLVAGIQTTTAHTGQQWDAYNAWAPEIDFTVEGLLRLNASEATAYAGKIVSDWVATGHSAYKQTGYMLEKYNASVVGGLGSGGEYDLQFGFGWTNGVILKFLTEYQDLLQHDHC

SEQ ID NO: 31 - protein, 753 aa, Schizopora paradoxa
MPQAGSSTATSPGISTGVPSITLSTSVPAPTIPLTNDVPSQAPLPPVQAWCPSKIFCAGSLLQTVNVASLYADPKTFVDKPTNASSQTVLANFNALVASAGNSTSNITEQTMVNFVDSNFRGEGLELEALALPNFTPNPPFLQNITDPLSKAFAQTVHGFWTQLIRGTNSSTLCGEGTNSGSCESTLIPLNHTFVVPGGRFREQYYWDSYWIIQGLVQSQLLDIANATLQNFMDELEQFGFIPNGGRLYYLNRSQPPLFIHMLFDYVQASNDSSILTRALPLAEREFDFWATNRTLNVTSPFTNKTYQVSRYAVNNTAPRPESYLTDYSTANGPDISLNETQKEALYAELASGAETGWDYTVRFASQPFAGGTNNTNPILRTLAIRETIPICLNSILYKAHVLLASLYSEPFSSSTNTTAKERAAFHTGAADQLKSAILDLFWDSNKLAFYDFNTTSMTRNSIFTTAHFYPMWNGIFPDELLSNETAAFGAFSSINMVMNKFNGTFPTTFIESGLQWDAPNAWPPHQFIALQALQNVPMNISTKPVPATPSGQTAFSLIPSGQLGLSEMQLPGQPIKGGSNASATADTNALNGTVVNGGNATGNEPWSVTLQREMANRYFTSALCSWHATGGSIPNVLARLSDAELAITNSQNNTGNMFEKFSYSDVDSSGGGGEYTVQAGFGWTNGVVLWVASTYGNVLNSPQCPPLLVSTGSSSSSGGGGSSGGNSAGHRTAPAPFALALAAAMLVAFIGM

SEQ ID NO: 32 - protein, 515 aa, synthetic construct
  REGION 1..515: note = Saccharoymycopsis fibuligera A40N glucoamylase
  SIGNAL 1..26
MIRLTVFLTAVFAAVASCVPVELDKRNTGHFQAYSGYTVNRSNFTQWIHEQPAVSWYYLLQNIDYPEGQFKSAKPGVVVASPSTSEPDYFYQWTRDTAITFLSLIAEVEDHSFSNTTLAKVVEYYISNTYTLQRVSNPSGNFDSPNHDGLGEPKFNVDDTAYTASWGRPQNDGPALRAYAISRYLNAVAKHNNGKLLLAGQNGIPYSSASDIYWKIIKPDLQHVSTHWSTSGFDLWEENQGTHFFTALVQLKALSYGIPLSKTYNDPGFTSWLEKQKDALNSYINSSGFVNSGKKHIVESPQLSSRGGLDSATYIAALITHDIGDDDTYTPFNVDNSYVLNSLYYLLVDNKNRYKINGNYKAGAAVGRYPEDVYNGVGTSEGNPWQLATAYAGQTFYTLAYNSLKNKKNLVIEKLNYDLYNSFIADLSKIDSSYASKDSLTLTYGSDNYKNVIKSLLQFGDSFLKVLLDHIDDNGQLTEEINRYTGFQAGAVSLTWSSGSLLSANRARNKLIELL

SEQ ID NO: 33 - protein, 475 aa, Streptococcus mutans
MTKQYKNYVNGEWKLSENEIKIYEPASGAELGSVPAMSTEEVDYVYASAKKAQPAWRSLSYIERAAYLHKVADILMRDKEKIGAVLSKEVAKGYKSAVSEVVRTAEIINYAAEEGLRMEGEVLEGGSFEAASKKKIAVVRREPVGLVLAISPFNYPVNLAGSKIAPALIAGNVIAFKPPTQGSISGLLLAEAFAEAGLPAGVFNTITGRGSEIGDYIVEHQAVNFINFTGSTGIGERIGKMAGMRPIMLELGGKDSAIVLEDADLELTAKNIIAGAFGYSGQRCTAVKRVLVMESVADELVEKIREKVLALTIGNPEDDADITPLIDTKSADYVEGLINDANDKGAAALTEIKREGNLICPILFDKVTTDMRLAWEEPFGPVLPIIRVTSVEEAIEISNKSEYGLQASIFTNDFPRAFGIAEQLEVGTVHINNKTQRGTDNFPFLGAKKSGAGIQGVKYSIEAMTTVKSVVFDIK

SEQ ID NO: 34 - protein, 508 aa, synthetic construct
  REGION 1..508: note = Saccharoymycopsis fibuligera A40N, S42A, S72A glucoamylase
  SIGNAL 1..19
MRFPSIFTAVLFAASSALANTGHFQAYSGYTVNRANFTQWIHEQPAVSWYYLLQNIDYPEGQFKAAKPGVVVASPSTSEPDYFYQWTRDTAITFLSLIAEVEDHSFSNTTLAKVVEYYISNTYTLQRVSNPSGNFDSPNHDGLGEPKFNVDDTAYTASWGRPQNDGPALRAYAISRYLNAVAKHNNGKLLLAGQNGIPYSSASDIYWKIIKPDLQHVSTHWSTSGFDLWEENQGTHFFTALVQLKALSYGIPLSKTYNDPGFTSWLEKQKDALNSYINSSGFVNSGKKHIVESPQLSSRGGLDSATYIAALITHDIGDDDTYTPFNVDNSYVLNSLYYLLVDNKNRYKINGNYKAGAAVGRYPEDVYNGVGTSEGNPWQLATAYAGQTFYTLAYNSLKNKKNLVIEKLNYDLYNSFIADLSKIDSSYASKDSLTLTYGSDNYKNVIKSLLQFGDSFLKVLLDHIDDNGQLTEEINRYTGFQAGAVSLTWSSGSLLSANRARNKLIELL

SEQ ID NO: 35 - DNA, 750 bp, synthetic construct
  misc_feature 1..750: note = Modified tsl1 promoter
  source 1..750: mol_type = other DNA
tctttcgatcactaccatgtctgtttaaccgagcaacgcgttcctccggagccgatggtactggctccggagaagggtcgttggtggcatccgagggcgccggtttggcatcatgttcggttcgcgagggtacttgcttggcgcccctgtgtttcacggtgtaaacaaacaagcacaccatcgccagtataaacactatagtcgatccatccatttttacttttgtgcgcgtaggtagccgtgcctcgcctgtgtgtgtgggaatgtctaaatgtgtcccgagttattgttctaaagcgggcaccattgtagtaacttattgcgaaatttctgctcttctcgtctcgctcaaaaatcgcgttcagggtaaaaggggcgaaacagagggccagatagaaatttcgagaaaagcgggtcacccccgcccctgcattttgatatggcgtatttgggattgcttgctcgaaagtgtctaagtccggctggcgggcctggcgccctcgccgaagggagataggaaggggcgggggtccgggcagcggctatggtgtcagttacctagggaaggagaagggggtagaaccaaggggctagcacactcaccctggggccctcgtctagccaagcttaaatataaatactaatgtaactataaatataaggatctaccgtgtcattgcacatccacccacccgtcgattaaaaaaccaaacaaagcaaagaatacaatagcaacgcaagatcaacaca

SEQ ID NO: 36 - DNA, 750 bp, Saccharomyces cerevisiae
  source 1..750: mol_type = genomic DNA
tctttcgatcactaccatgtctgtttaaccgagcaacgcgttcctccggagccgatggtactggctccggagaagggtcgttggtggcgaccgagggcgccggtttggcatcctgtacggttcgcgagggtacttgcttggcgcccctgtgtttcacggtgtaaacaaacaagcacaccatcgtcagtataaacactatagtcgatccatccatttttacttttgtgcgcgtgggtagccgtgcctcgtctgtgtgtgtgggaatgtataaatgtgtcccgagttattgttctaaagcgggcaccattgtagtaacttattgcgaaatttctgctcttctcgtctcgctcaaaaatcgcgttcagggtaaaaggggcgaaacagagggccagatagaaatttcgagaaaagcgggtcacccccgcccctgcattttgatatggcgtatttgggattgcttgctcgaaagtgtctaagtccggctggcgggcctggcgccctcgccgaagggagataggaaggggcgggggtccgggcagcggctatggtgtcagttacctagggaaggagaagggggtagaaccaaggggctagcacactcaccctggggccctcgtctagccaagcttaaatataaatactaatgtaactataaatataaggatctaccgtgtcattgcacatccacccacccgtcgattaaaaaaccaaacaaagcaaagaatacaatagcaacgcaagatcaacaca

SEQ ID NO: 37 - protein, 391 aa, Candida albicans
  SIGNAL 1..23
MFLKNIFIALAIALLVDASPAKRSPGFVTLDFDVIKTPVNATGQEGKVKRQALPVTLNNEHVSYAADITIGSNKQKFNVIVDTGSSDLWVPDASVTCDKPRPGQSADFCKGKGIYTPKSSTTSQNLGTPFYIGYGDGSSSQGTLYKDTVGFGGASITKQVFADITKTSIPQGILGIGYKTNEAAGDYDNVPVTLKNQGVIAKNAYSLYLNSPNAATGQIIFGGVDKAKYSGSLIAVPVTSDRELRITLNSLKAVGKNINGNIDVLLDSGTTITYLQQDVAQDIIDAFQAELKSDGQGHTFYVTDCQTSGTVDFNFDNNVKISVPASEFTAPLSYANGQPYPKCQLLLGISDANILGDNFLRSAYLVYDLDDDKISLAQVKYTSASNIAALT

SEQ ID NO: 38 - protein, 327 aa, Aspergillus niger [sequence omitted]

SEQ ID NO: 39 - DNA, 750 bp, Saccharomyces cerevisiae
  source 1..750: mol_type = genomic DNA
agaaacgaatgtatatgctcatttacactctatatcaccatatggaggataagttgggctgagcttctgatccaatttattctatccattagttgctgatatgtcccaccagccaacacttgatagtatctactcgccattcacttccagcagcgccagtagggttgttgagcttagtaaaaatgtgcgcaccacaagcctacatgactccacgtcacatgaaaccacaccgtggggccttgttacgctaggaataggatatgcgacgaagacgcttctgcttagtaaccacaccacattttcaaggggtcgatctgcttgcttcctttactgtcacgagcggcccataatcgcgctttttttttaaaatgcgcgagacagcaaacaggaagctcgggtttcaaccttcggagtggtcgcagatctggagactggatccttacaatacagtaaggcaagccaccatctgcttcttaggtgcatgcgacggtatccacgtgcagaacaacatagtctgaagaagggggggaggagcatgttcattctctgtagcactaagagcttggtgataatgaccaaaactggagtctcgaaatcatataaatagacaatatattttcacacaatgtgatttgtagtacagttctactctctctcttgcataaataagaaattcatcaagaacttggtttgatatttcaccaacacacacaaaaaacagtacttcactaaatttacacacaaaacaaa

SEQ ID NO: 40 - DNA, 750 bp, Saccharomyces cerevisiae
  source 1..750: mol_type = genomic DNA
ttgcaagaaaaggcataattgtggcgaggtaatgaaaaatttttgacgtttccctcattcattcatacataacattttgggattttggaacaaggcgagaggagaacgtctttggaccactgtaatagtacacaatgcaagatattttaagtttatgctaaaaatccagaagtgacgcttcatcaacggtgtcaatatggaccgaattttagtgtatcttcaattgtaaccgtgaggagtagactctttgaatagggggaagaagaaatatcatattcaaagctaattcattgaaattagtgcttgtctcatctagcctttagtggttaatctctggaggagcacatatggggttaaagccatgccgggactgggggcccctatcggggctcgaacccgaatcccgcgagtatttatttgaaggtccgggacgcaagttacctaatctggttaattgatatcccatttaggcgatgacgttccttcccctcacccctcggcttgttagaagatctattgttatagcctcctctggaagaatttatgccagatgaagaaaaaaacttctcgaagttcccagatgcccaaatgagggctttccatccctgttagctggaaaagtgtaagtatatctatataaaaagtcggcctacttttgccaggttcgtctttcacttgcactctcttgatcttactttctactcaaaaagaatccaatacacaaaaataaaatcagtactattactaata

SEQ ID NO: 41 - DNA, 750 bp, Saccharomyces cerevisiae
  source 1..750: mol_type = genomic DNA
tttttcatttttttacttttaccccgcatcctgcaaaccccggaaattttattaggaataatttatttcccggttggaaataaaccggaaaaatgaagatttagacgcttttaaggagtacgtgttcctgtcgtttatccactaagtatactggttgcccctggccagatctcagtatagcagtgacgcgtgggtttcaggaagaatggcagtccccttttgtttttccgcattgtgtgagcttcttatgtccctgaaccccactattctgcccctttgaaactcccgcacgtgtgccccgtttgttggaagataacgaaataccttactggagcaaccaggaaaaatactctggttgcaaaaaccaacaaaagaaaaaatagaagacctaagaactatgcatttttttttaaagggttgatgaaaaaaaaaaagtttctttctccccccggattttggtaccttaggaccgttgagaggaatagtaacaagtgaacgcaacaaagattgttctcaactgcttctgtttcctccttttctttaaagaggaatatttcatatataagcaatcggtttcacttccttgggaatattctaccgttccttcatcttgtattcttctctttctcttagcgtaatatatagcagaaaagcaataagaaacaattgtggcttgcaatactcaattagaattcttttcttttaatcaaactcacccaaacaactcaattagaatactgaaaaaataag

SEQ ID NO: 42 - DNA, 750 bp, Saccharomyces cerevisiae
  source 1..750: mol_type = genomic DNA
gttgcctggccatccacgctatatatacacgcctggcggatctgctcgaggattgcctacgcgtgggcttgatccaccaaccaacgctcgccaaatgaactggcgctttggtcttctgccatcgtccgtaaaccccggccaaagagaccggaaagatcggtgaaaacatcttgatcttgctcccgggaattttagattcaggtaggaaattgattacatcaatactgttaccctgaatcatattcgacgatgtcgtctcacacggaaatataattcatttcttggttttccaaaaaaattttcattttttttcacttttttgtttcgtcctcctttttttttttttgttttattttttgtcctgtgttcacctttttttttttcagtttacatctttctgcattcttttctgtgtttttttttttttttcgtttttccattgttcgttcgttgcctgttttttcgccctattgttctcgagcctaaaaattttttcctttcctgctttcctttcttcgttcaaagtttcctattccattgttctctttggtaaactcattgttgtcggaactcagatatattcaggtcaatttactgtacttcaattgacttttttcttgaaatttcaacttgccttttcaacttgttcttcttttttaatcttattctacactttagttcccttaccttgttcctaattattgtctagcaaaaagaaaacatacacctatttcattcacacactaaaac

SEQ ID NO: 43 - DNA, 750 bp, Saccharomyces cerevisiae
  source 1..750: mol_type = genomic DNA
ggtaattggagtgcgccggcccgtgctttcagaggaacaaaggaagaattgttaaaaaaaaggcagtgacaacgagcacgggtgcaccaaatggtgtgataggcaacctaggacaaaaagaagtgcgcagttcccgtgtgcggcgccaagacaaatgtttcacatgcctcctcaaggggctacgctacctagcctcaccacccgatctctttttttcctgaacgcagaggggaccgtacgaagaaaaatgttttttaggcaacggagattcgttttatccacgtttaccccacaaaaagtgcaggtacattgtggggccccggcatcgaaaaccagtttttttcctttaaacgctggaaaaaaaggagaaattgttggaactttgcagagaatagtccgtaggcaaattgaaaatgttccttaaaaaatttcatttcttactcattgagtattgagattattcagatgccctccgtgccttcattgaaaaaaatccaagagatgtctcggatttgtatgcagattttggtttgcagacaatggagagcaaatgggtatacaatatagaaagcacagaaacatataaaaagagctcgagaaaagacatatggttcgtaactatcttcttcttttttccaattttttctgttttaataataaaaaaaacaagaacaaacaagctcaacttgtcttttctaagaacaaaaacaaaaacaactaaacaagaattttcctaattttactttaagg

SEQ ID NO: 44 - DNA, 750 bp, Saccharomyces cerevisiae
  source 1..750: mol_type = genomic DNA
gagttatcttattcattggtgacggttctttgcaattgactgttcaagaaatctccaccatgatcagatggggcttgaagccatacttgttcgtcttgaacaacgatggttacaccattgaaaagttgattcacggtccaaaggctcaatacaacgaaattcaaggttgggaccacctatccttgttgccaactttcggtgctaaggactacgaaacccacagagtcgctaccaccggtgaatgggacaagttgacccaagacaagtctttcaacgacaactctaagatcagaatgattgaaatcatgttgccagtcttcgatgctccacaaaacttggttgaacaagctaagttgactgctgctaccaacgctaagcaataagcgatttaatctctaattattagttaaagttttataagcatttttatgtaacgaaaaataaattggttcatattattactgcactgtcacttaccatggaaagaccagacaagaagttgccgacagtctgttgaattggcttaagtctgggtccgctcctttctaaatttgaagaatttctcttaaacgatatgtatattcttttcgttggaaaagatgtcttccaaaaaaaaaaaaccgatgaattagtggaaccaaggaaaaaaaagaggtatccttgattaaggaacactgtttaaacagtgtggtttccaaaaacctgaaactgcattagcgtaatagaagactagacacctcgatacaaata

SEQ ID NO: 45 - DNA, 750 bp, Saccharomyces cerevisiae
  source 1..750: mol_type = genomic DNA
atagattcattattcactgcatcacatacatttttgctttggagttcttaatcaactttctatgtgaagcgtaaaatcaatagttaccttgtattctctgttgatcactgtccaaacctatatttttgtttccccctcagagaagttaggtcgatgcctatcgtagtttgcaaggaagacggcttccaactgagaccgccctagtctggcaggattcgtgaaaggagtccatgccagctgctacccagggattgccacggccccggccagcgtatagtacactgtcaggagcaattcgaagaggcaaccaaaattacgctaagcatcgcactctctctcagtctggtgctaagcggaagacatgcttccaatggcctcctcaccgagaacggatattaaacgaaacgaagaaaaaaaatcttcacggaaaatgcgtaatgtctggatgacaaaatgcatgggtgtaaaaaaggaaatgagacgaacttctattacccttagtgggttgacgaattttgaaataaagtttttccttttttttttttttttctttttcattgtttggttgccttcaaattacatataagatttctcgagaagggttttccattgttcttttcattaggcgttgaagtgcatctaaagtgcgcttgaatgatttcagatagaaagactaaagaagtggtgtgagtataattaactcaattgaagacggtttacctgaagtgatatactgtgccttgagaaaca

SEQ ID NO: 46 - protein, 391 aa, Candida dubliensis
MFLKNIFIALAFALLVDATPAKRSAGFVTLDFEVIKTPVNATGQDGKVKRQAIPVTLNNEVVSYAADITVGSNRQKFNVVVDTGSSDLWIPDASVTCENPPPGQSADFCKGKGLYTPKSSTTSQRLGNPFYIGYGDGSSSHGTLYKDTVGFGGASITKQVFADVTKTSVNQGILGIGYKTNEAAGDYDNVPVTLKKQGVIAKNAYSLYLNSPNAATGQIIFGGVDKAKYSGSLIAVPVTSDRELRITLNSIKAAGKNINGNIDVLLDSGTTITYFQQDVAQGIIDAFHAELKQDGNGNSLYVADCQTSGTVDFNFANNAKISVPASEFTASLFYTNGQPYPQCQLLLGINDANILGDNFLRSAYIVYDLDDNEISLAQVKYTSASNIAALT

SEQ ID NO: 47 - protein, 394 aa, Candida tropicalis
MFLSQLVVFLVFGLLVTASPTTSPPGFISLDFVIIKTQKNIVPNENIIVSKRQPVPVTLIKEQIAYAAEITIGSNNQKQTVIIDTGSSDLWVVDKNATCVRRFEQQVQDFCKANGTYDPITSSSAKKLGTVFDISYGDKTNSSGNWYKDTIKIGGITITNQQFANVKSTSVAQGVMGIGFKTNEASNVTYDNVPITLKKQGIISKSAYSLYLNSSDSTTGEIIFGGVDNAKYTGKLIDLPVTSNRELRIYLNSLTIGVTNISASMDVLLDSGTTFSYLQQDVLQHVVDKFNGQLIHDALGNPLHLVDCDLPGNIDFEFSNSSKISVPSSEFAVKLYTINGELYPKCQLSILTSSANILGNNFLRSAYIVYDLEDKKISLAQVKYTSKSNILPLT

SEQ ID NO: 48 - protein, 580 aa, Clavispora lusitaniae
MKFLSLVTLAAAVSGATVENLRREENKQETIVPLRLDFSVLRGSSPQDMAPGRGAALAKRDGQAELTIQNEQTYYSADLKLGSDHQEVSVLVDTGSSDLWIMASDVECYSSQSQSSSTKRSVGDHFGRRRALSEDDLAHALFQEQSDNTPDASQPLQDKRDTESMAFPDIASILESFTIIETNIPQPSGSSSPDVSGGSGGSGGYGGSNTCTSEGSFNTDSSDTFHMNSSAPDFAIQYADGTSARGFWGTDYVSIDTANVSDVSFAVVNETDSGFGVLGIGLPGLETTYSGTSGSYMYENFPMRLKSSGVIHKNVYSLYLNKADAQSGSVLFGGVDHAKYTGQLTTVPLVNIYSKYYKNPIRLDVALDSISFESTSSNITAYKGNLAALLDSGTTYSYLPTSVFERFINVVNAQSSSIGLYQLSCSYNTDSASVVFNFSGAQIKVPLSDLVMTYRNRCYLTVLEQSSSSSSSSSSSTPEYAVLGDNFLRNAYVVYNLDDYEISLAQAKYTDEEDIEIVSSSVPSAVKAGGYSSTSLSESSDTSEVTTLSSSSLKKSGAPRLAPWKEMGAALMVLLAFALM

SEQ ID NO: 49 - protein, 406 aa, Clavispora lusitaniae
MQLSALVAIATALIAGADAKKFSTKLNKVPIEETLDARSFSGYTKSLANKYIGAFGAAGVGAGSGVQQVAEVPFVANSEHEAPLTNYLNAQYFTEIQLGTPGQTFKVILDTGSSNLWVPSRDCSSLACFLHTKYDHDESSTYKANGSEFSIQYGSGAMEGYISQDVLAIGDLVIPKQDFAEATSEPGLAFAFGKFDGILGLAYDTISVNKIVPPVYNAIAQGLLDAPQFGFYLGDTNKNEENGGVATFGGYDEALFKGDLTWLPVRRKAYWEVSFDGIGLGDEYAELTATGAAIDTGTSLITLPSSLAEIINAKIGATKSWSGQYQVDCATRDNLPDLTLTFAGYNFTLSPYDYTLEVSGSCISAFTPMDFPEPIGDLAIVGDAFLRRYYSVYDLKKDAVGLAPAK

SEQ ID NO: 50 - protein, 405 aa, Saccharomyces cerevisiae
MFSLKALLPLALLLVSANQVAAKVHKAKIYKHELSDEMKEVTFEQHLAHLGQKYLTQFEKANPEVVFSREHPFFTEGGHDVPLTNYLNAQYYTDITLGTPPQNFKVILDTGSSNLWVPSNECGSLACFLHSKYDHEASSSYKANGTEFAIQYGTGSLEGYISQDTLSIGDLTIPKQDFAEATSEPGLTFAFGKFDGILGLGYDTISVDKVVPPFYNAIQQDLLDEKRFAFYLGDTSKDTENGGEATFGGIDESKFKGDITWLPVRRKAYWEVKFEGIGLGDEYAELESHGAAIDTGTSLITLPSGLAEMINAEIGAKKGWTGQYTLDCNTRDNLPDLIFNFNGYNFTIGPYDYTLEVSGSCISAITPMDFPEPVGPLAIVGDAFLRKYYSIYDLGNNAVGLAKAI

SEQ ID NO: 51 - protein, 396 aa, Yarrowia lipolytica
MKFTAAVSVLAAAGSVSAAVSKVSINKMSTAELLGKENGFEDHLRMMGQKYMGKFQKLGEFNELASIQDVSNSPLTNYLNAQYYTEIEIGTPPQKFNVILDTGSSNLWVPSVQCNSIACYLHQKYDSAASSSYKANGTAFEIQYGSGSMEGFVSQDTLKLGSLVLPEQDFAEATSEPGLAFAFGKFDGILGLAYDTISVNKIVPPVYNAVNRGLLDKNQFSFFLGDTNKGTDGGVATFGGVDEDYFEGKITWLPVRRKAYWEVEFNSITLGDQTAELVNTGAAIDTGTSLLALPSGLAEVLNSEIGATKGWSGQYTVECDKVDSLPDLTFNFAGYNFTIGPRDYTLELSGSCVSAFTGFDIPAPVGPIAIIGDAFLRRYYSVYDLDHDAVGLAKAK

SEQ ID NO: 52 - protein, 408 aa, Meyerozyma guilliermondii
MKLSISVLGAVAFALFGCADAAVHSAKLNKIPVEETLAAHRFKEYTSGLAAKYLTAFSTSEGITDQTQQQILQQVPFVDGKYDSDLSNYVNAQYFTEIQLGTPGQTFKVILDTGSSNLWVPSADCSSLACFLHTKYDHDSSSTYKANGSEFSIQYGSGAMEGYVSRDTLALGDLIIPRQDFAEATSEPGLAFAFGKFDGILGLAYNTISVNKIVPPIYNAIDQGLLDEPVFAFRLGDTSKDENDGGVATFGGYDKSQFTGKITWLPVRRKAYWEVSFEGIGLGDEYAELTSTGAAIDTGTSLITLPSSLAEIMNTKIGATKSWSGQYQIDCEKRDSLPDLTLNFSGYNFTLSPYDYTLEVGGSCISVFTPMDFPEPIGDLAIVGDAFLRRYYSIYDLKKDAVGLAKSV

SEQ ID NO: 53 - protein, 395 aa, Aspergillus fumigatus
MVVFSKVTAVVVGLSTIVSAVPVVQPRKGFTINQVARPVTNKKTVNLPAVYANALTKYGGTVPDSVKAAASSGSAVTTPEQYDSEYLTPVKVGGTTLNLDFDTGSADLWVFSSELSASQSSGHAIYKPSANAQKLNGYTWKIQYGDGSSASGDVYKDTVTVGGVTAQSQAVEAASHISSQFVQDKDNDGLLGLAFSSINTVSPRPQTTFFDTVKSQLDSPLFAVTLKYHAPGTYDFGYIDNSKFQGELTYTDVDSSQGFWMFTADGYGVGNGAPNSNSISGIADTGTTLLLLDDSVVADYYRQVSGAKNSNQYGGYVFPCSTKLPSFTTVIGGYNAVVPGEYINYAPVTDGSSTCYGGIQSNSGLGFSIFGDIFLKSQYVVFDSQGPRLGFAPQA

SEQ ID NO: 54 - protein, 390 aa, Saccharomycopsis fibuligera
MLFSKSLLLSVLASLSFAAPVEKREKTLTLDFDVKRISSKAKNVTVASSPGFRRNLRAASDAGVTISLENEYSFYLTTIEIGTPGQKLQVDVDTGSSDLWVPGQGTSSLYGTYDHTKSTSYKKDRSGFSISYGDGSSARGDWAQETVSIGGASITGLEFGDATSQDVGQGLLGIGLKGNEASAQSSNSFTYDNLPLKLKDQGLIDKAAYSLYLNSEDATSGSILFGGSDSSKYSGSLATLDLVNIDDEGDSTSGAVAFFVELEGIEAGSSSITKTTYPALLDSGTTLIYAPSSIASSIGREYGTYSYSYGGYVTSCDATGPDFKFSFNGKTITVPFSNLLFQNSEGDSECLVGVLSSGSNYYILGDAFLRSAYVYYDIDNSQVGIAQAKY

SEQ ID NO: 55 - protein, 521 aa, Bacillus subtilis
MGLGKKLSVAVAASFMSLTISLPGVQAAENPQLKENLTNFVPKHSLVQSELPSVSDKAIKQYLKQNGKVFKGNPSERLKLIDQTTDDLGYKHFRYVPVVNGVPVKDSQVIIHVDKSNNVYAINGELNNDVSAKTANSKKLSANQALDHAYKAIGKSPEAVSNGTVANKNKAELKAAATKDGKYRLAYDVTIRYIEPEPANWEVTVDAETGKILKKQNKVEHAATTGTGTTLKGKTVSLNISSESGKYVLRDLSKPTGTQIITYDLQNREYNLPGTLVSSTTNQFTTSSQRAAVDAHYNLGKVYDYFYQKFNRNSYDNKGGKIVSSVHYGSRYNNAAWIGDQMIYGDGDGSFFSPLSGSMDVTAHEMTHGVTQETANLNYENQPGALNESFSDVFGYFNDTEDWDIGEDITVSQPALRSLSNPTKYGQPDNFKNYKNLPNTDAGDYGGVHTNSGIPNKAAYNTITKIGVNKAEQIYYRALTVYLTPSSTFKDAKAALIQSARDLYGSQDAASVEAAWNAVGL

SEQ ID NO: 56 - protein, 351 aa, Ananas comosus
MASKVQLVFLFLFLCAMWASPSAASRDEPNDPMMKRFEEWMAEYGRVYKDDDEKMRRFQIFKNNVKHIETFNSRNENSYTLGINQFTDMTKSEFVAQYTGVSLPLNIEREPVVSFDDVNISAVPQSIDWRDYGAVNEVKNQNPCGSCWSFAAIATVEGIYKIKTGYLVSLSEQEVLDCAVSYGCKGGWVNKAYDFIISNNGVTTEENYPYLAYQGTCNANSFPNSAYITGYSYVRRNDERSMMYAVSNQPIAALIDASENFQYYNGGVFSGPCGTSLNHAITIIGYGQDSSGTKYWIVRNSWGSSWGEGGYVRMARGVSSSSGVCGIAMAPLFPTLQSGANAEVIKMVSET